(19) United States
(12) Patent Application Publication
(10) Pub. No.: US 2002/0132333 A1
(43) Pub. Date: Sep. 19, 2002

(54) Structural analysis of the calpains as procedures for the development of inhibitors

(76) Inventors: Stefan Strobl, Planegg (DE); Carlos Fernandez-Catalan, Planegg (DE); Wolfram Bode, Gauting (DE); Robert Huber, Germering (DE); Koichi Suzuki, Tokyo (JP)

Correspondence Address: Kenneth H. Sonnenfeld, Esq., MORGAN & FINNEGAN, L.L.P., 345 Park Avenue, New York, NY, 10154, US

(21) Appl. No.: 09/736,860

(22) Filed: Dec. 14, 2000

Related U.S. Application Data
(60) Provisional application No. 60/170,651, filed on Dec. 14, 1999.

(51) Int. Cl. C12N 9/64

(52) U.S. Cl. 435/226

(57) ABSTRACT

The invention provides spatial structures and crystal forms of polypeptides comprising at least one subdomain of a protein from the family of proteins that includes calcium-activated cystein proteinases (calpains). Also provided are methods of preparing these crystal forms, and methods of modeling calpains using the coordinates derived from the disclosed crystal forms. The invention further provides compounds that act as ligands for calpains, methods for identifying such ligands, and methods for using such ligands as inhibitors or activators of calpain activity.

### RELATED APPLICATION

This application claims priority to U.S. Provisional Patent Application Serial No. 60/170,651 filed Dec. 14, 1999, the contents of which are herein incorporated by reference.

The present invention relates to spatial structures and the crystal form of at least one polypeptide per asymmetric unit, at least one polypeptide in the asymmetric unit having at least one (sub)domain of a protein from the family consisting of the neutral Ca-activated cysteine proteinases (calpains), which (sub)domain participates in the catalysis. The present invention furthermore relates to compounds, in particular ligands, having the property of acting as a substrate, pseudosubstrate, activator or inhibitor of a neutral Ca-activated cysteine proteinase (calpain), and methods for identifying such a compound or such a ligand for a neutral Ca-activated cysteine proteinase. In the context of the present invention, the use of such ligands or compounds which act as inhibitors and/or activators of the catalytic activity of a neutral Ca-activated cysteine proteinase as an active substance in drugs or for the preparation of a drug is also disclosed. Finally, the present invention also relates to processes for the preparation of a crystal form comprising at least one polypeptide which has at least one (sub)domain of a protein from the family consisting of the neutral Ca-activated cysteine proteinases, which (sub)domain participates in the catalysis. The present invention furthermore relates to methods which permit the modeling of calpains of unknown structure using structural coordinates of a spatial structure or crystal form according to the invention.

The so-called calpains belong to a family of intracellular, Ca-dependent cysteine proteinases which comprise both a plurality of tissue-specific isoforms (n-calpains) and two ubiquitous isozymes (μ- and m-calpain). Calpain belongs in the enzyme class EC 3.4.22.17, it being an enzyme which is present as a heterodimer composed of a large catalytic and a small regulatory subunit (Ono et al., Biochem. Biophys. Res. Com. 245, 289-294, 1998). Appropriate investigations have shown that the large subunit has a molecular weight of about 80,000 and the small subunit one of about 30,000 Dalton.

Ohno et al. (Nature 312, 566-570, 1984) have described the primary molecular structure of chicken μ-/m-calpain by cDNA cloning. The large catalytic subunit of the calpain heterodimer was subdivided into domains with the designations I, II, III and IV, and the small regulatory subunit into two domains with the designations V and VI. Each of the two subunits has a calmodulin-like Ca²⁺ binding domain, which is to be found at the C terminus in each case (domains IV and VI). The domain II of the large subunit, which in turn breaks down into two (sub)domains (IIa and IIb) displays sequence similarities to catalytically active domains of other cysteine proteinases, such as, for example, papain and cathepsins. Apart from three amino acid residues in the active center of the catalytic (sub)domains of calpains, there is nevertheless no pronounced sequence homology with other cysteine proteinases, which is why the calpains are regarded as an independent family, separated through evolution, within the large family of the cysteine proteinases (Berti and Stora, J. Mol. Biol. 246, 273-283, 1995). A regulatory effect on the catalytic activity of calpain was attributed to the calmodulin-type calcium binding domains at the respective C termini of the catalytic or the regulatory subunit (Suzuki et al., Biol. Chem. Hoppe-Seyler, 376, 523-529, 1995).

The proteolytic function of calpains is of the greatest importance for cytophysiology. For example, a key role in the regulation of cellular functions was attributed to the ubiquitously and constitutively expressed μ- and m-calpains. On the other hand, tissue-specific calpain homologs (for example n-calpains) appear to be of key importance for the respective tissue development and function and the existence of tissue (Sorimachi et al., J. Biol. Chem. 264, 20106-20111, 1989). Thus, there are, for example, indications that muscle-specific calpain is involved in the genesis of muscular dystrophy (Richard et al., Cell, 81, 27-40, 1995). Furthermore, the formation of plaques in Alzheimer's patients appears to be due to a deregulation of μ-calpain and its physiological inhibitor calpastatin (Saito et al., PNAS USA, 90, 2628-2632, 1993). The abnormal processing of transmembrane amyloid precursor protein, which is characteristic of Alzheimer's disease and finally leads to the self-aggregation of β-amyloid peptides, is thus attributed to extra- and intracellular proteolytic activities, a possible cause being a loss of balance between intact and autolyzed μ-calpain. Since calpain is evidently also involved in cataract formation (David et al., J. Biochem. 268, 1937-1940, 1993), results of three-dimensional structure elucidations, for example on the basis of corresponding spatial or crystal forms, should provide closer insights into the functioning and the type of regulation of calpains.

Various experiments on overexpression, crystallization and/or X-ray structure analysis indicate the interest in this respect in the elucidation of the structure/function relationship for calpains. Thus, for example, Blanchard et al. (Nature Structural Biology, Vol. 4, No. 7, 532-538, 1997) have cloned, expressed, purified and finally crystallized the calcium-binding domain of the small subunit of rat calpain (domain VI) (Graham-Siegenthaler et al., J. Biochem., 269, 30457-30460, 1994). This isolated domain VI is present in solution as a homodimer and was crystallized in space group C222₁. The crystals have two monomers per asymmetric unit. Although the structure elucidation of domain VI clearly revealed that a folding pattern of five EF hands, a characteristic supersecondary structural pattern having α-helices, is present per monomer, three of which in turn bind calcium in physiological calcium concentrations, the structure described by Blanchard et al. does not enable a functional or structural relationship to be demonstrated between the calcium binding and its effect on the catalytic (sub)domains in the large subunit of calpain. The structural coordinates of the crystal structure solved by Blanchard et al. have been deposited in the PDB database (Brookhaven, USA), under the designations 1AJ5 and 1DVI.

Lin et al. (Nature Structural Biology, Vol. 4, No. 7, 539-547, 1997) likewise describe the crystallization of domain VI, i.e. of the calcium-binding domain of the small subunit of porcine calpain, at a structural resolution of 1.9 Å. This investigation, too, is thus limited to the elucidation of the structure of the calcium-binding domain and provides no information about the structure of the catalytic subunit or its effect on the regulatory mechanism of the catalytic subunit itself. Lin et al. have deposited their crystal structures under the designation 1ALV and 1ALW in the PDB database (Brookhaven, USA).

Finally, it was shown that m-calpain from the rat can be crystallized in two crystal forms, P1 and P2₁ (Hosfield et al., Acta Crystallographica Section D, Biological Crystallography, D55, 1484-1486, 1999). The recombinant rat m-calpain used differs slightly from the natural enzyme. The natural amino acid residue Cys105 residing in the active center was mutated to a serine in the recombinant protein, with the result that the activity of the enzyme was switched off and hence autodegradation was avoided. Moreover, the large subunit was provided with 14 amino acid residues at the C terminus, including a histidine tag. Although Hosfield et al. report X-ray crystallographic data collection with resolutions of up to 2.6 and 2.15 Å, respectively, for the crystals obtained, the authors disclose no crystal forms, i.e. no structural results of the X-ray crystallographic data collection. In addition, the investigations by Hosfield et al. were carried out exclusively for m-calpain from the rat but not for human m-calpain.

Although Masumoto et al. (J. Biochem. 124, 957-961, 1998) describe overproduction of recombinant human calpain in the active form in a baculovirus expression system and its purification and characterization, the authors cannot report either on the crystallization or on an elucidation of the structure of the overexpressed human m-calpain.

The object of the present invention is therefore to provide spatial and preferably crystal forms of calpains, which permit a structure/function investigation. A 3D structure elucidation of a polypeptide or of a complex which comprises at least one (sub)domain participating in the catalysis of the natural calpain, possibly also further catalytic and/or regulatory (sub)domains of one or both subunits, is required for this purpose. It is a further object of the present invention to provide those compounds which can act as substrates, pseudosubstrates, activators or inhibitors of a neutral Ca-activated cysteine proteinase. Moreover, it is the object of the present invention to provide methods for identifying a compound which can act as an agonist or antagonist or substrate, pseudosubstrate, activator or inhibitor for one or more calpains. It is additionally the object of the present invention to provide processes for the preparation of a crystal form with at least one polypeptide which has at least one (sub)domain of a protease from the calpain family, which (sub)domain participates in the catalysis. In addition to such crystallization processes, it is also the object of the present invention to provide crystals which comprise the above-mentioned polypeptides in symmetrical arrangement. Finally, it is the object of the present invention to provide those methods which serve for determining three-dimensional structures of previously structurally unsolved proteins (polypeptides) or complexes having a structural relationship with calpains of known spatial or crystal form, and to provide processes which make it possible to provide agonists or antagonists in the form of pseudosubstrates, substrates, activators or inhibitors for three-dimensional protein structures modeled in this manner.

The above-mentioned objects are achieved by claims 1, 25, 35, 41, 43, 44, 45, 49 and 50.

According to claim 1, spatial forms which represent a three-dimensional structure of at least one polypeptide per asymmetric unit are provided, at least one of these polypeptides per asymmetric unit having at least one (sub)domain participating in the catalysis of the proteolytic calpain reaction of a protein from the family consisting of the neutral Ca-activated cysteine proteinases (calpains). Here, a spatial form is understood as meaning the three-dimensional structure of a molecule or of a molecular complex, i.e. the spatial atomic arrangement of the atoms of the molecule, the three-dimensional appearance of a molecule in the present case, i.e. of at least one polypeptide of the above-mentioned type, as obtained after a structure analysis by the relevant methods for structure elucidation. The methods of X-ray structure analysis of crystals and of the structure elucidation by nuclear magnetic resonance spectroscopy (NMR spectroscopy) may be mentioned in particular here. The spatial form thus corresponds to the three-dimensional appearance of the molecule/molecular complex investigated, i.e. its spatial form represented by the structural coordinates of each atom of the molecule/molecular complex. In a preferred case according to the invention, namely when at least one polypeptide of the above-mentioned type is present in a crystal, the spatial form will correspond to the crystal form of the at least one polypeptide. The crystal form is to this extent also the specific appearance of the crystallized at least one polypeptide in a crystal, as obtained as the result of an X-ray structure analysis on corresponding crystals. Structural coordinates for the atoms of the at least one polypeptide having at least one (sub)domain participating in the catalysis of a calpain reaction reproduce the spatial form of such a molecule/molecular complex whose structure has been elucidated by NMR spectroscopy or X-ray crystallography.

While a spatial form, according to the invention, of at least one such polypeptide can be elucidated by means of NMR structure analysis in solution, it is essential for the X-ray structure analysis method that the molecules to be investigated are present in crystalline form. Such a crystal is characterized by unit cells which present in a characteristic arrangement the molecules/molecular complexes to be investigated. Owing to the laws of symmetry, there are a limited number of such arrangements, which are referred to as space groups.

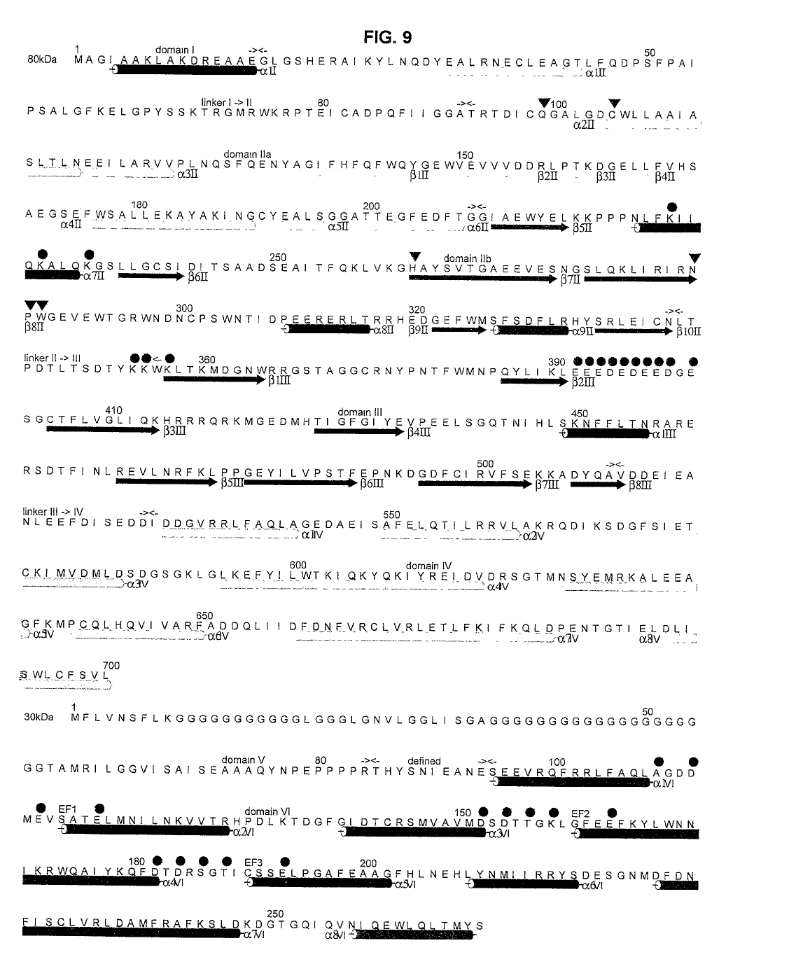

According to the invention, it is envisaged that the preferably crystallized polypeptide contains at least one (sub)domain of a protein from the family consisting of the neutral Ca-activated cysteine proteinases, which (sub)domain participates in the catalysis. According to the standard nomenclature (essentially according to Sorimachi et al., Biochem. J., 1997, 721-732, with slight modifications by the inventor, cf. FIG. 9 and associated description), these catalytic calpain (sub)domains are the two (sub)domains IIa and IIb, from the large subunit of the calpain physiologically composed of two subunits. According to the invention, the spatial form or preferably the crystal form can accordingly represent a polypeptide which can have exclusively an amino acid sequence corresponding to (sub)domain IIa or have exclusively an amino acid sequence corresponding to the (sub)domain IIb or can contain one or both of the above-mentioned (sub)domains in combination with any desired amino acid sequences at the respective termini of the catalytic (sub)domains, i.e. as recombinant protein or for example with domains of other proteins. A spatial form according to the invention, in particular as a crystal form, of a polypeptide which contains the domains I, IIa, IIb, III and IV is in this case preferred. The spatial form, in particular as a crystal form, of a complex of both calpain subunits, i.e. both the 30 kDa and the 80 kDa subunit, is very particularly preferred.

Although in the present case such spatial forms or preferably crystal forms of polypeptides which contain at least one catalytic (sub)domain of a calpain having its natural amino acid sequence is preferred, according to the invention those spatial or preferably crystal forms which are based on nonnatural calpain amino acid sequences, i.e. represent derivatives of the natural sequences, are also disclosed. These are in particular derivatives of the catalytic (sub)domains IIa and/or IIb which have in particular conservative substitution(s) compared with the natural sequences. Conservative substitutions are designated as substitutions in which at least one amino acid has been replaced by another amino acid from the same class. Thus, for example, threonine can be substituted by serine, lysine by arginine (positively charged amino acids), leucine by isoleucine, alanine or valine (aliphatic amino acids), or vice versa in each case. The naturally occurring 20 amino acids are classified according to their chemical or physical properties. Thus, for example, amino acids having positively or negatively charged side chains, aromatic side chains, aliphatic side chains, side chains with hydroxyl or amino groups are grouped in corresponding, respective classes.

A polypeptide in a crystal form according to the invention can, however, also have amino acids which do not occur naturally or at any rate do not typically occur naturally.

However, spatial or preferably crystal forms of polypeptides having at least one deletion and/or one insertion compared with the respective amino acid sequence of one or both catalytic (sub)domains or of a regulatory domain of a calpain are also provided within the scope of the present invention. In particular, the present invention includes derivatives which have at least one insertion and/or deletion in so-called loop structures of the catalytic (sub)domain(s).

The claimed spatial forms, preferably as crystal forms, are preferably three-dimensional structures of polypeptides, the catalytic (sub)domain(s) contained therein originating from isozymes from the family consisting of the ubiquitously expressed calpains or from isozymes of the family consisting of the calpains expressed in a tissue-specific manner (n-calpains). Particularly preferred in turn are spatial/crystal forms for polypeptides or complexes of two or more polypeptides which contain the two subunits with a selection of, but very particularly preferably all, catalytic and regulatory domains. Such three-dimensional spatial forms, in particular crystal forms, are very particularly preferred, as a result of crystallographic investigations, if they contain at least one catalytic (sub)domain of proteins from the group consisting of the m- or μ-calpains. In a very particularly preferred embodiment, the amino acid sequence of the at least one catalytic calpain (sub)domain present in the spatial or crystal form corresponds to a corresponding natural amino acid sequence of eucaryotic cells, in particular of cells of vertebrates, especially of mammalian cells, and here in turn in particular human cells, or derivatives, for example substitution, deletion and/or insertion derivatives thereof.

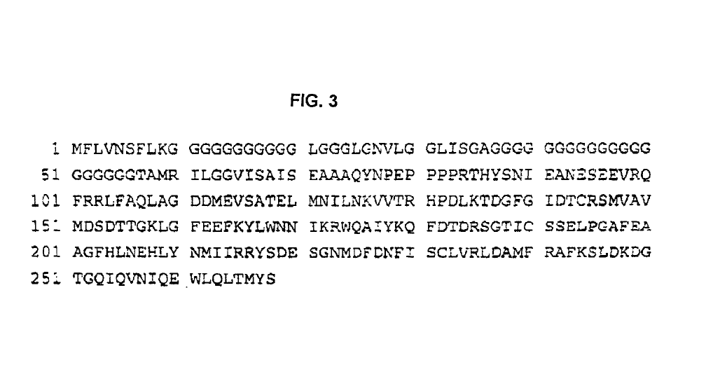

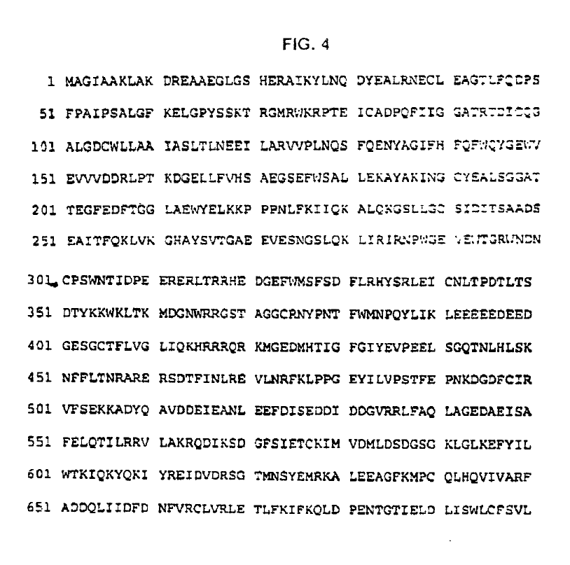

According to the invention, a spatial or crystal form of such a polypeptide which contains the amino acid sequence according to FIG. 3, 4, 5 X and/or FIG. 6, in particular the subdomains IIa (from T93 to G209) and/or IIb (from G210 to N342) contained in FIGS. 4 and 6, or derivatives of one or both of the above-mentioned (sub)domain amino acid sequences, is preferably provided. In addition, a preferred spatial or crystal form will reflect a three-dimensional structure of a polypeptide or of a complex with at least one polypeptide which has an amino acid sequence according to FIG. 9 or a derivative thereof.

In a further preferred embodiment, the present invention discloses those spatial forms, preferably as crystal forms, which, in addition to the at least one polypeptide having one of the amino acid sequences described above according to the invention, has at least one further component. This further component may be one or more identical or different metal ion(s), preferably alkali metal and/or alkaline earth metal ions, especially calcium ions. In the preferred case of a crystal form, the additional component may however also be one or more identical or different heavy metal ion(s), which are typically located in the spatial vicinity of cysteine or histidine residues of the three-dimensionally folded at least one polypeptide sequence. Very particularly preferred here are those crystal forms in which the heavy metal ions, preferably gold and/or mercury ions, interact with one or more of the following amino acids (based on the nomenclature according to FIG. 9, for human m-calpain) C105, C98, H169, C191, R420, C240, H334, C696 and/or H908 (from the small subunit) or with the amino acids of other calpains which correspond structurally and functionally to the above-mentioned amino acids. Also very particularly preferred are crystal forms in which at least one gold and/or at least one mercury ion is (are) complexed by the amino acid C105, by addition to the spatially neighboring amino acids C98/H169, C191/R420, C240/H334 and/or C696/H908 (for nomenclature, see above).

In a further preferred embodiment, the spatial form or preferably crystal form comprises at least one ligand which is (are) typically noncovalently, but optionally also covalently, bonded to the polypeptide(s) in the asymmetric unit. These ligands may be agonists or antagonists of calpain, i.e. substrate, pseudosubstrate, activator or inhibitor molecules. A spatial form, preferably in crystal form, is particularly preferred when the ligand binds to the catalytic domain, in particular at the active center of its calpain domain, or to or at the cleft—in the case of the catalytically inactive conformation—of the at least one polypeptide, which cleft is formed by the subdomains IIa and IIb. A spatial form according to the invention can, however, also have two or more ligands, for example an inhibitory ligand binding to a regulatory domain (domains III and/or IV) and at least one further ligand which docks with one or both catalytic subdomain(s) of the large subunit (IIa and/or IIb).

These functional ligands may be any desired molecules, in particular also organic chemical molecules which can bind to the calpain complex because of their steric and/or chemical properties. Preferred however are di- and/or oligopeptides which are optionally stabilized by chemical modifications, or the di- or oligopeptide analogs, which, for example in the region of the active center, compete for binding sites with the actual (poly)peptide substrate molecules, but which, owing to chemical change, are not subject to proteolysis and hence block the active center of the calpain. Thus, for example, the amide bonds of a di- and/or oligopeptide usually having substrate properties can be modified by reduced amide bonds or pseudopeptide bonds, for example methylene or acetylene groups and are thus not accessible to proteolysis. In a list which is by no means exhaustive, inhibitors of the active center may therefore be nonproteolyzable peptidomimetics of the following peptides (in the one-letter code) or may contain such nonproteolyzable peptide sequences: YCTGVSAQVQK, RARELGLGRHE, AERELRRGQIL, PRDETDSKTAS, KYLATASTMDH, DHARHGPLPRH, STSRTP, SCPIKE, DTPLPV, STPDSP, PNGIPK, PPGGDRGAPKR, WFRGLNRIQTQ and/or RGSGKDSHHPA.

In addition to the spatial forms according to the invention, in particular the three-dimensional crystal forms, which provide a representation of the respective structural constitution of at least one polypeptide having the above-mentioned properties, macroscopic crystals also form a subject of the present invention. Accordingly, crystalline arrangements which serve as a basis for the X-ray structure analysis and have at least one polypeptide per asymmetric unit are disclosed according to the invention, at least one such polypeptide containing at least one catalytic subdomain of a calpain, i.e. either the subdomain IIa and/or the subdomain IIb. Crystals which contain in the asymmetric unit at least one polypeptide which has at least one catalytic subdomain of human calpain, in particular human m-calpain, are preferred. In this context, it is pointed out that those embodiments of crystals according to the invention which are preferred within the scope of the present invention include all those macroscopic arrangements which correspond microscopically in the asymmetric unit to crystal forms as have been disclosed beforehand according to the invention. The preceding disclosure is therefore incorporated by reference to this extent.

Crystals according to the invention may occur in all 65 possible enantiomorphic space groups with respect to their symmetrical properties. According to the invention, particularly suitable space groups are those of the triclinic, monoclinic, orthorhombic, tetragonal, trigonal/rhombohedral, hexagonal and cubic types.

Very particularly preferred crystals are those which contain both calpain subunits, namely the 30 kDa and the 80 kDa subunit, in the asymmetric unit. For example, at least one polypeptide in the asymmetric unit with the amino acid sequence shown in FIG. 4 may be present in a crystal form according to the invention or may contain said crystal form. Very particularly preferred are monoclinic space groups, in particular the space group P2₁. Also preferred are crystals according to the invention having unit cells whose asymmetric unit comprises crystal forms having at least one polypeptide heterodimer, one polypeptide (1) preferably comprising an amino acid sequence corresponding to FIG. 3 and the other polypeptide (2) preferably comprising an amino acid sequence according to FIG. 4. To this extent, the heterodimer preferably present in the asymmetric unit microscopically as a crystal form corresponds to the functional physiological calpain-protein complex with the large and the small subunit. According to the invention, crystals which have a monoclinic unit cell with the following cell constants (approximate dimensions) a=64.9 Å, b=134.0 Å, c=78.0 Å and β=102.40° or a=51.6 Å, b=171.4 Å, c=64.7 Å and β=94.80° are furthermore preferred.

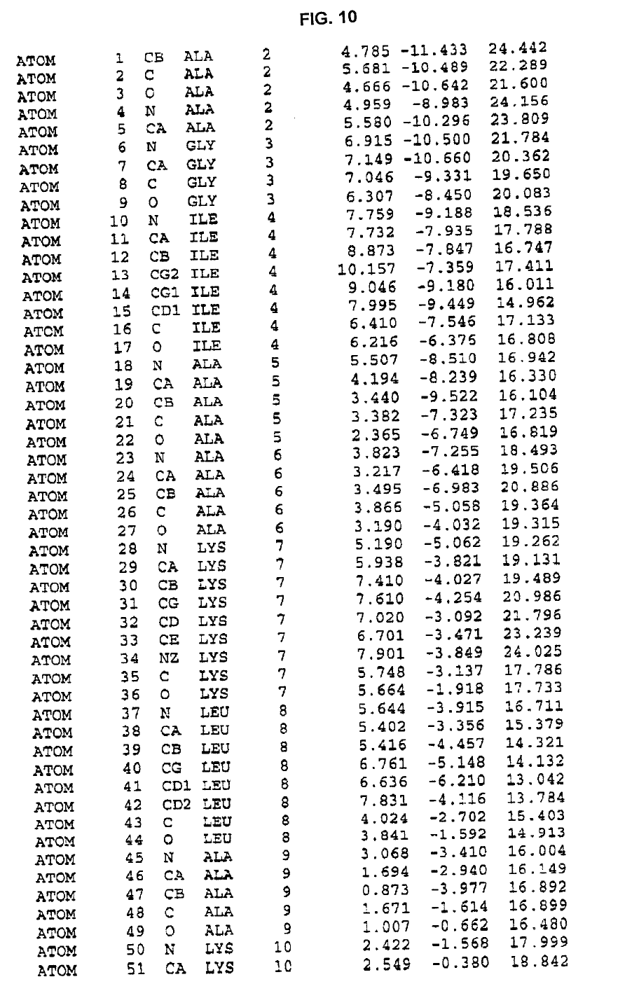

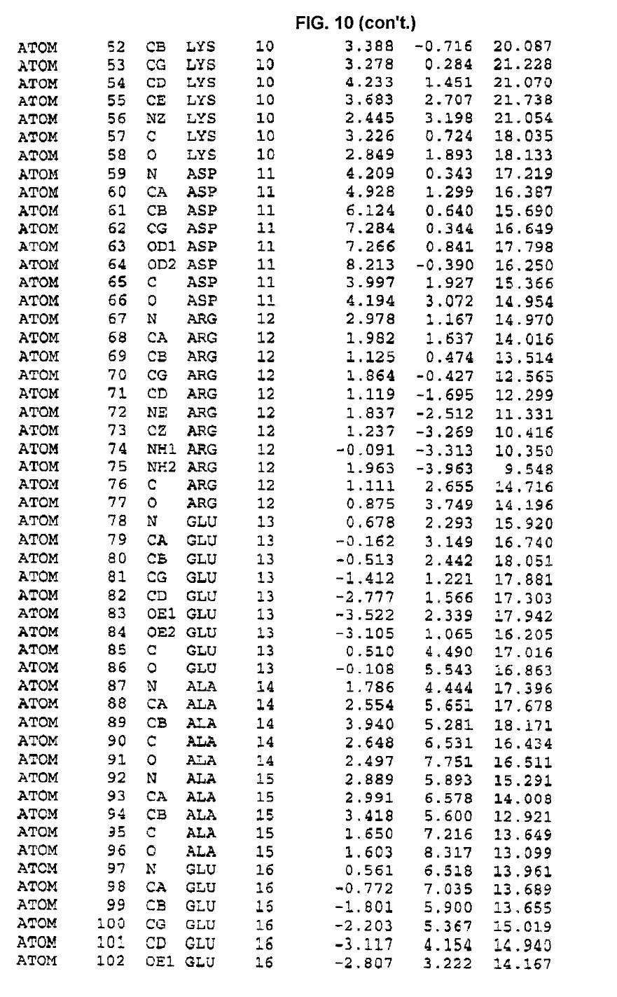

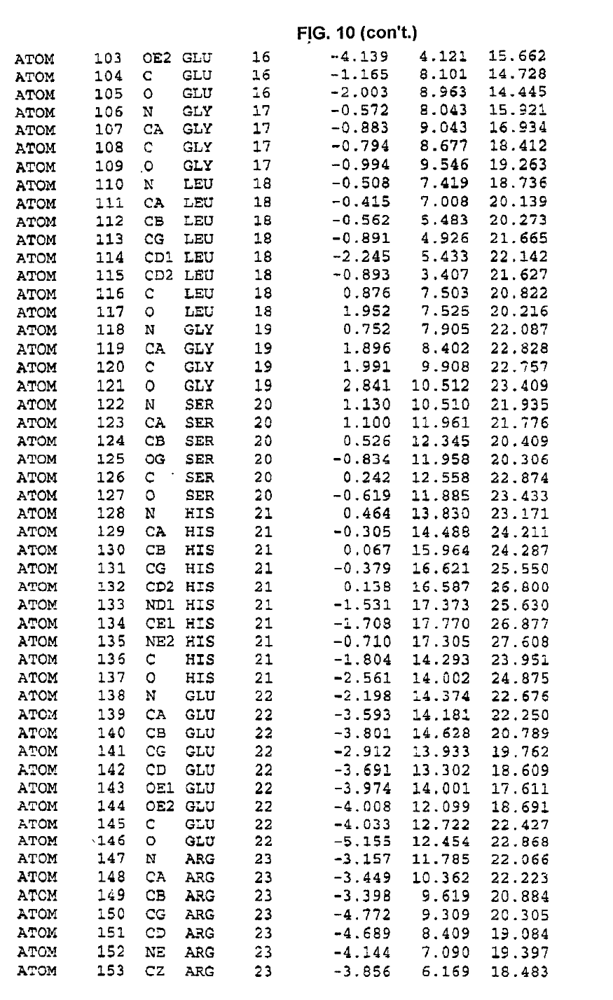

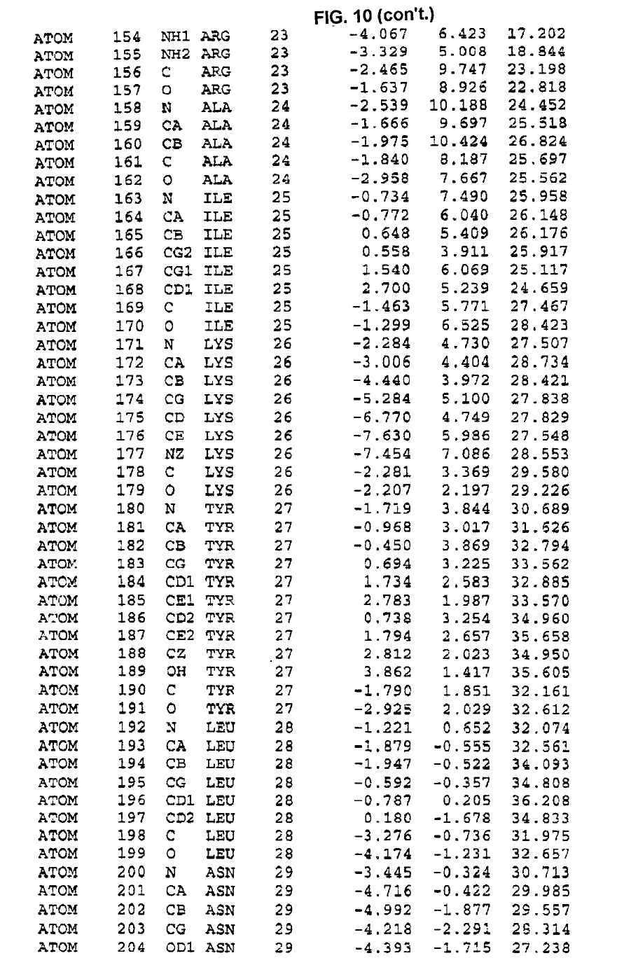

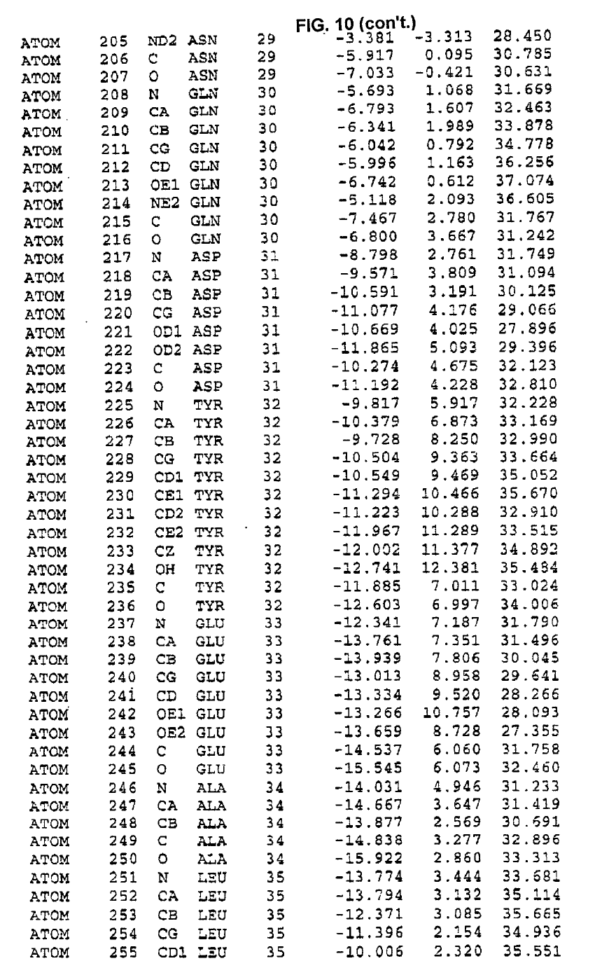

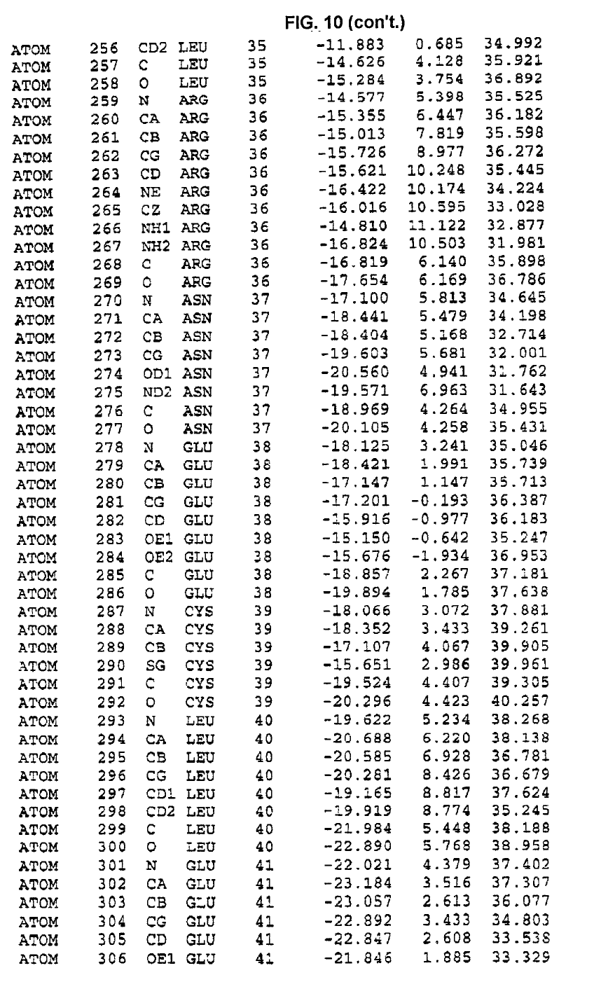

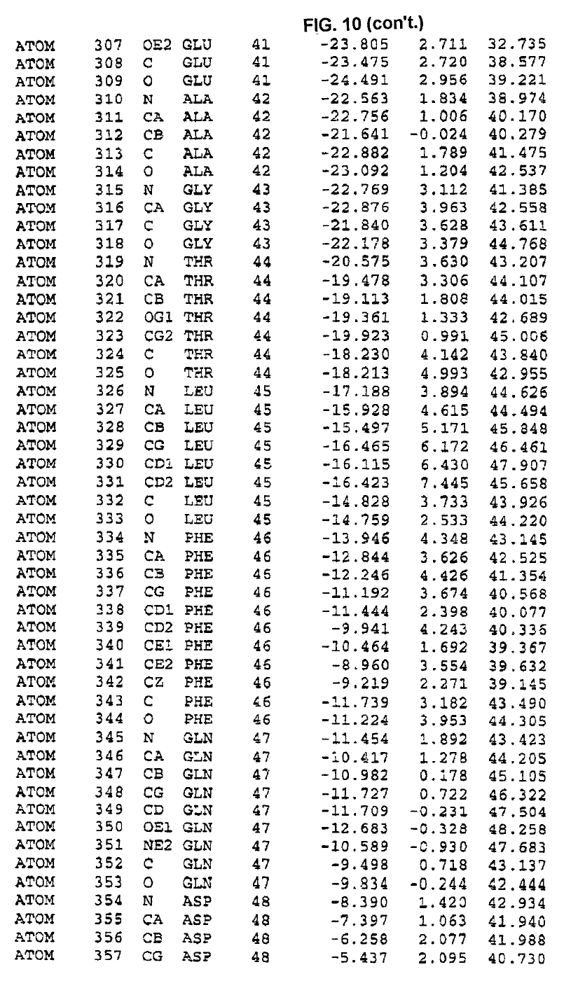

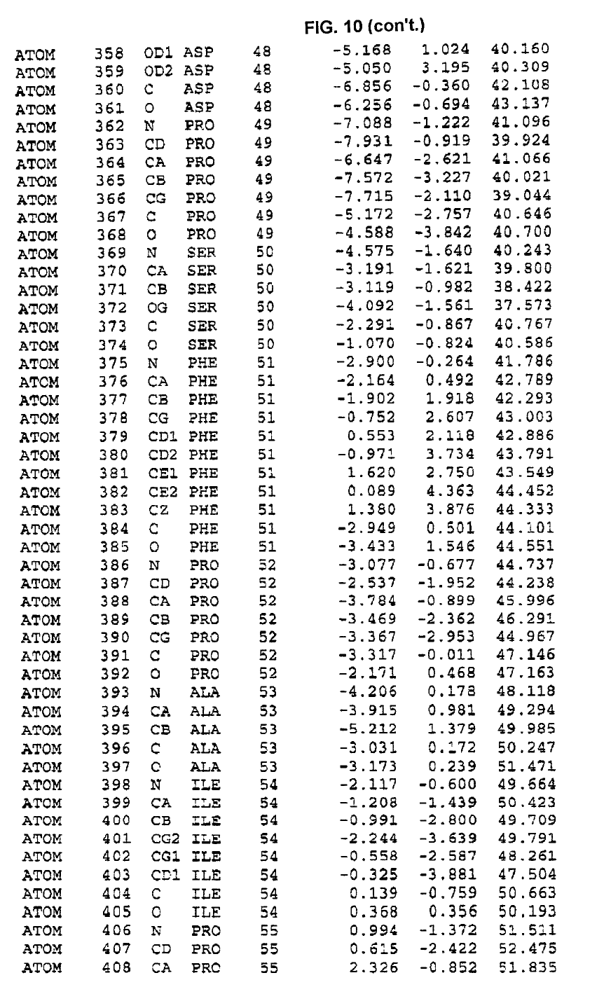

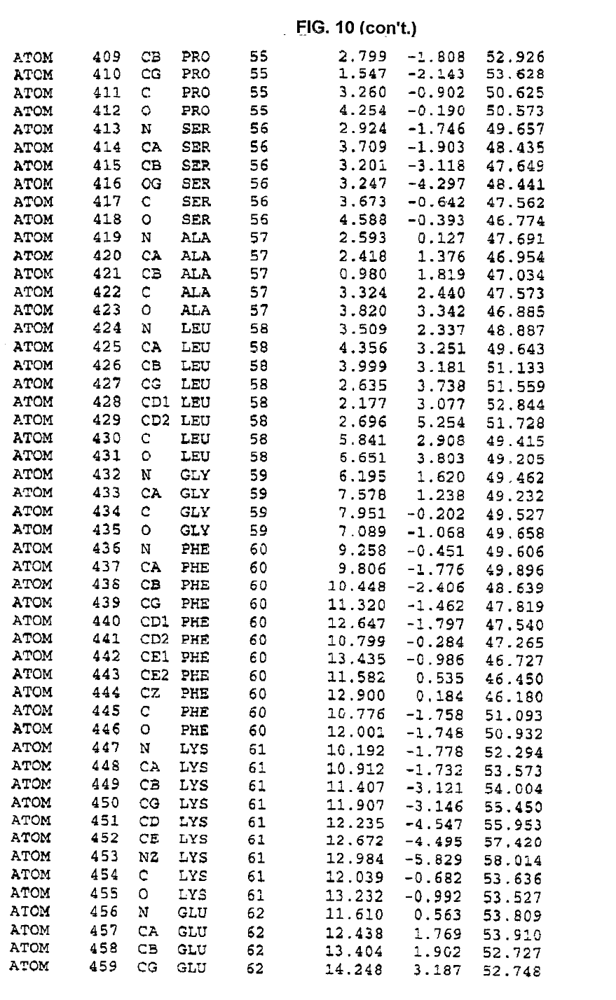

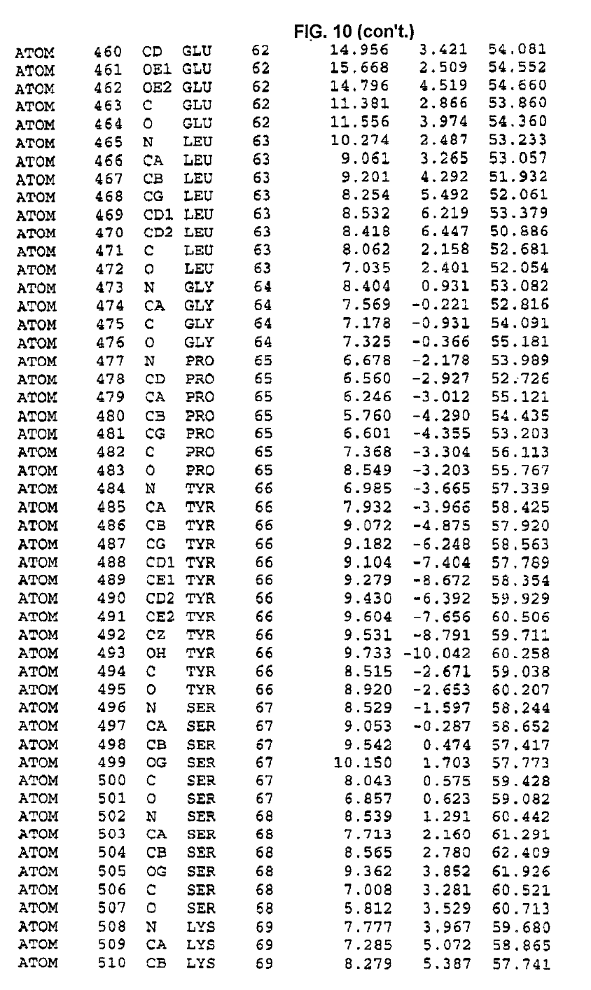

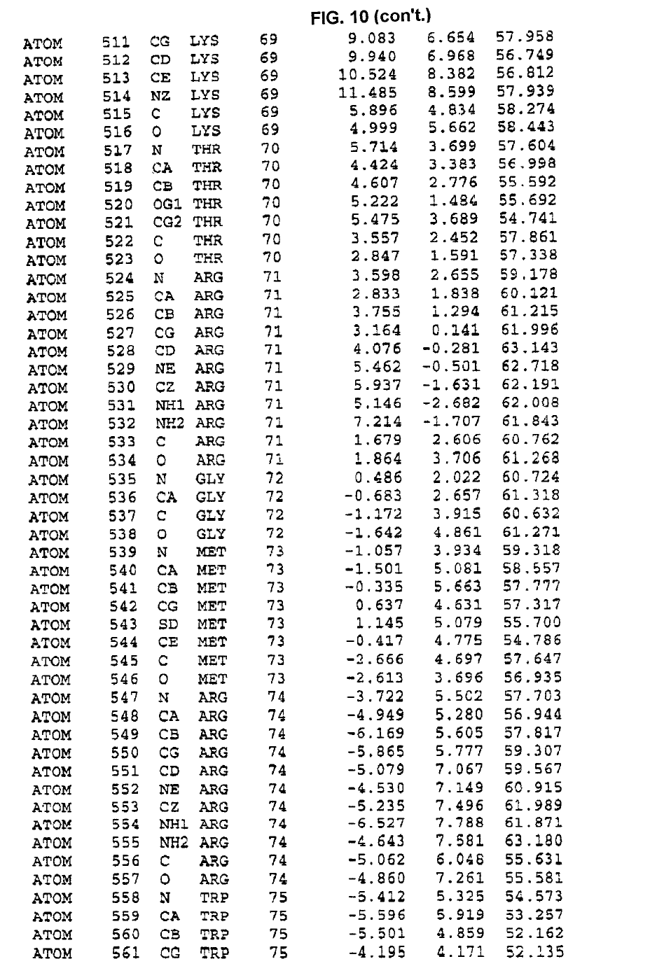

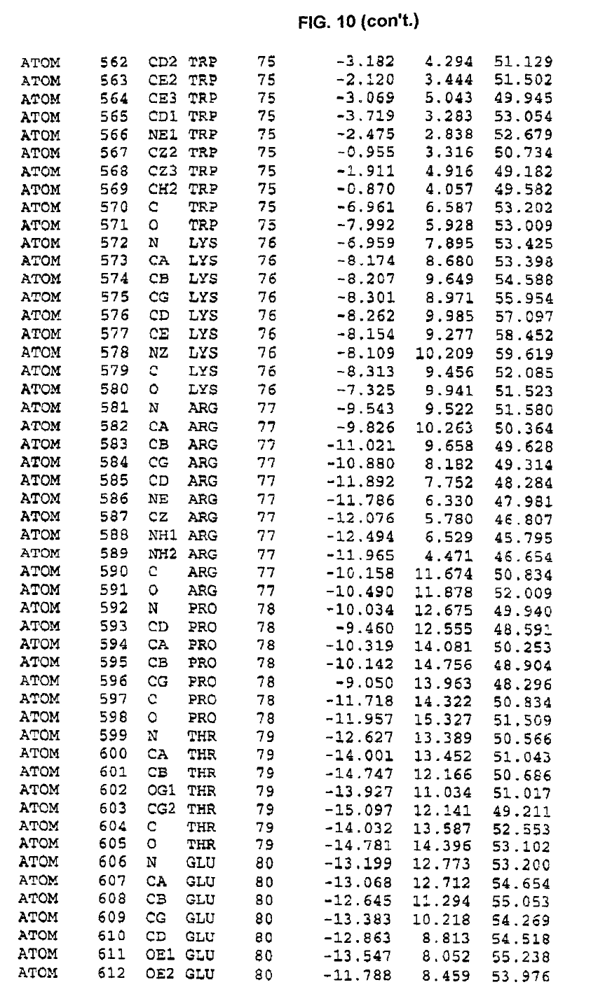

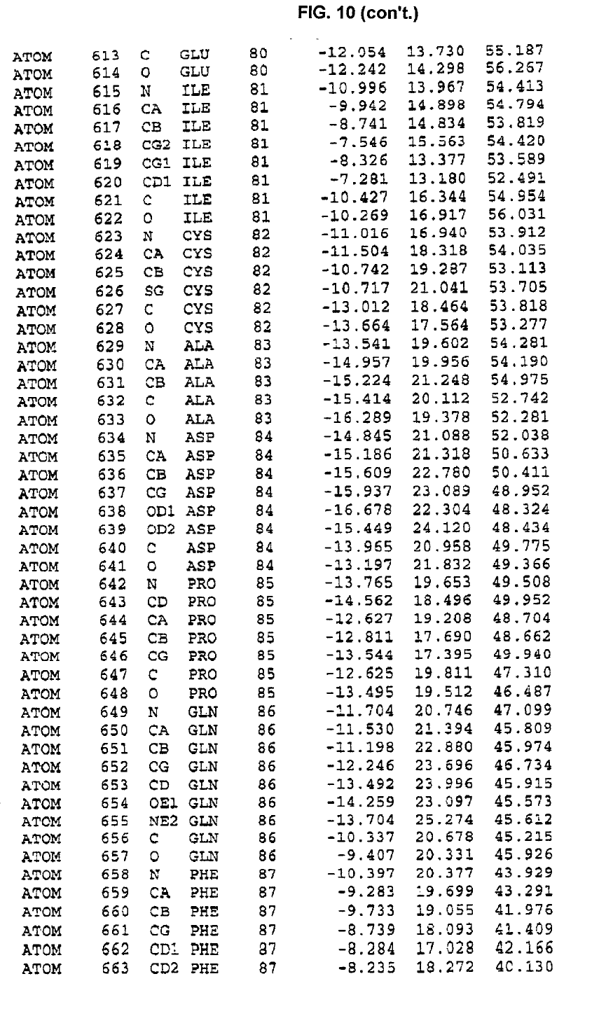

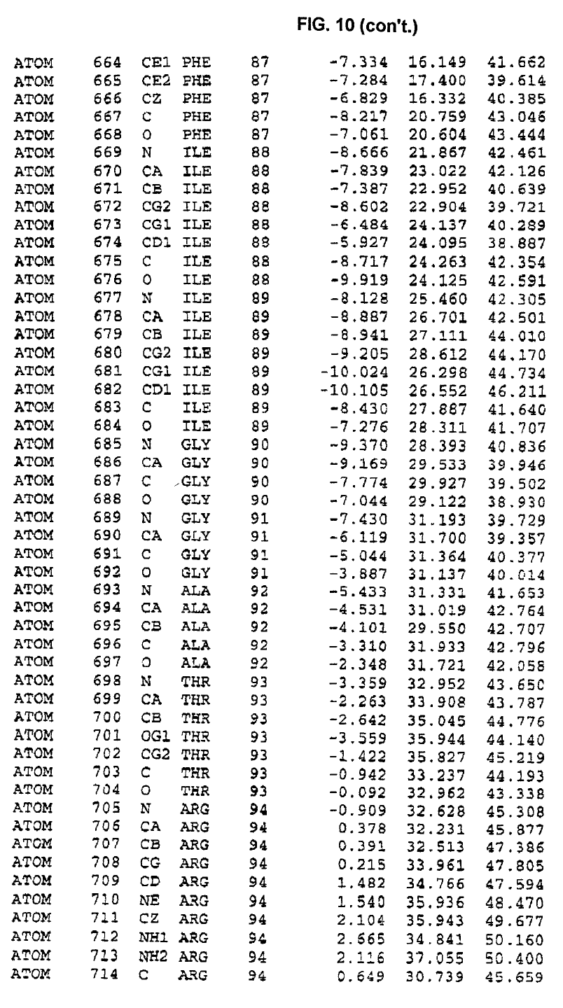

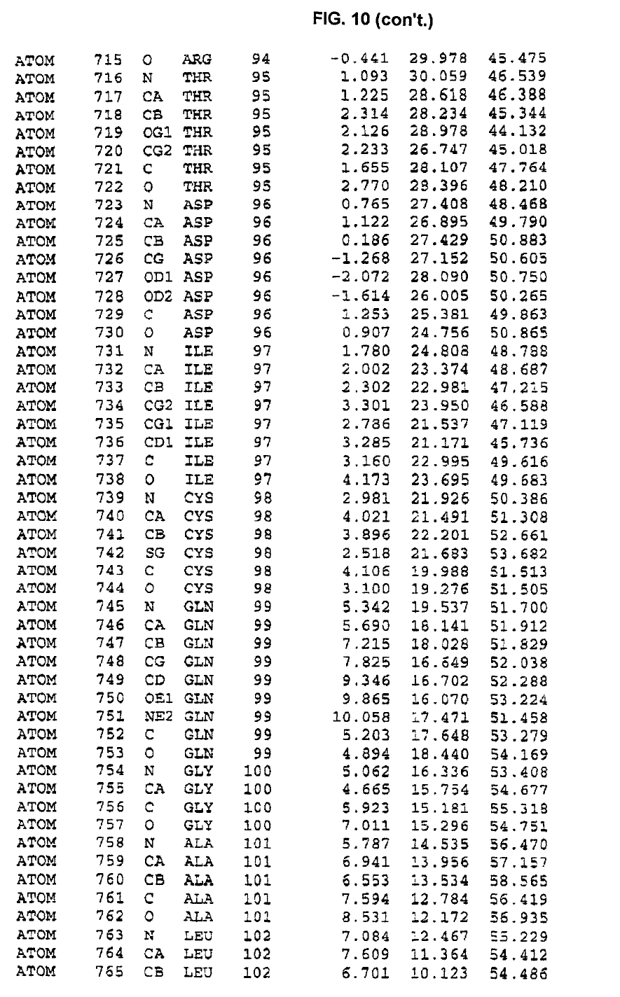

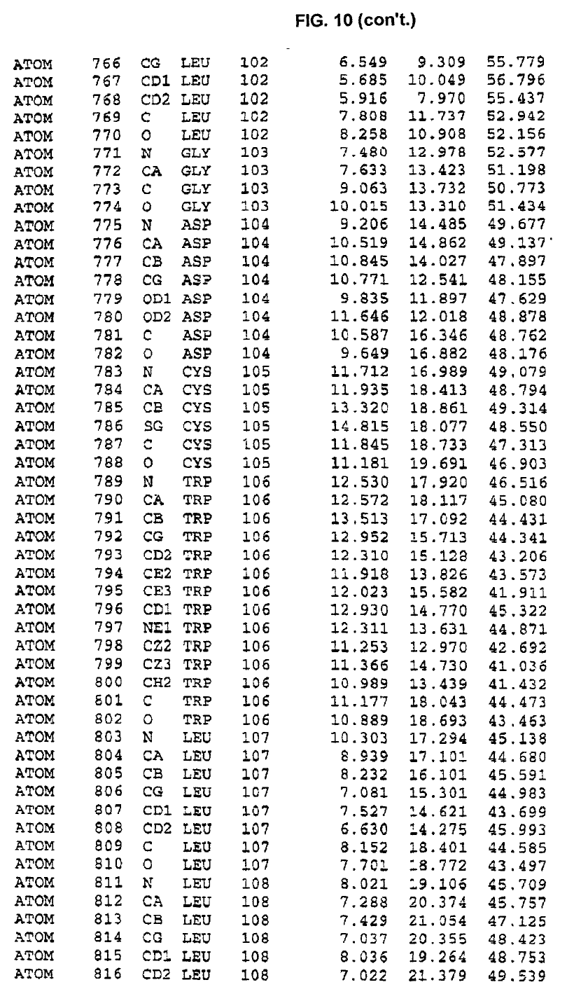

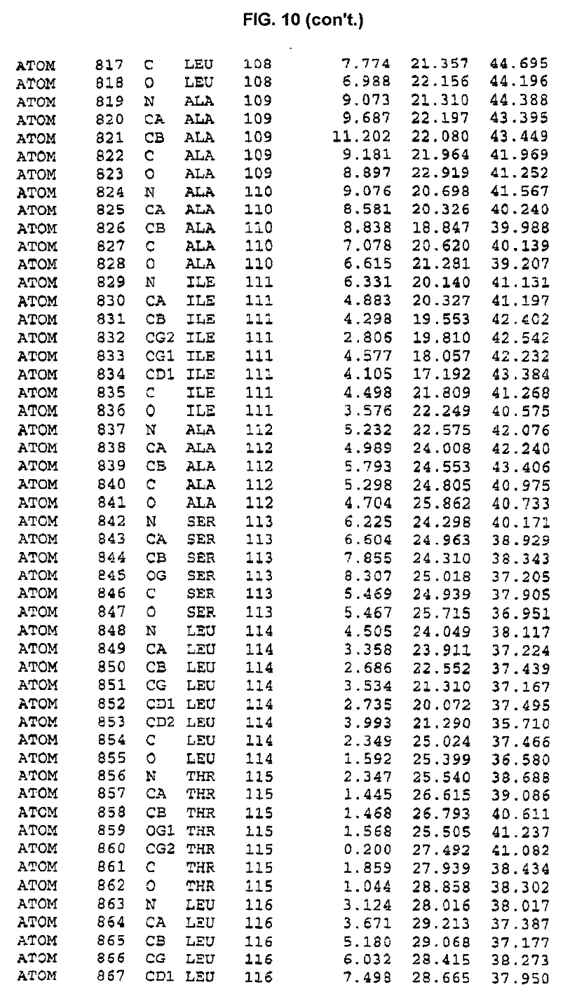

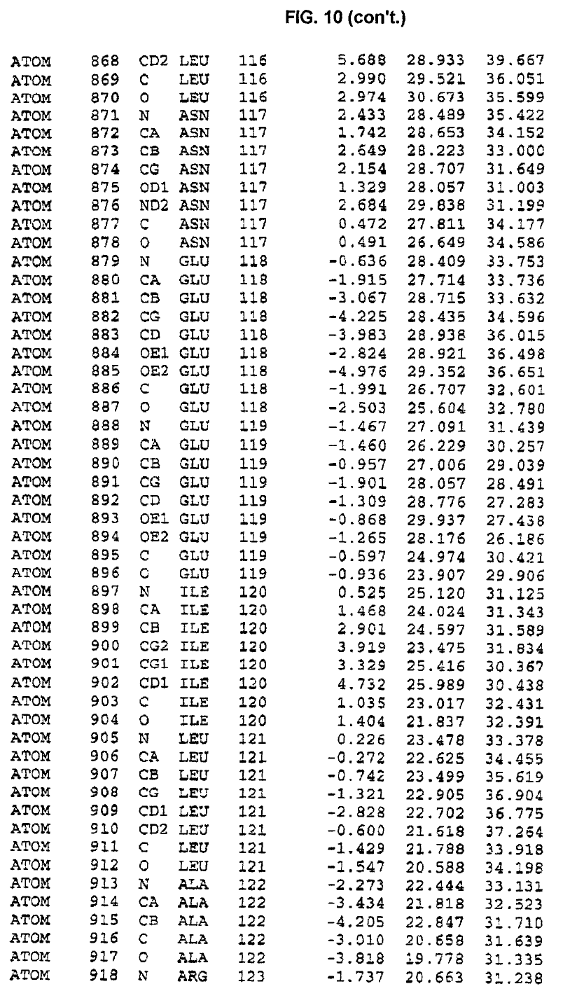

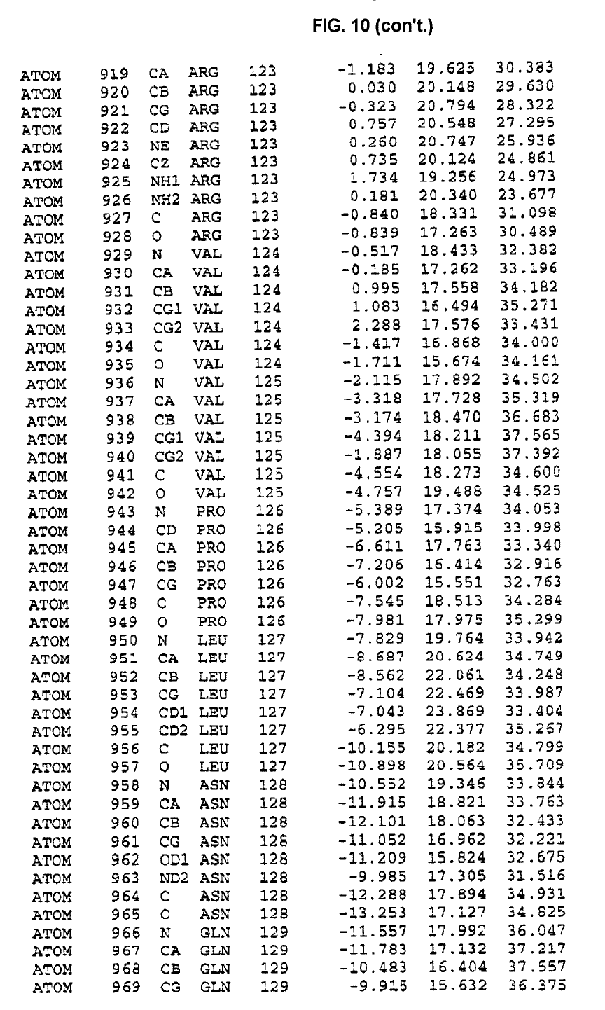

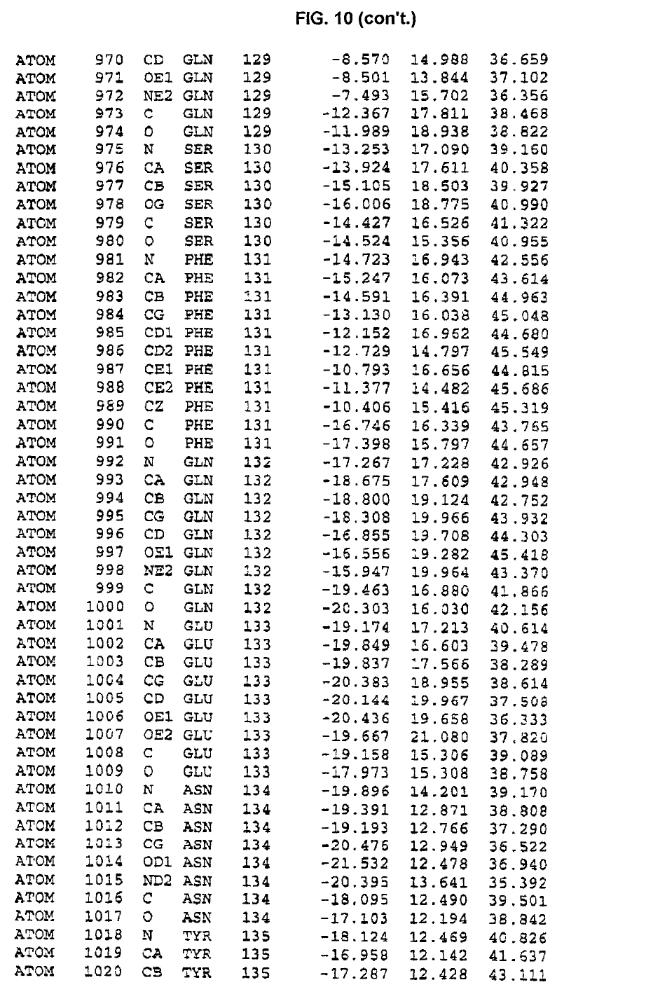

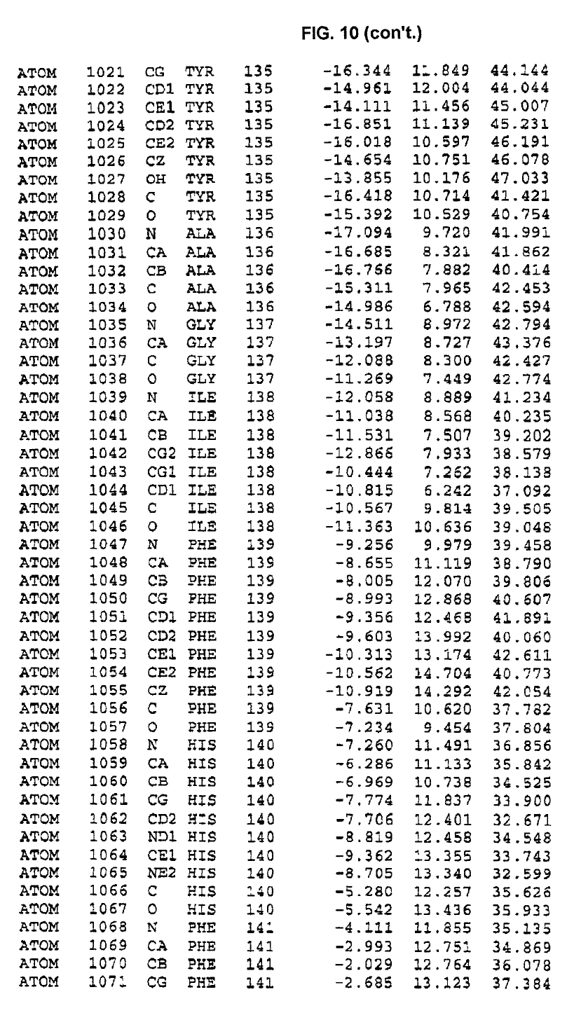

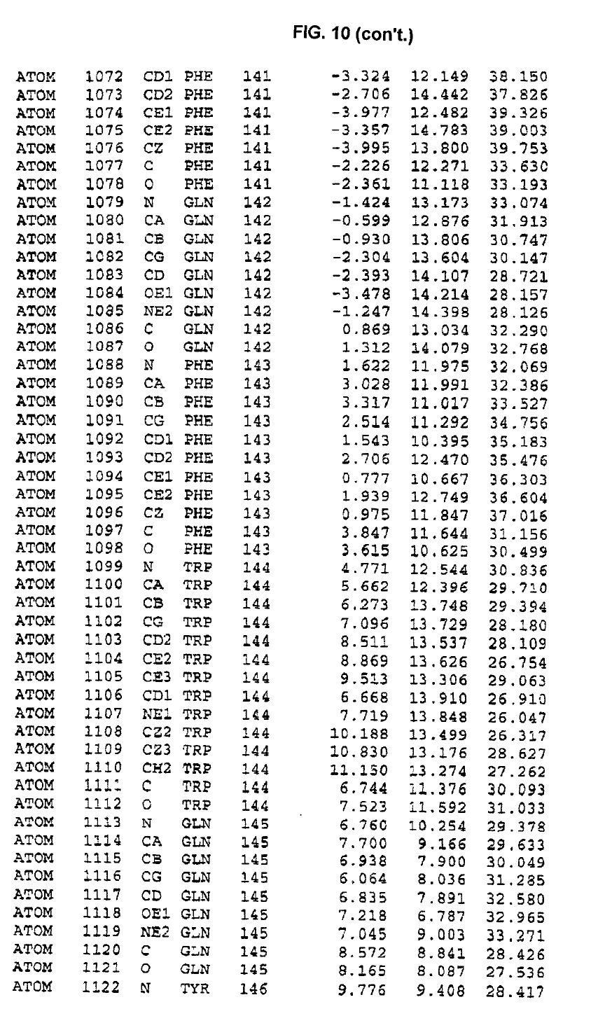

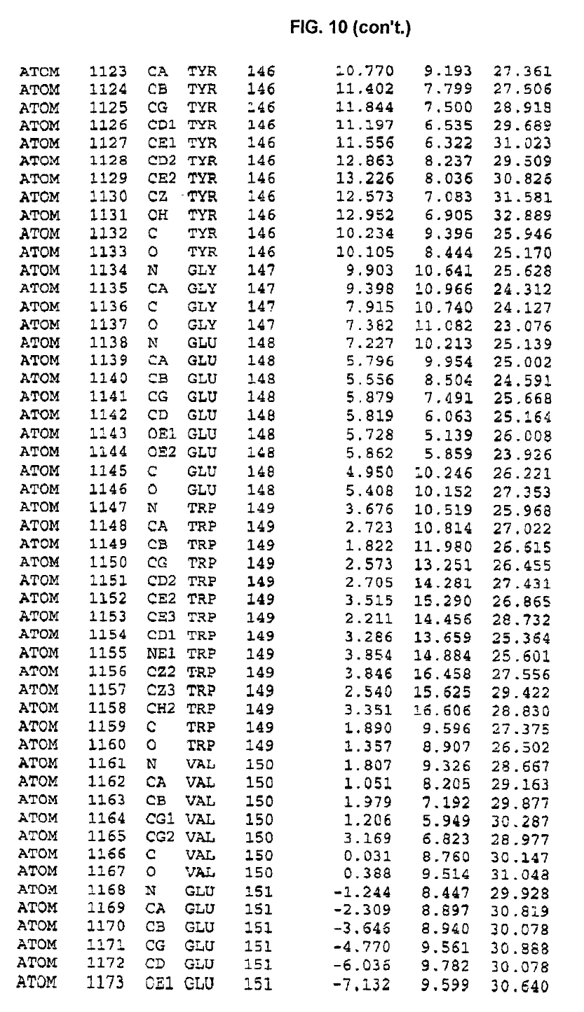

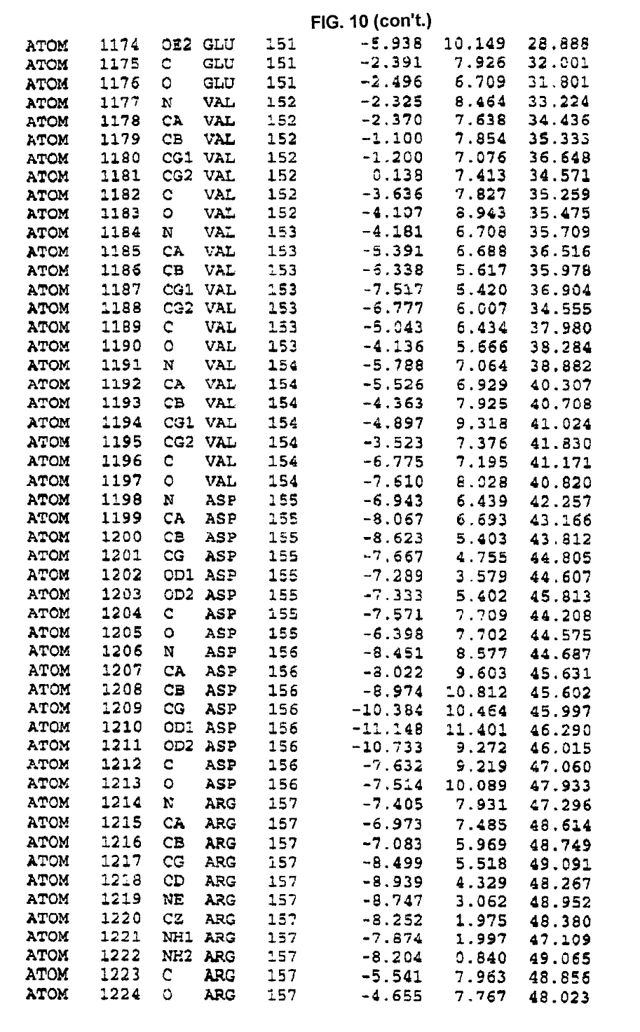

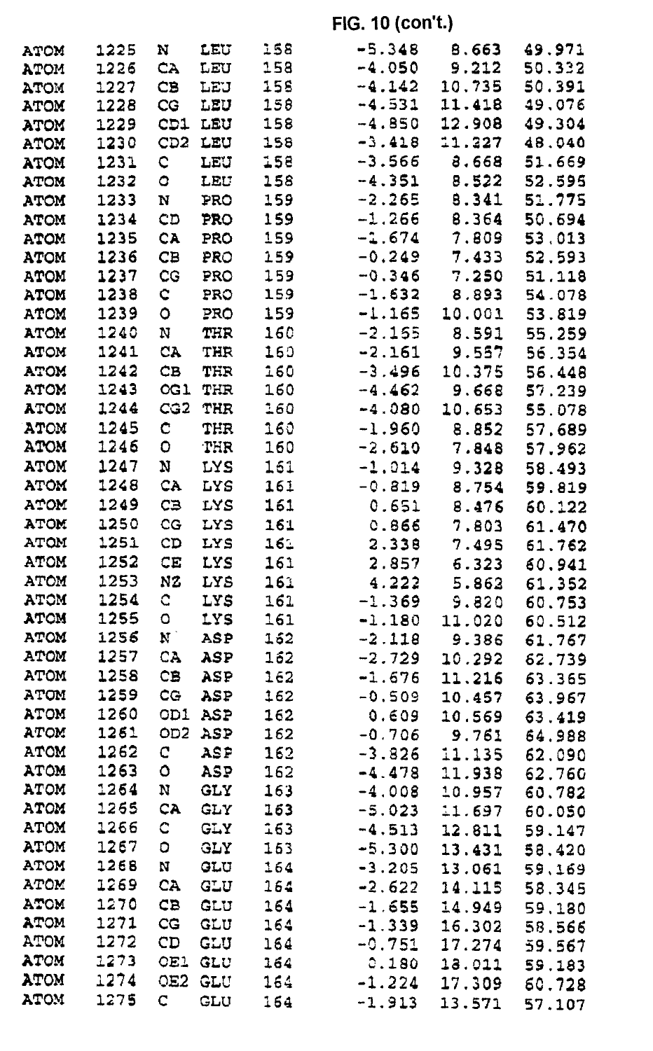

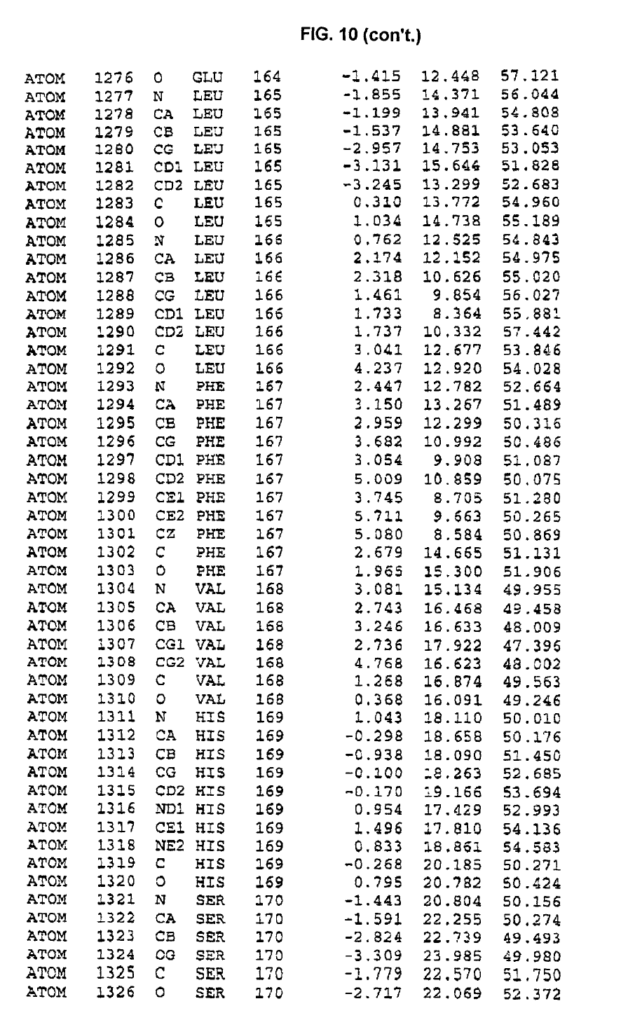

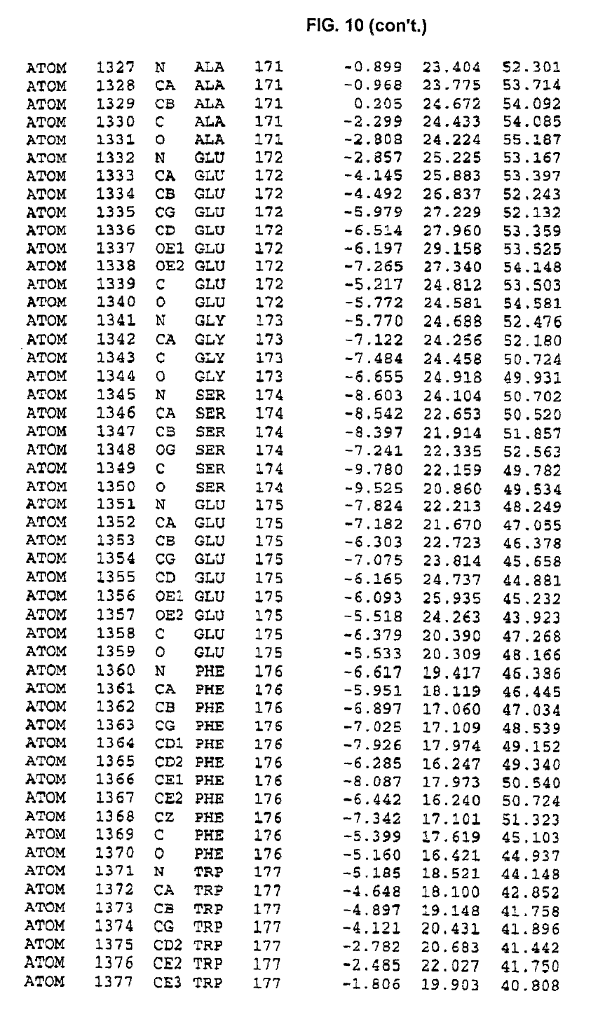

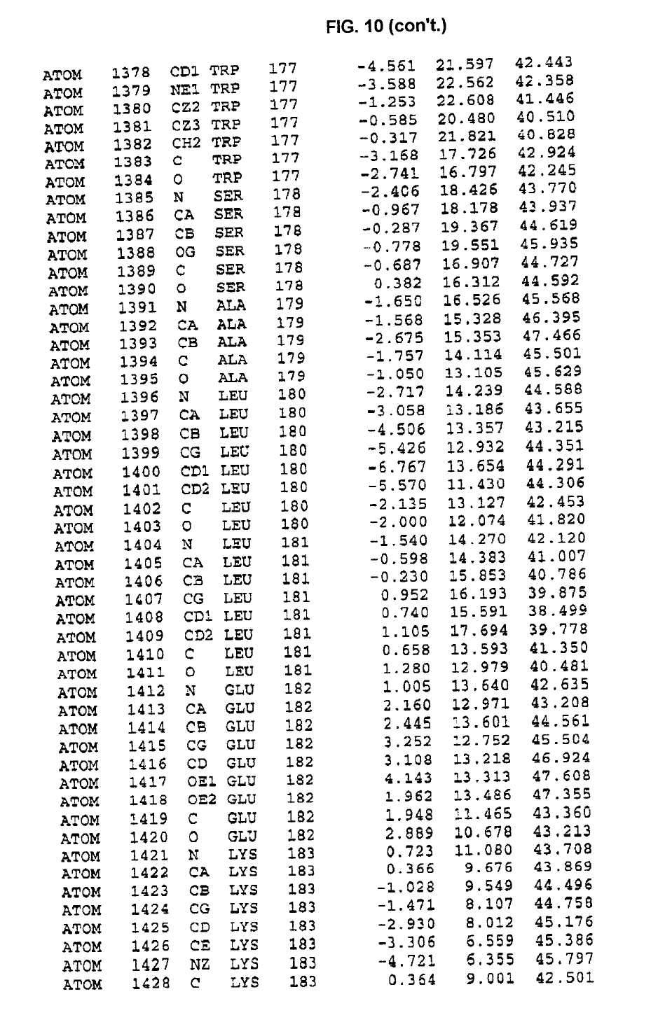

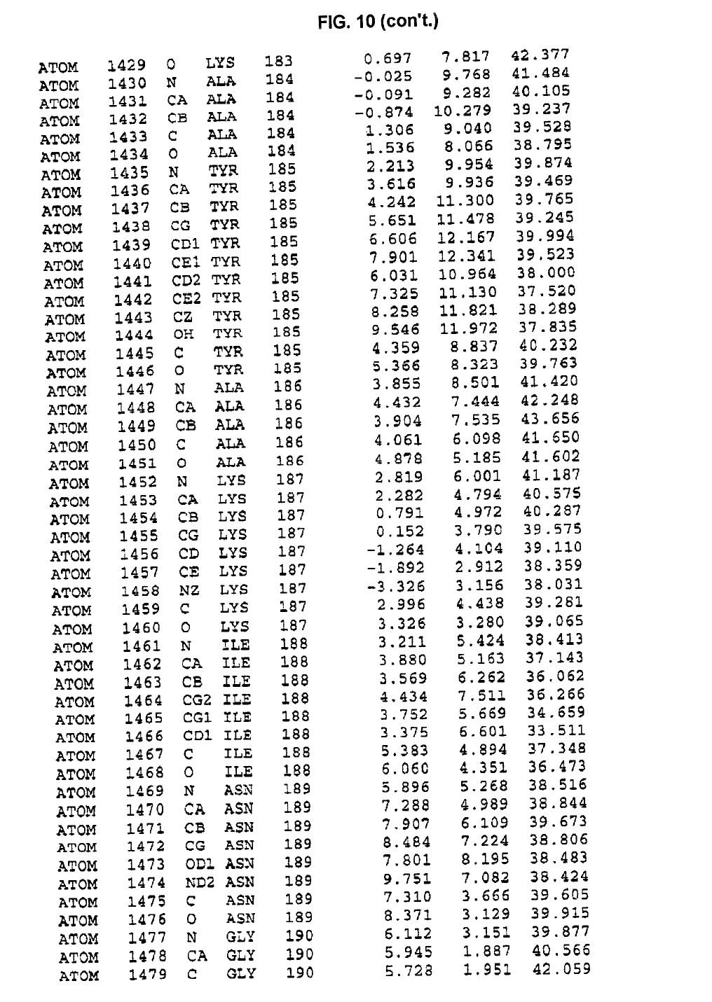

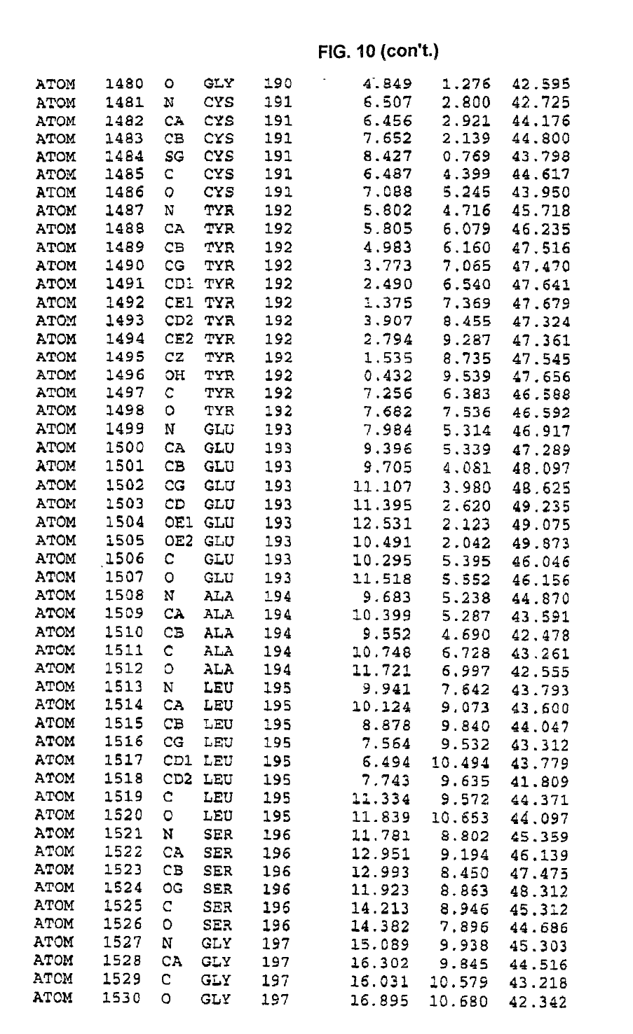

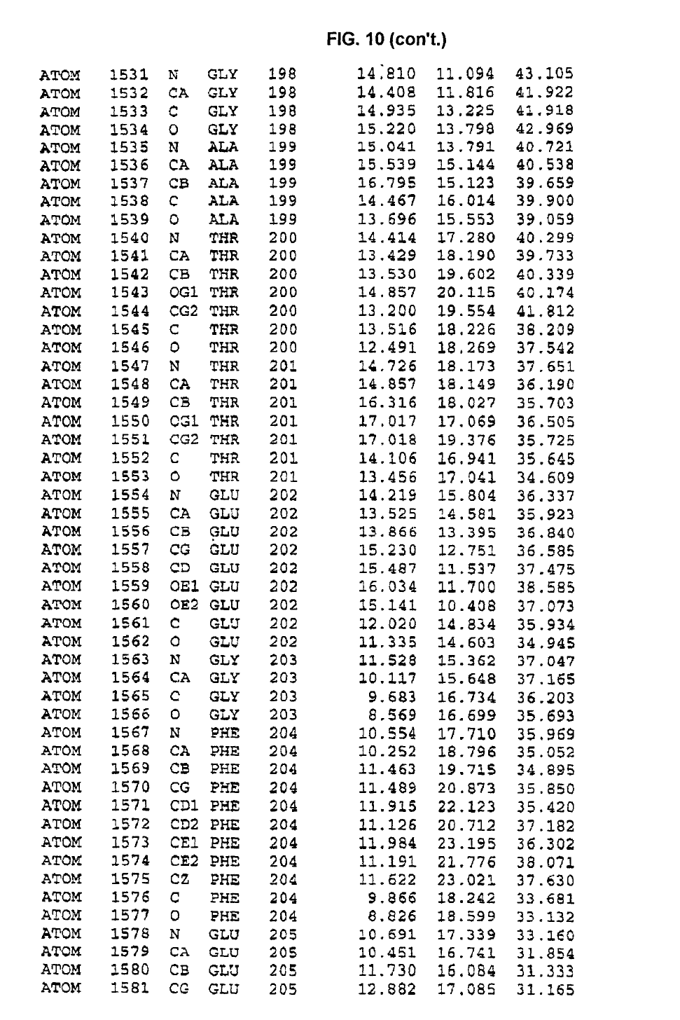

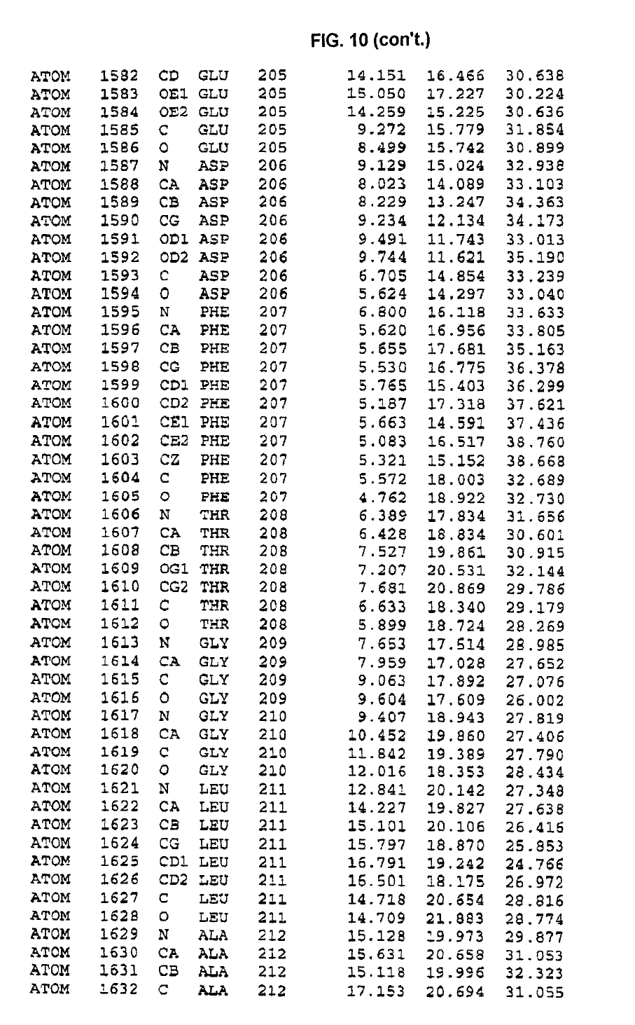

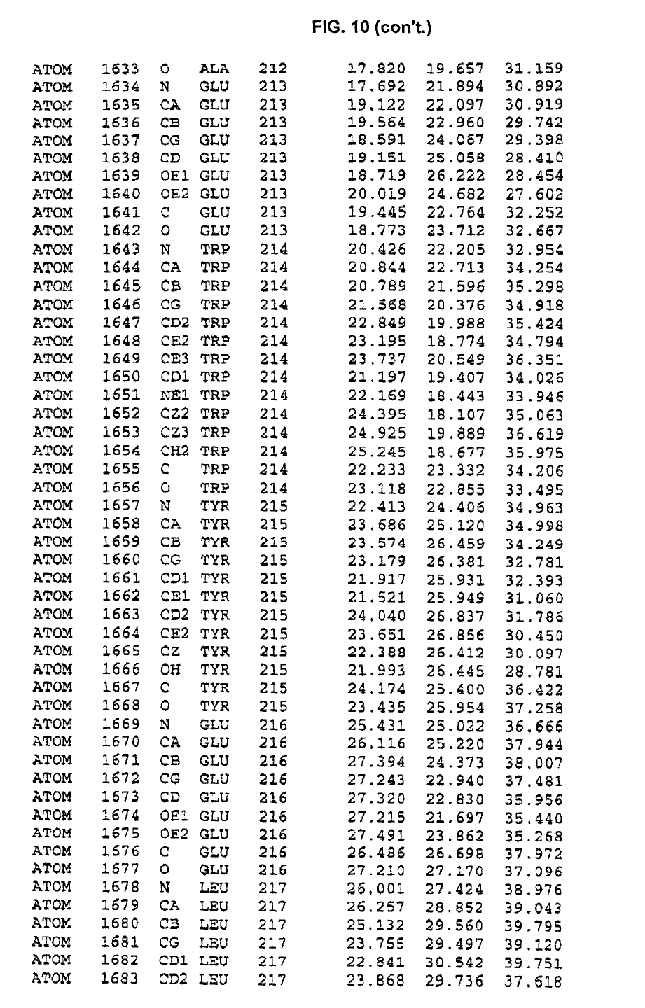

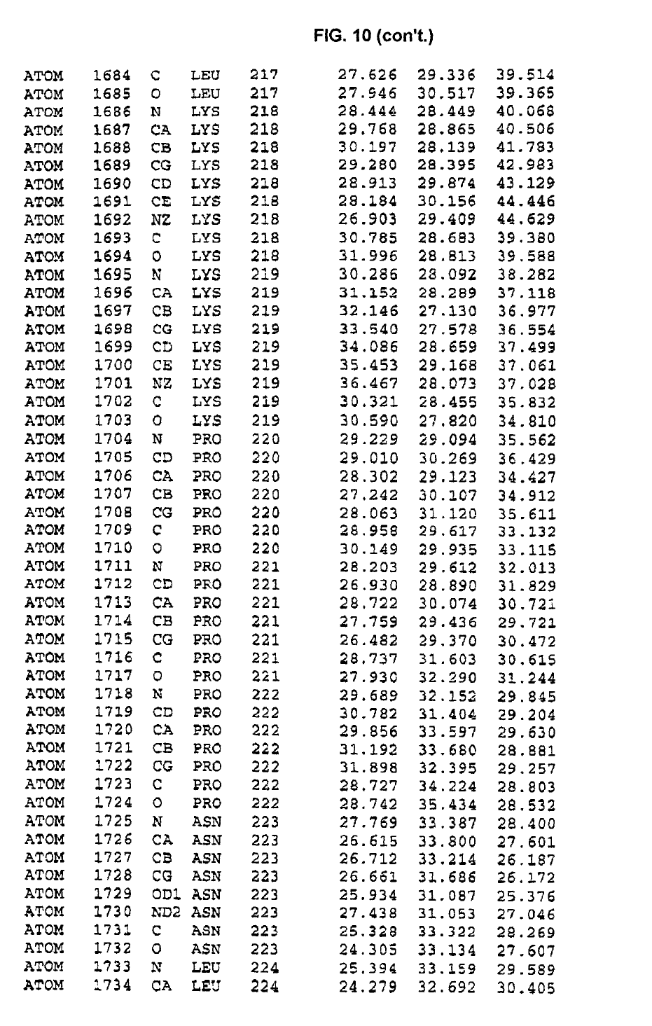

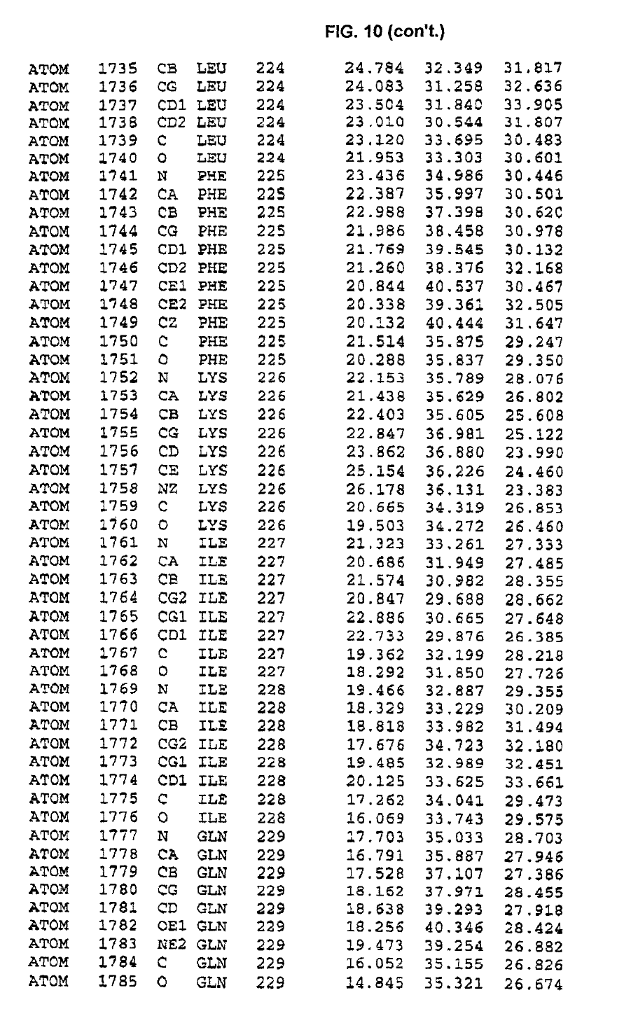

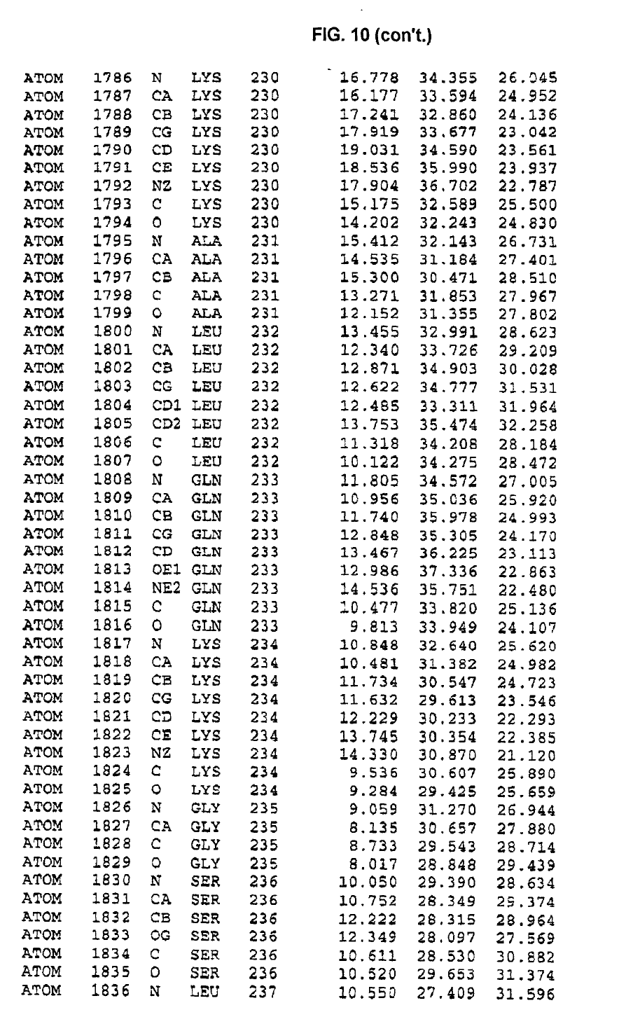

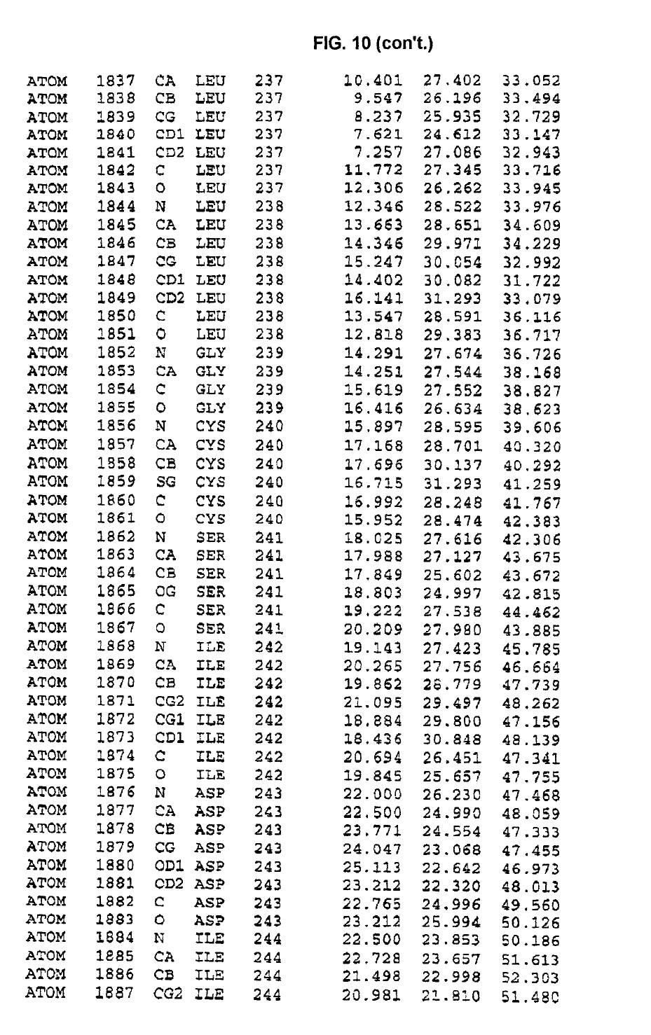

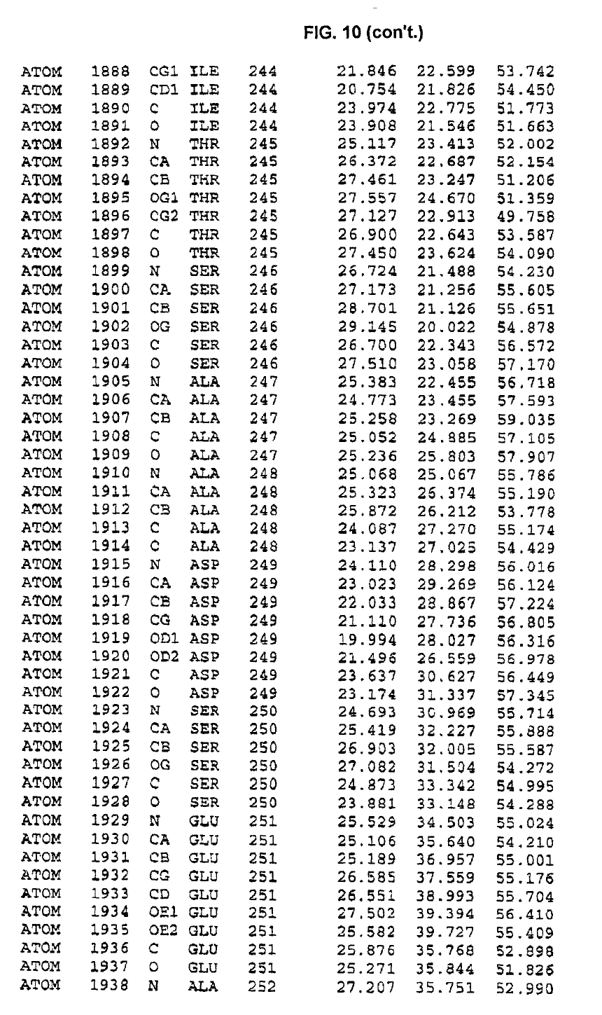

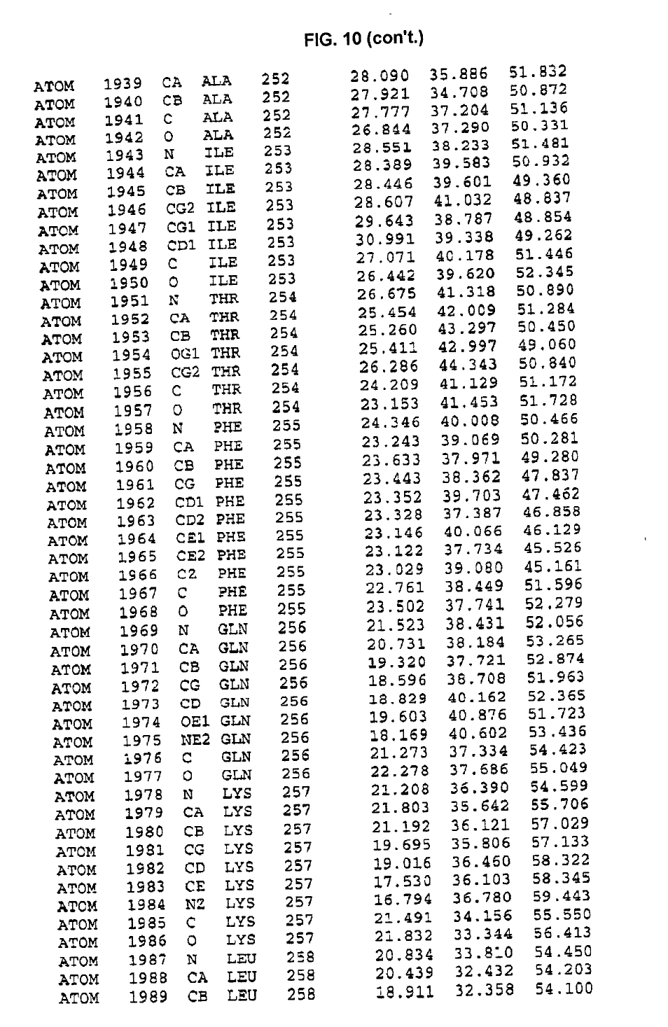

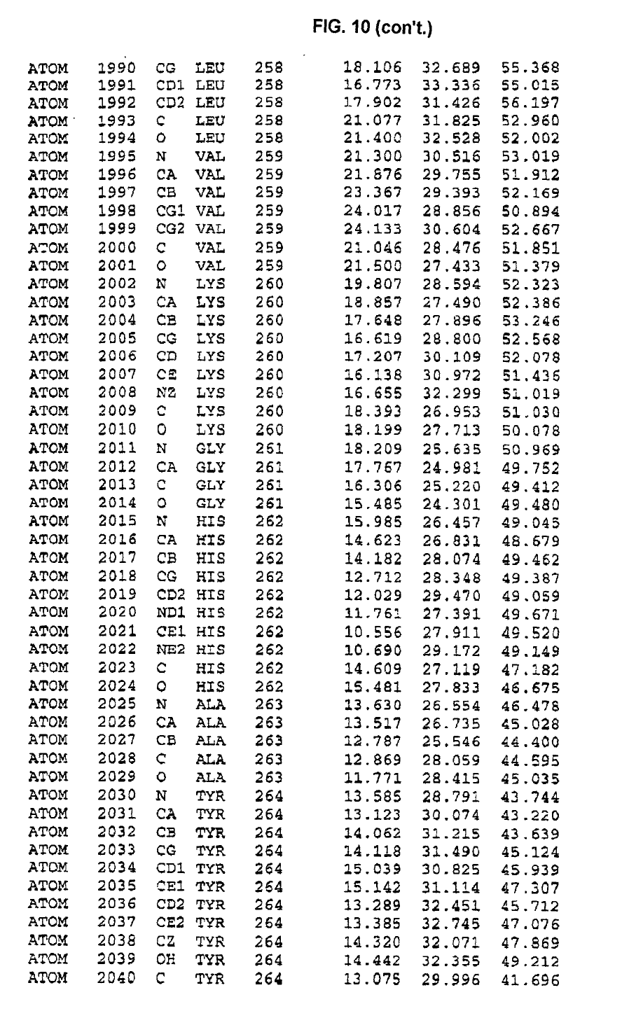

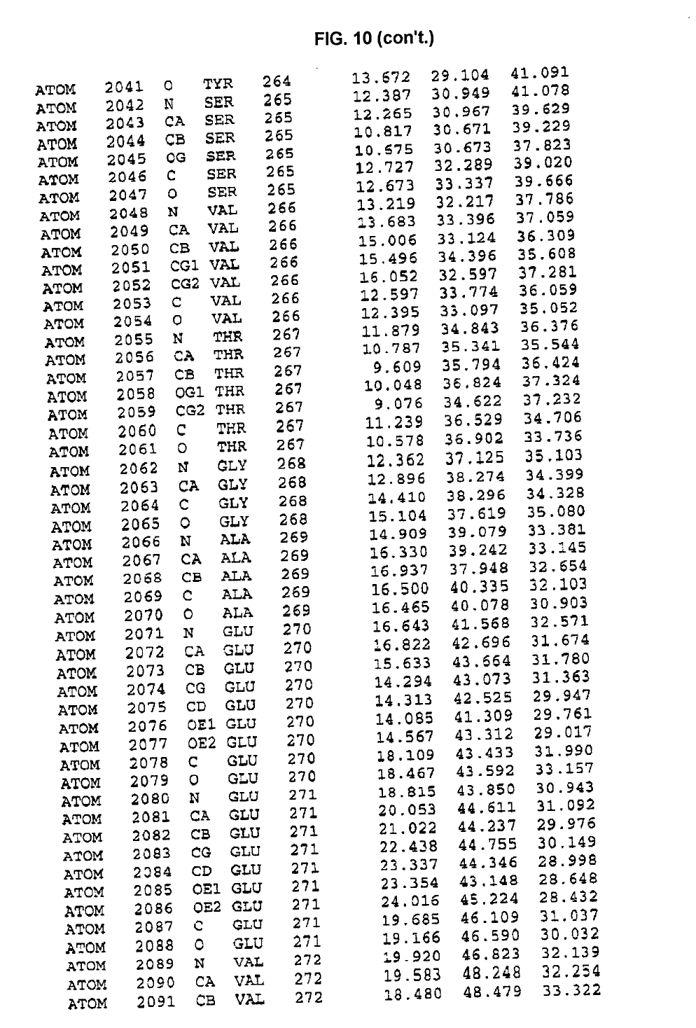

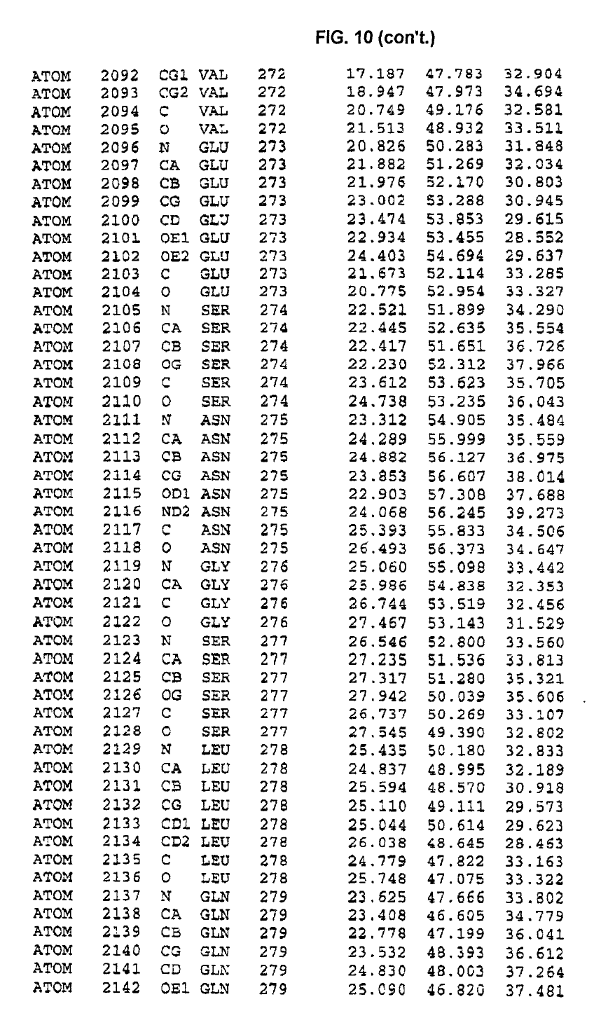

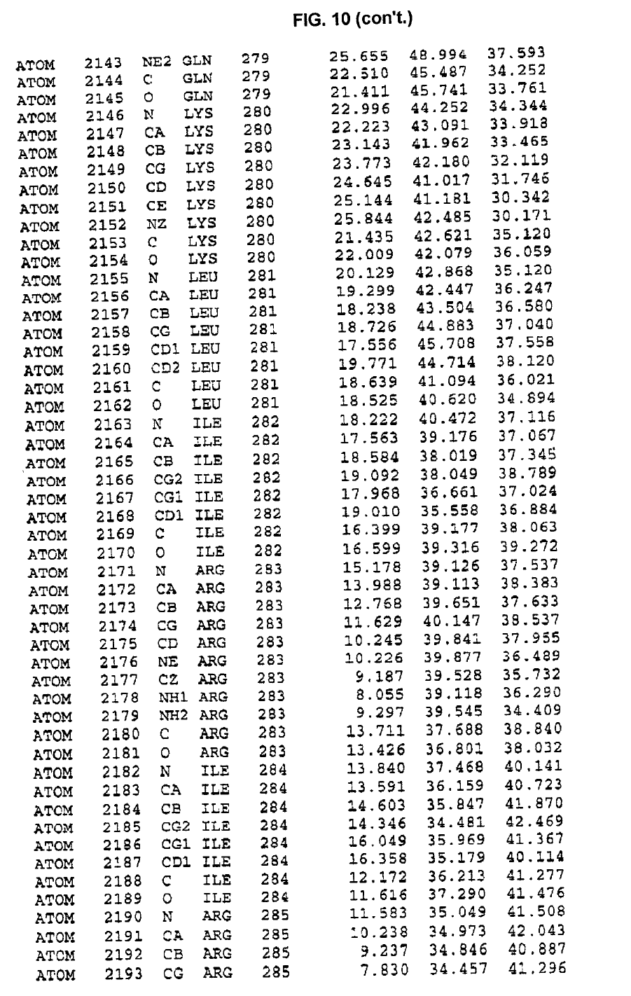

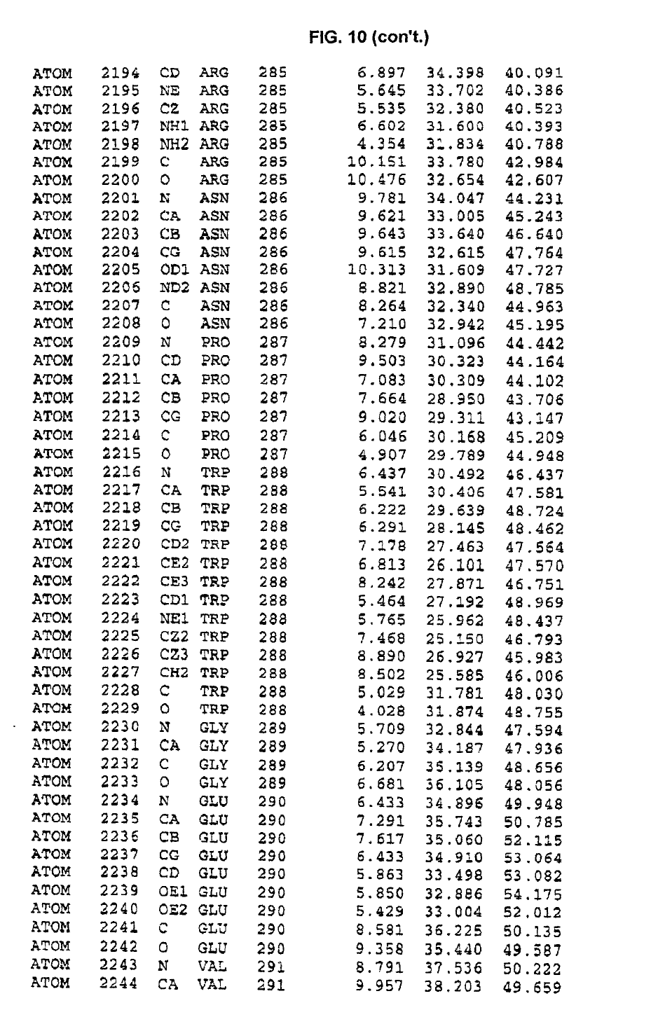

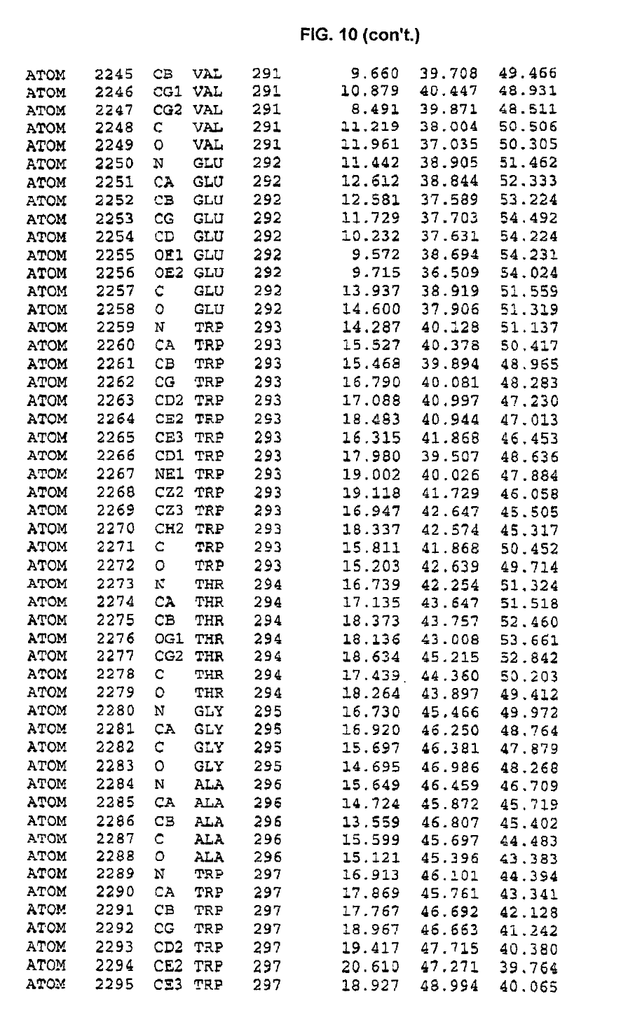

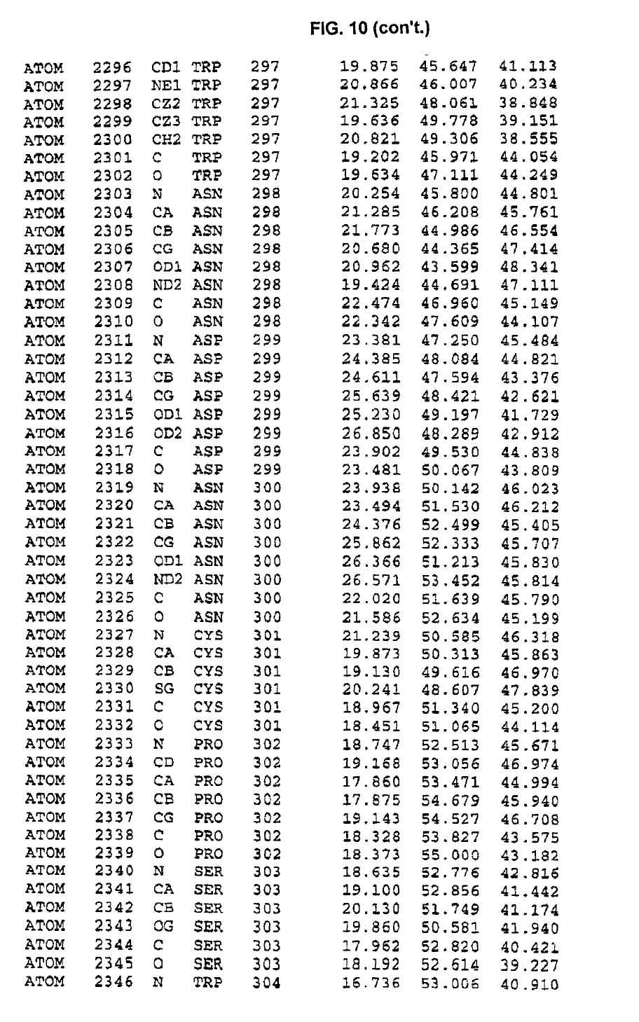

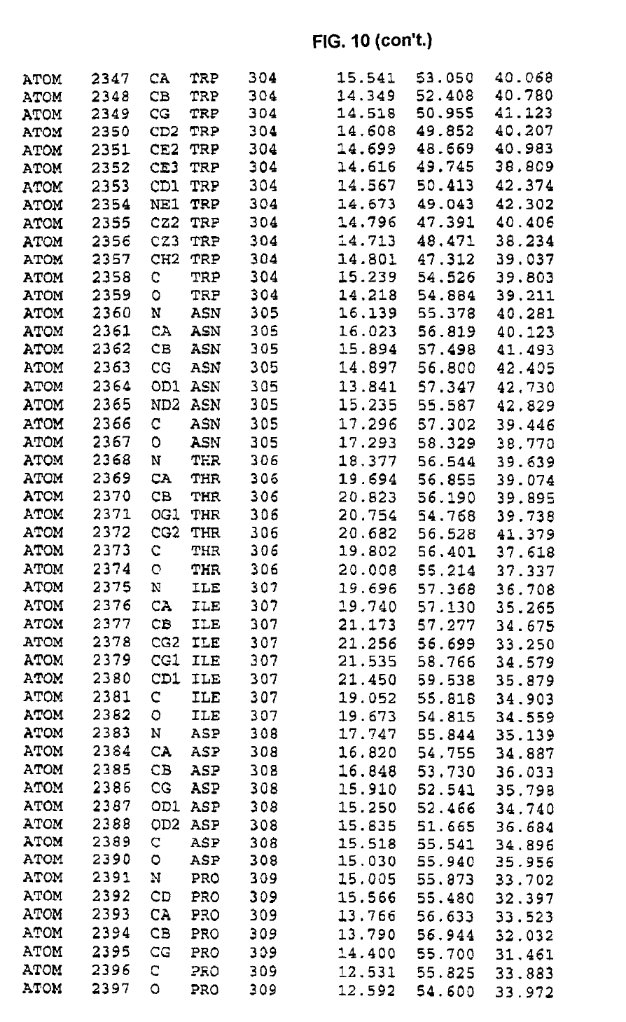

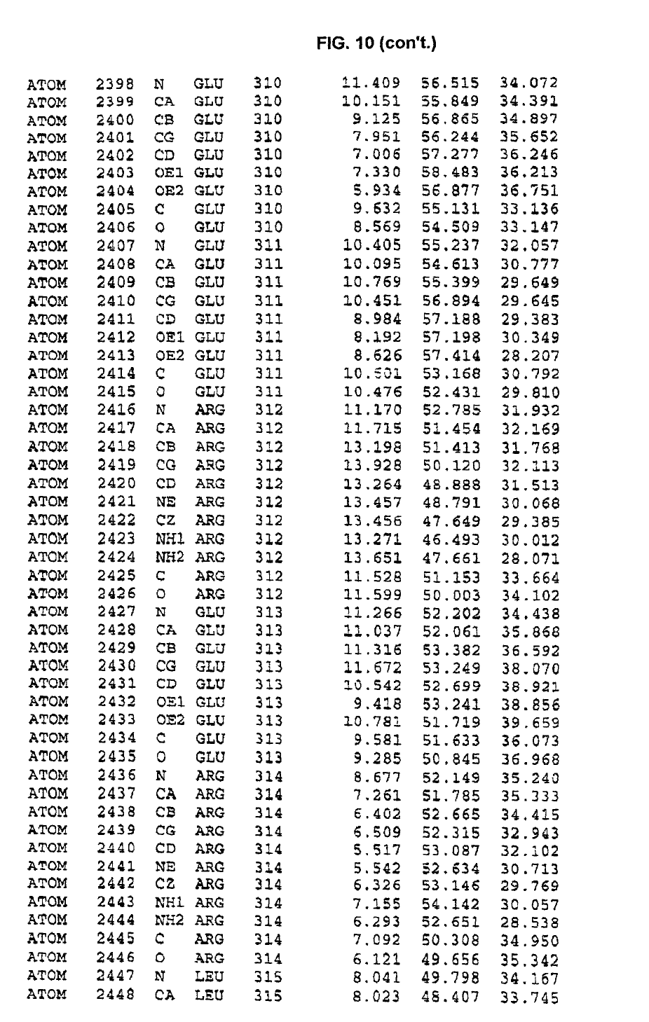

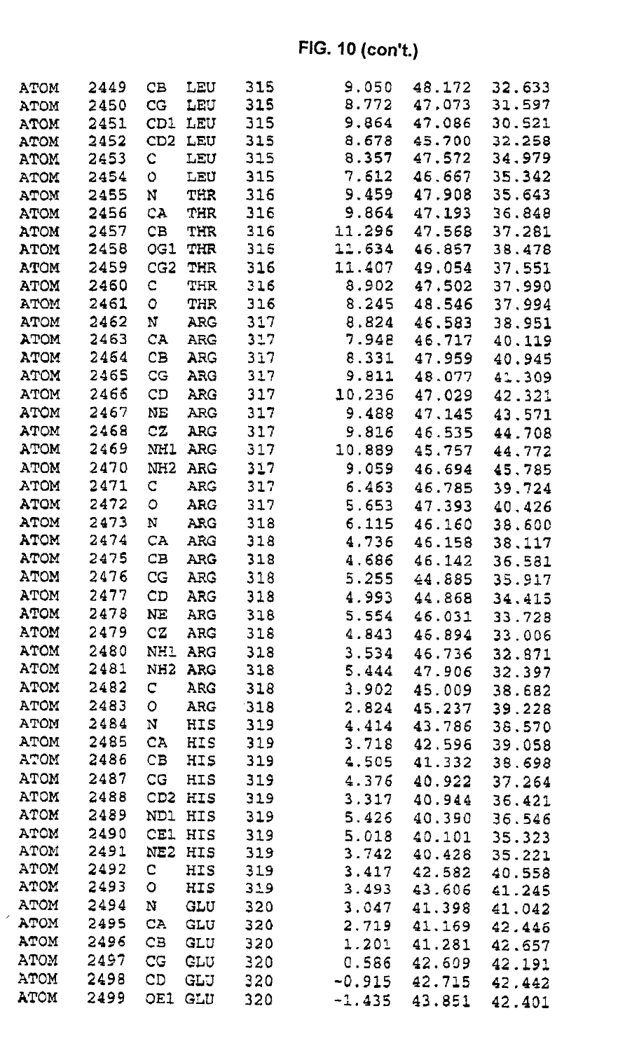

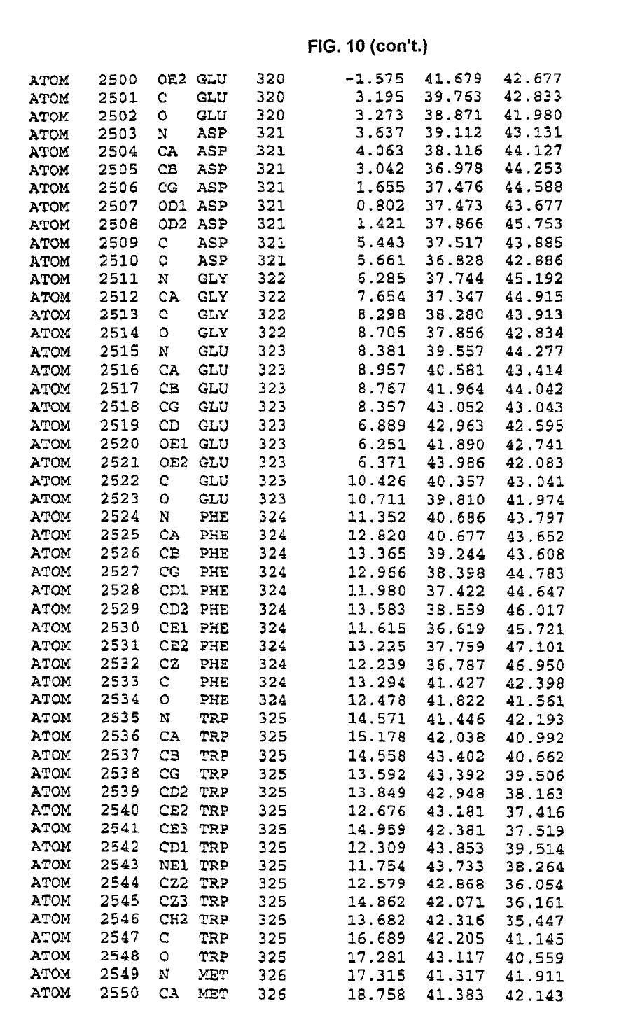

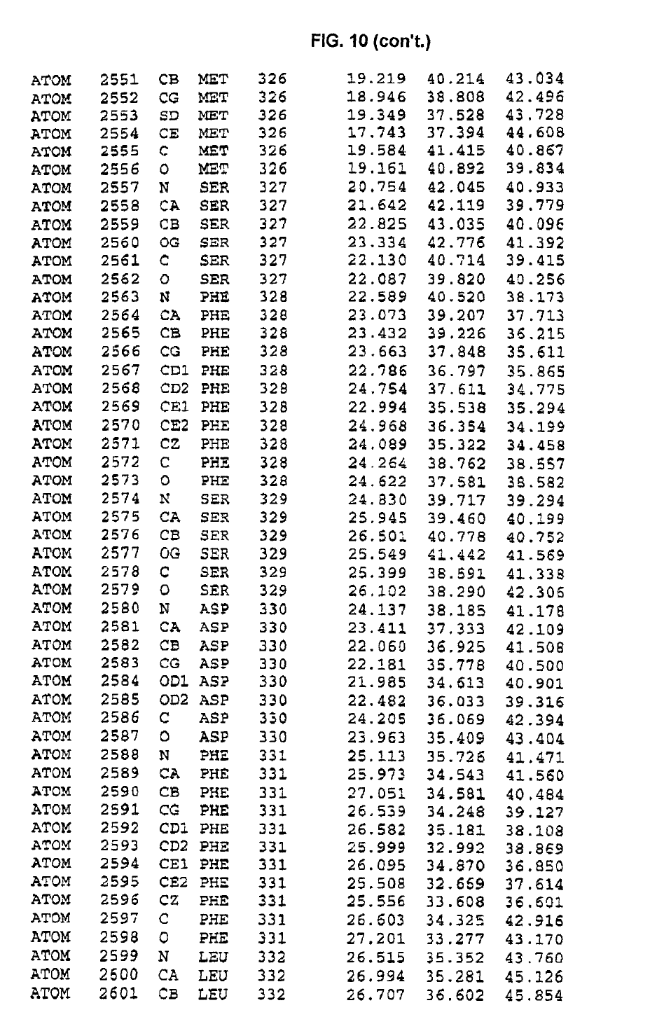

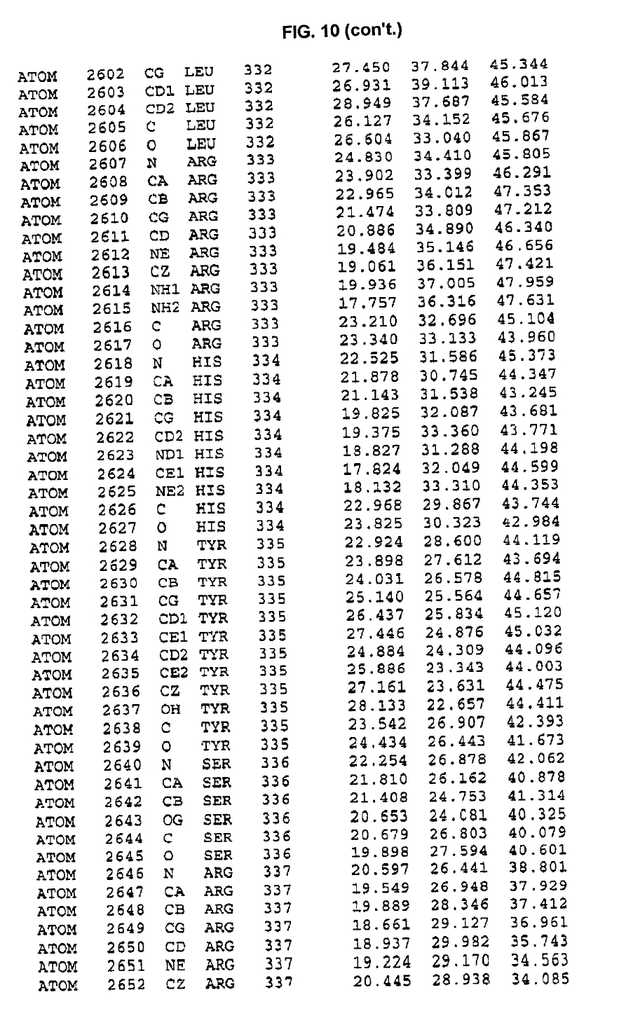

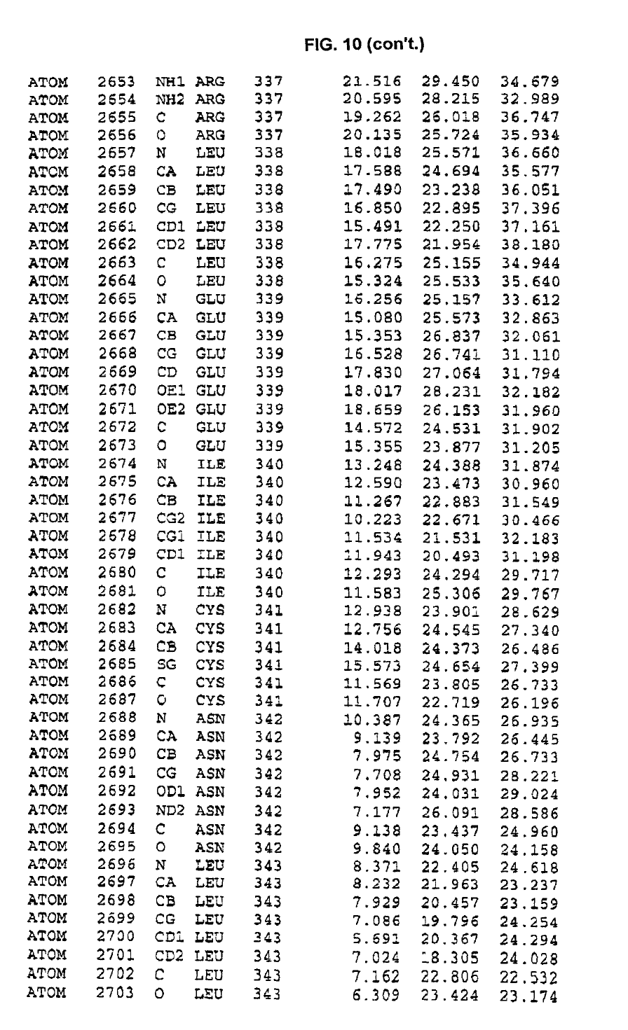

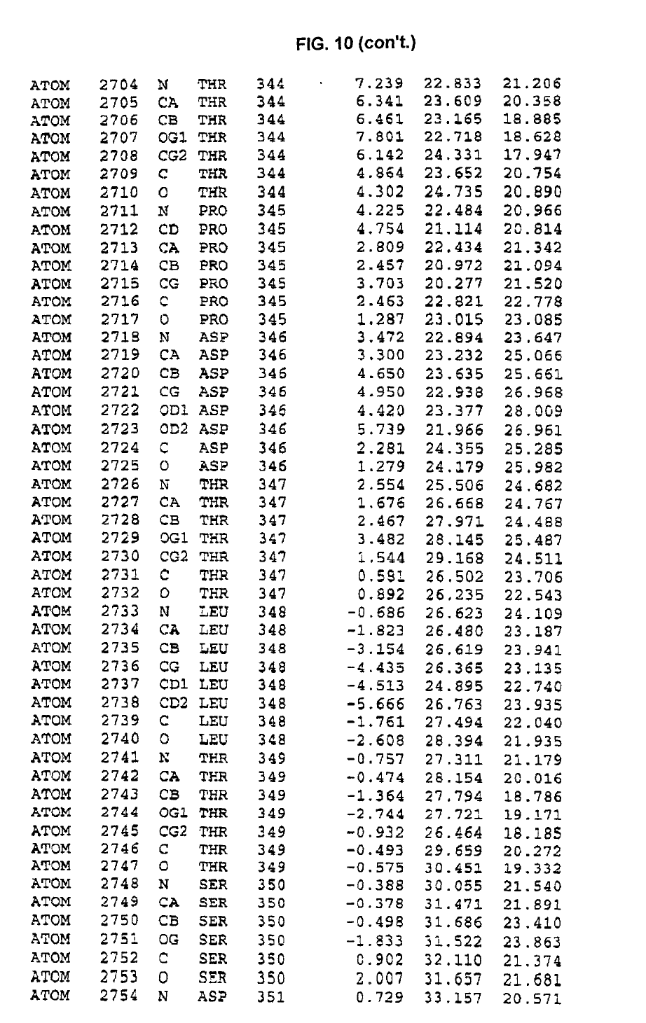

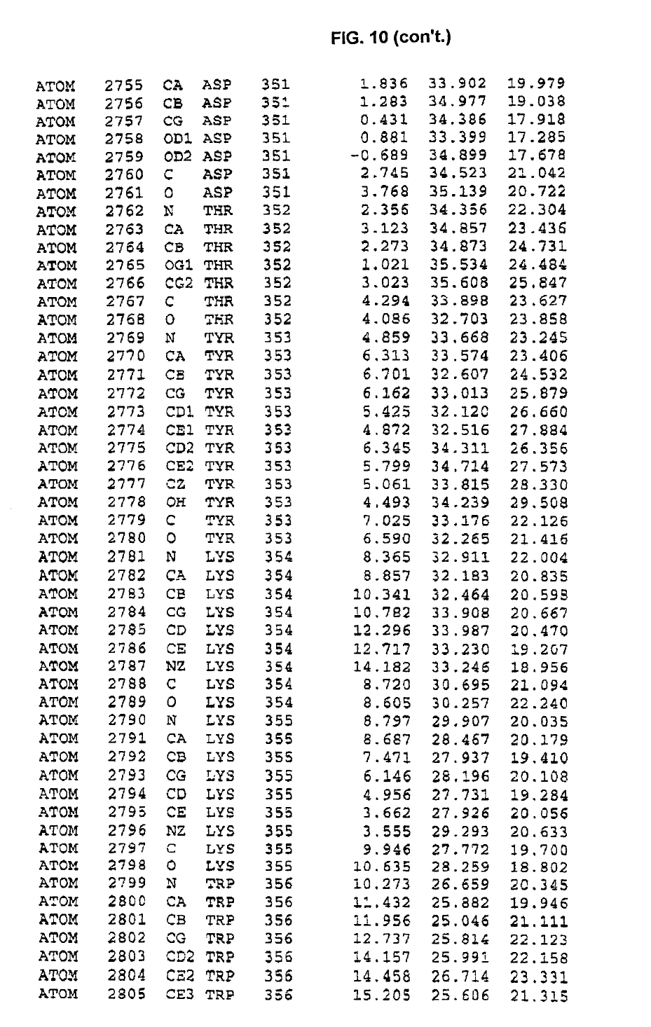

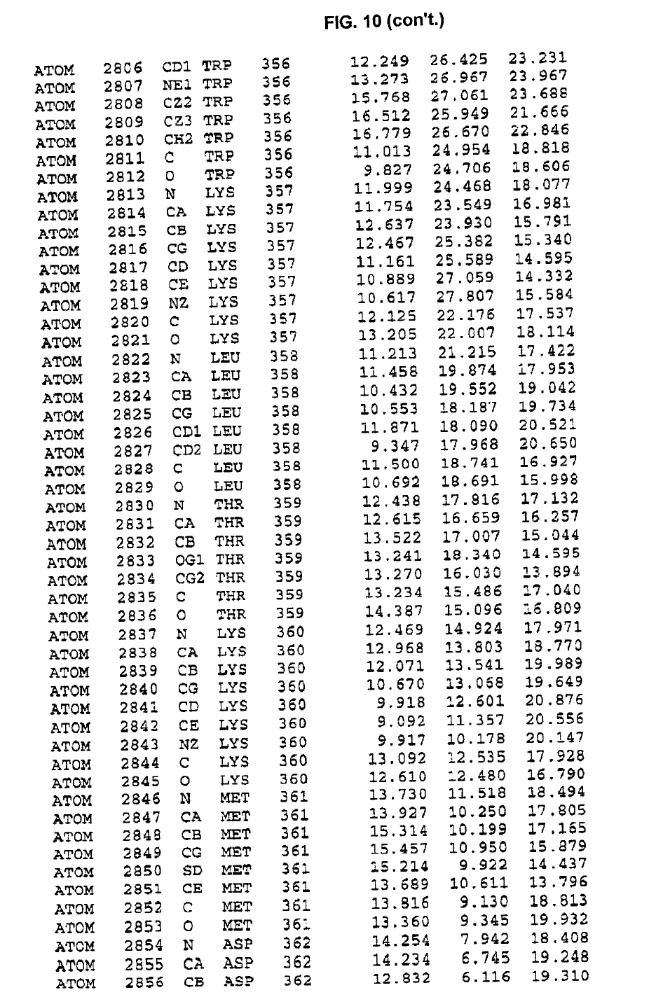

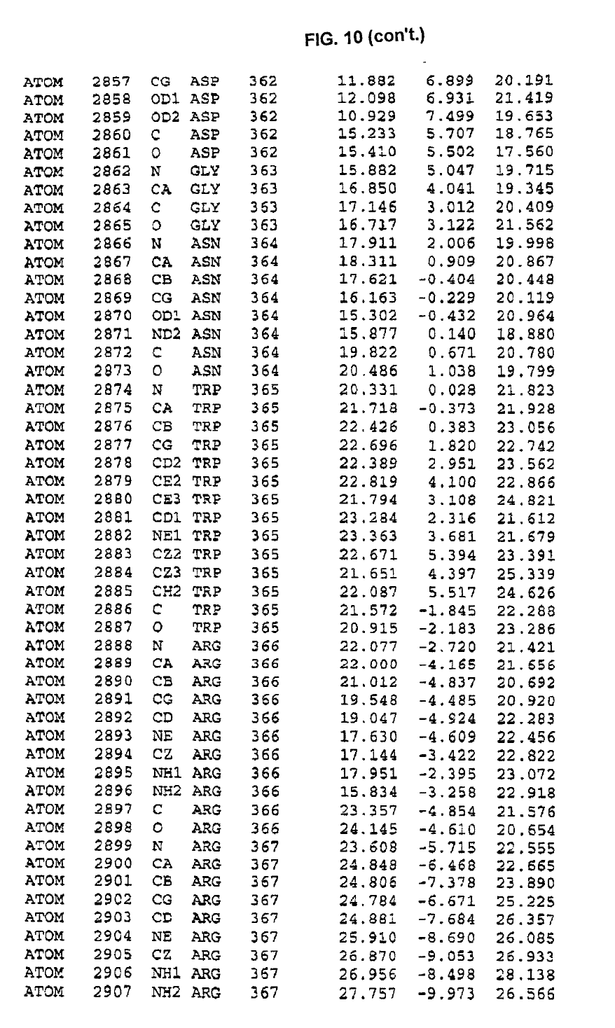

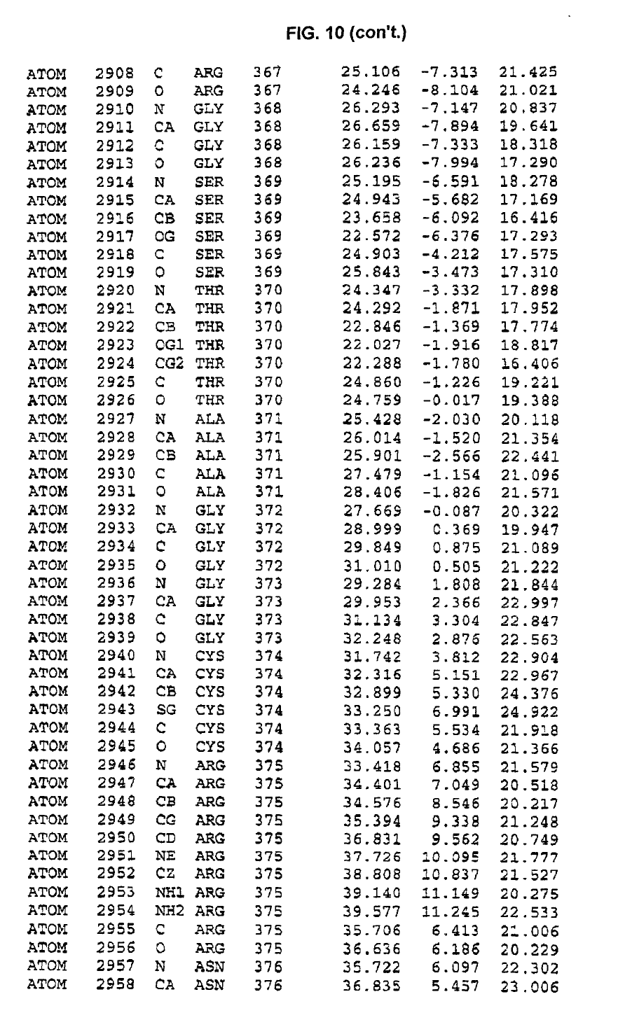

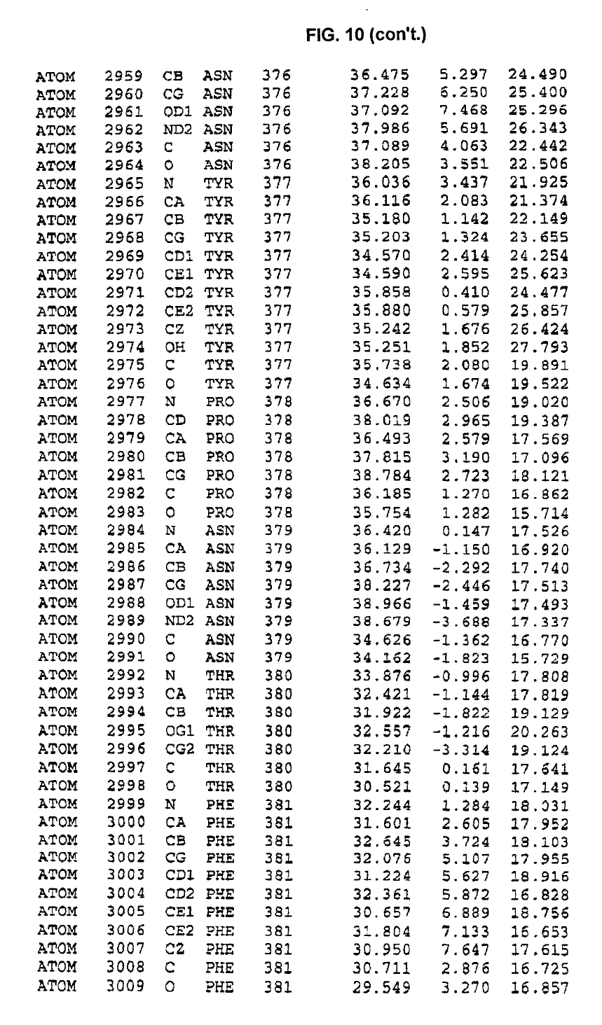

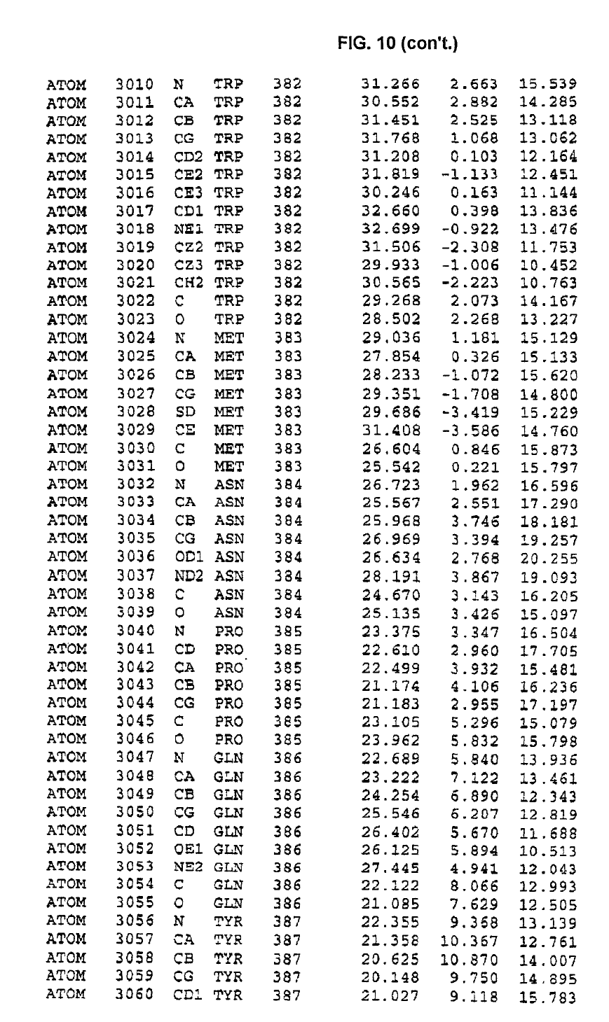

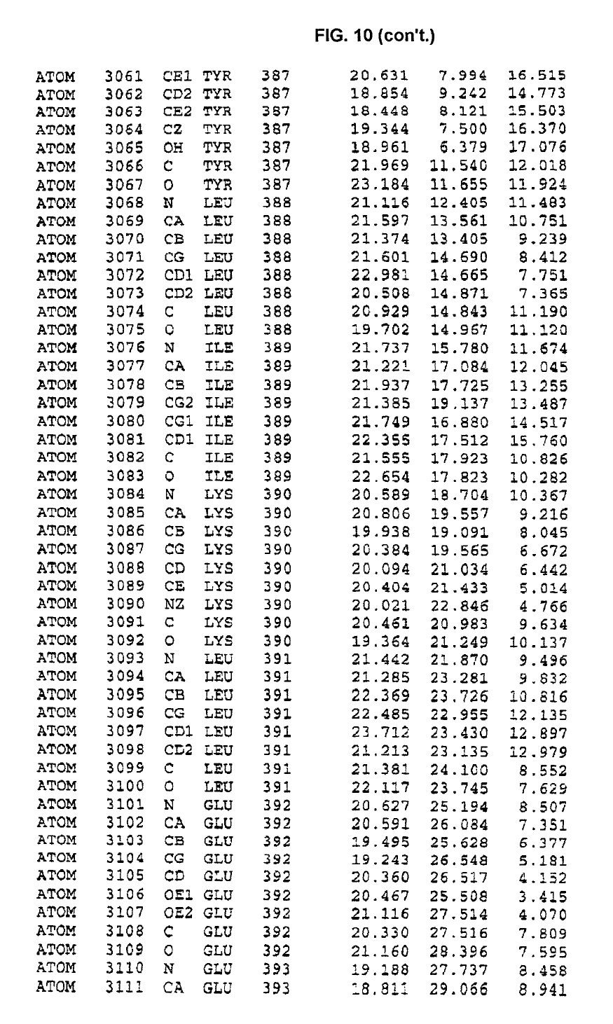

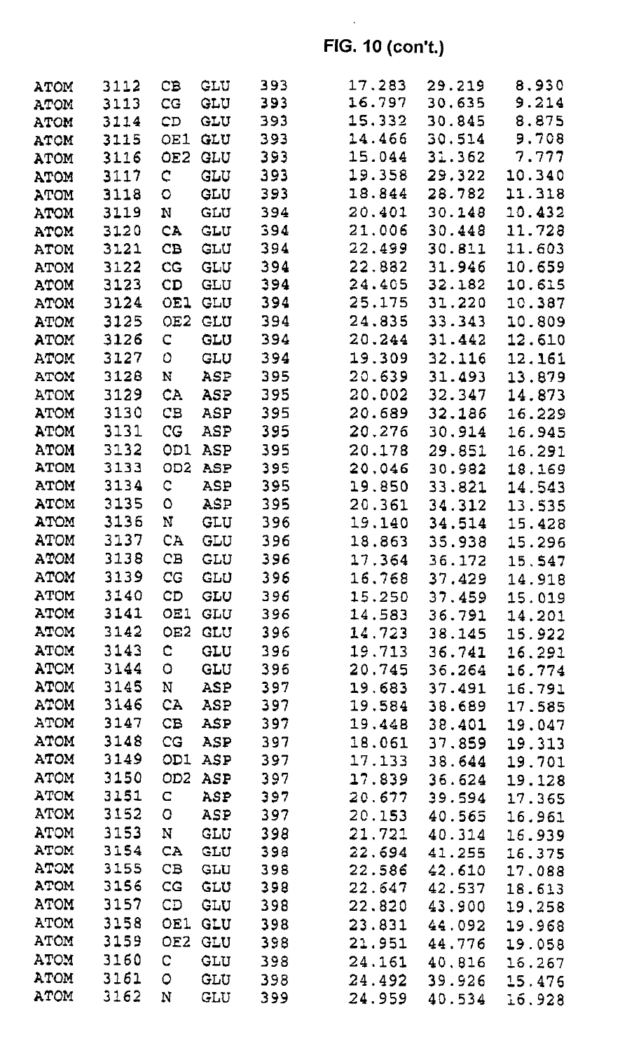

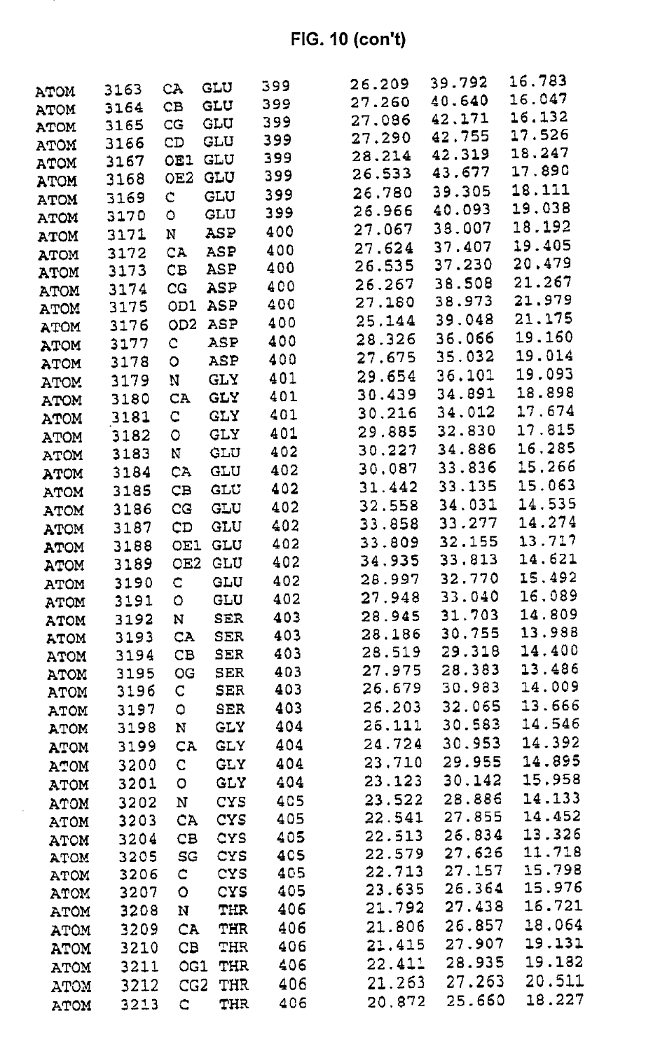

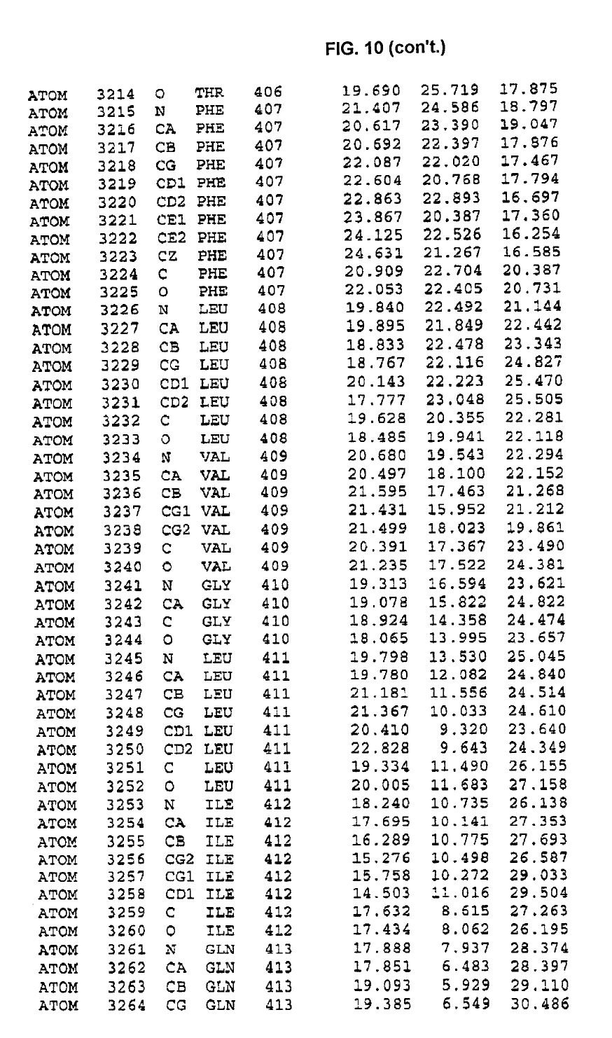

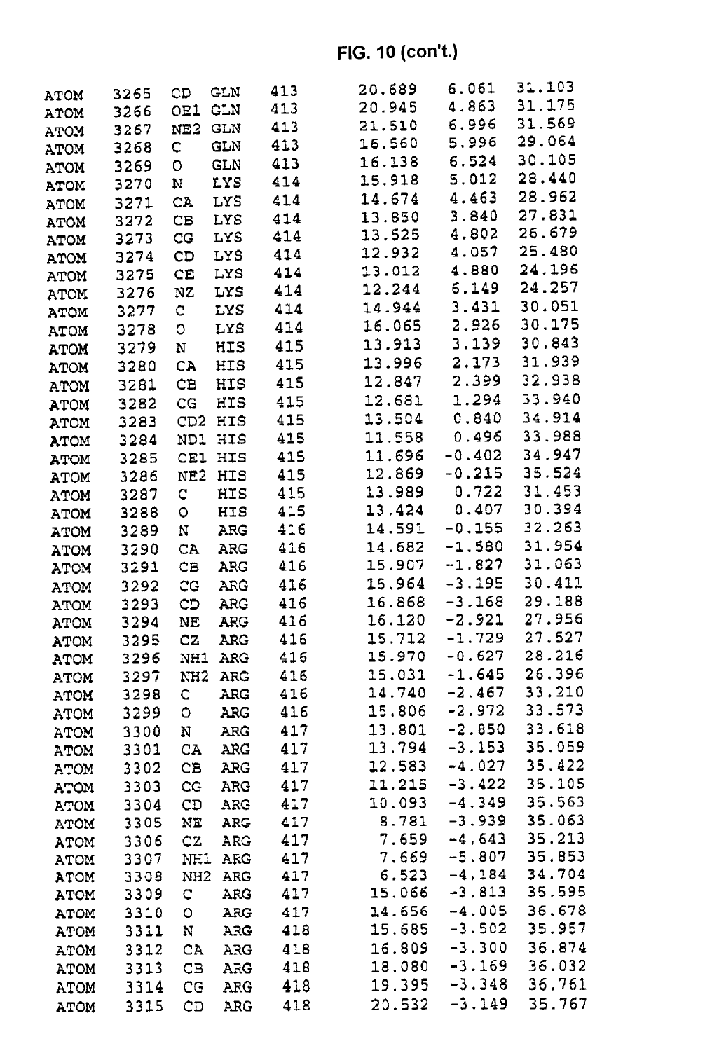

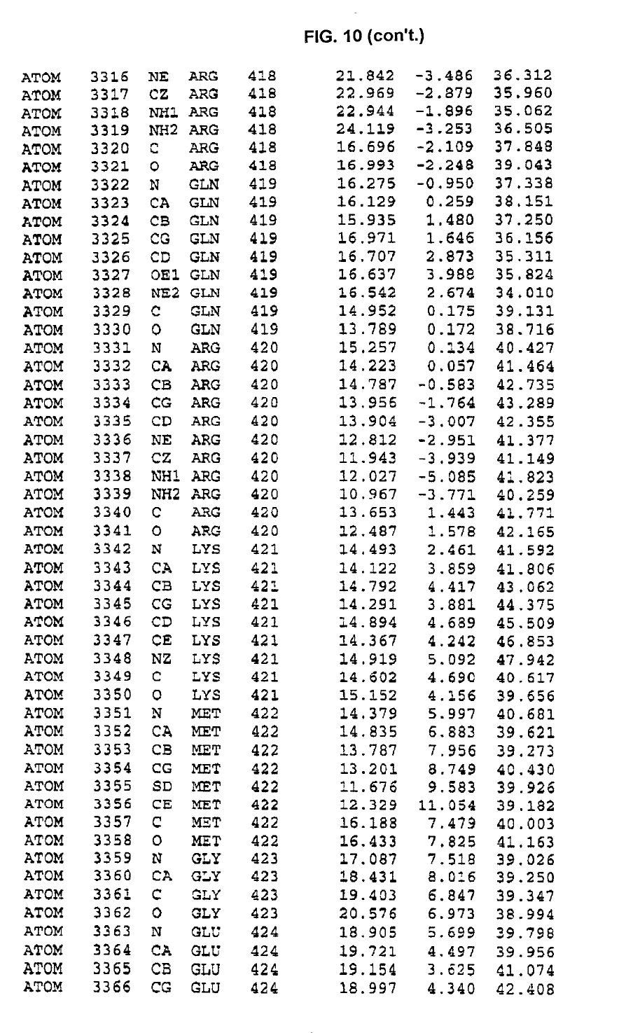

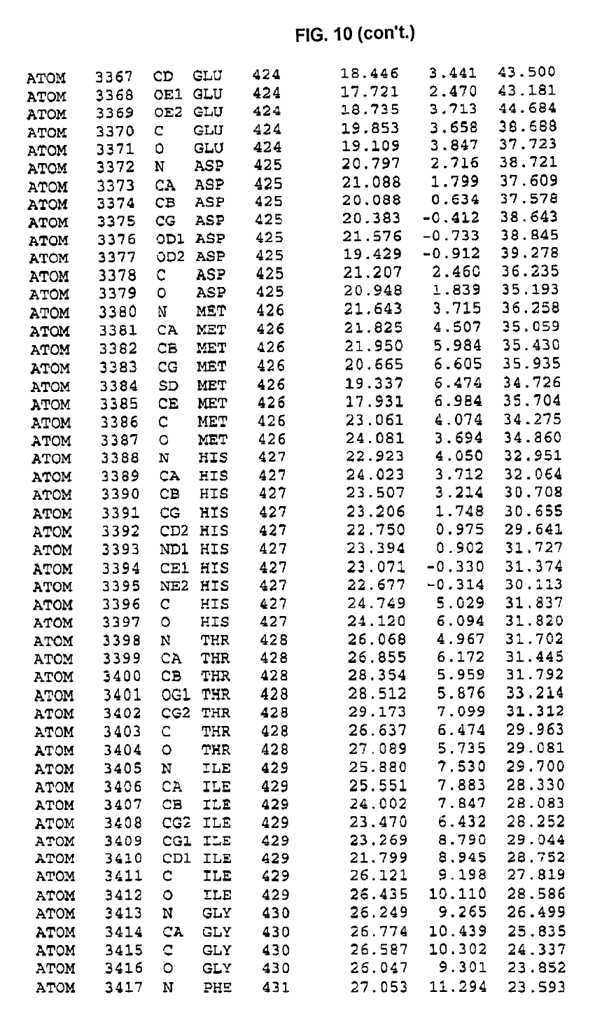

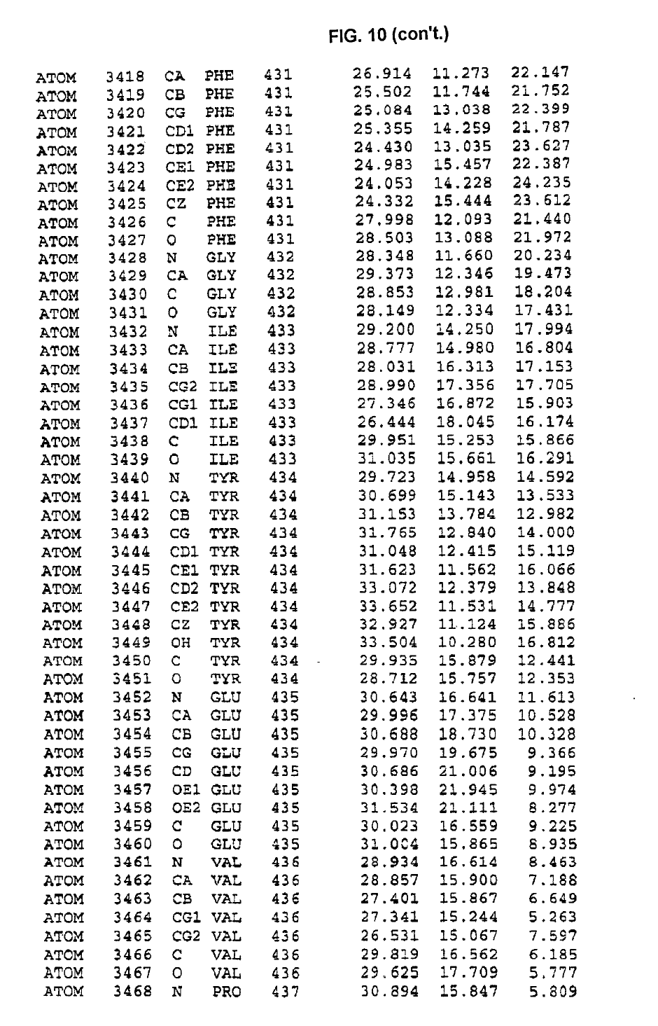

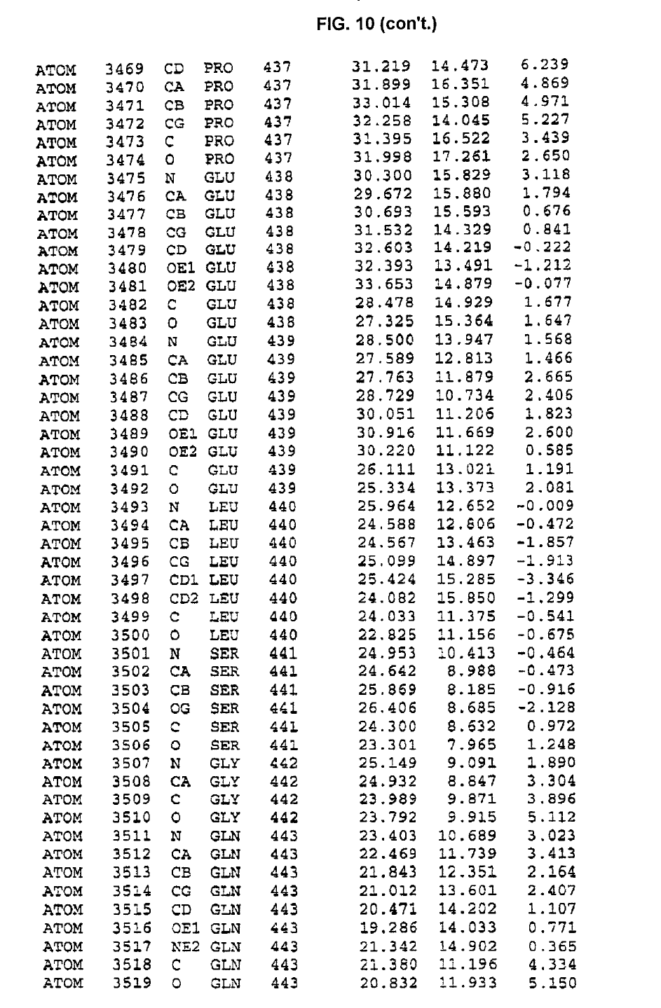

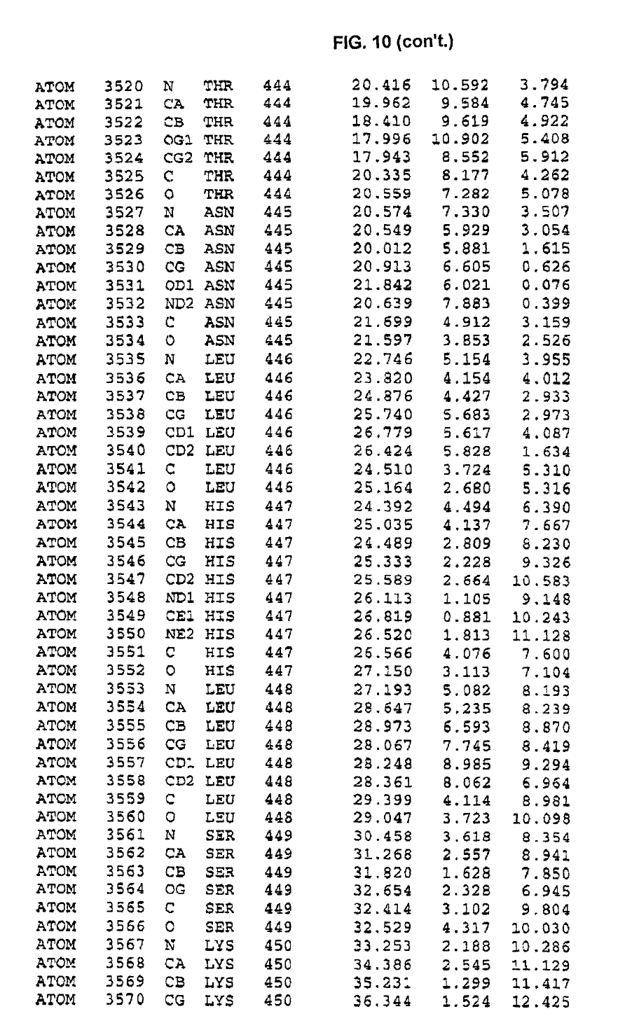

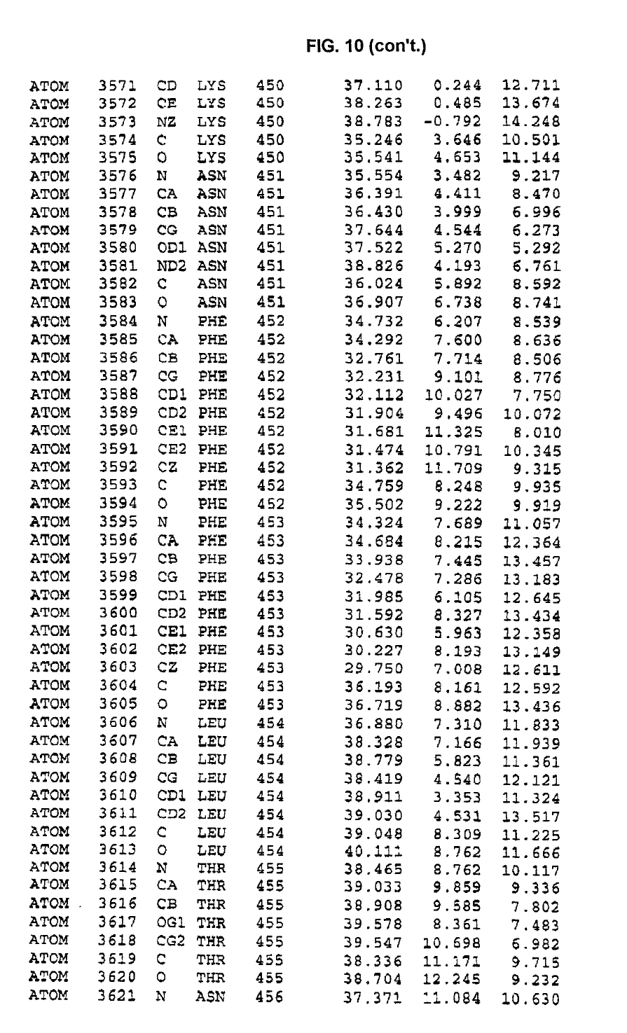

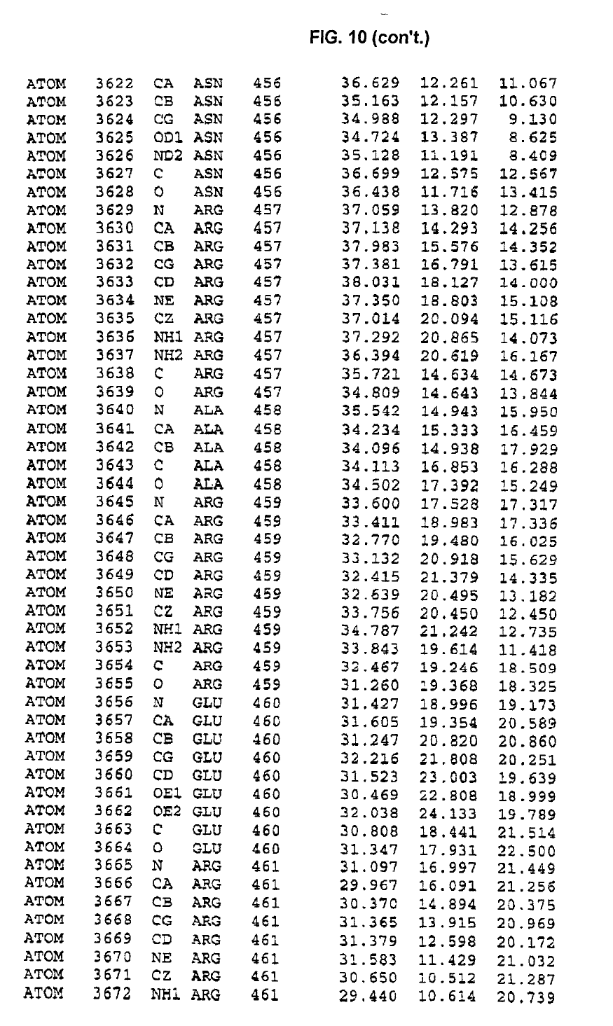

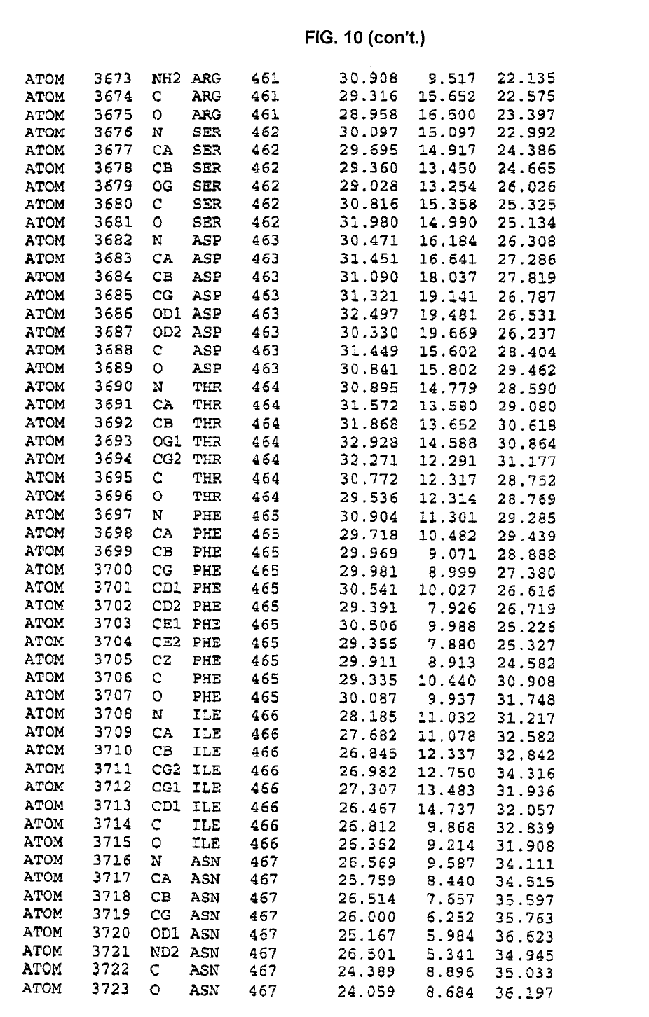

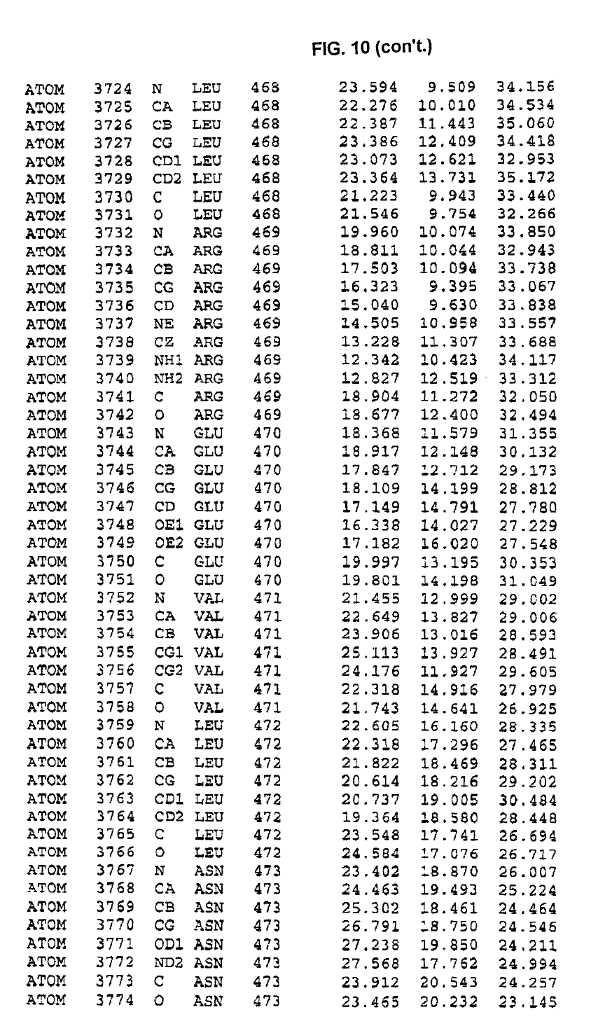

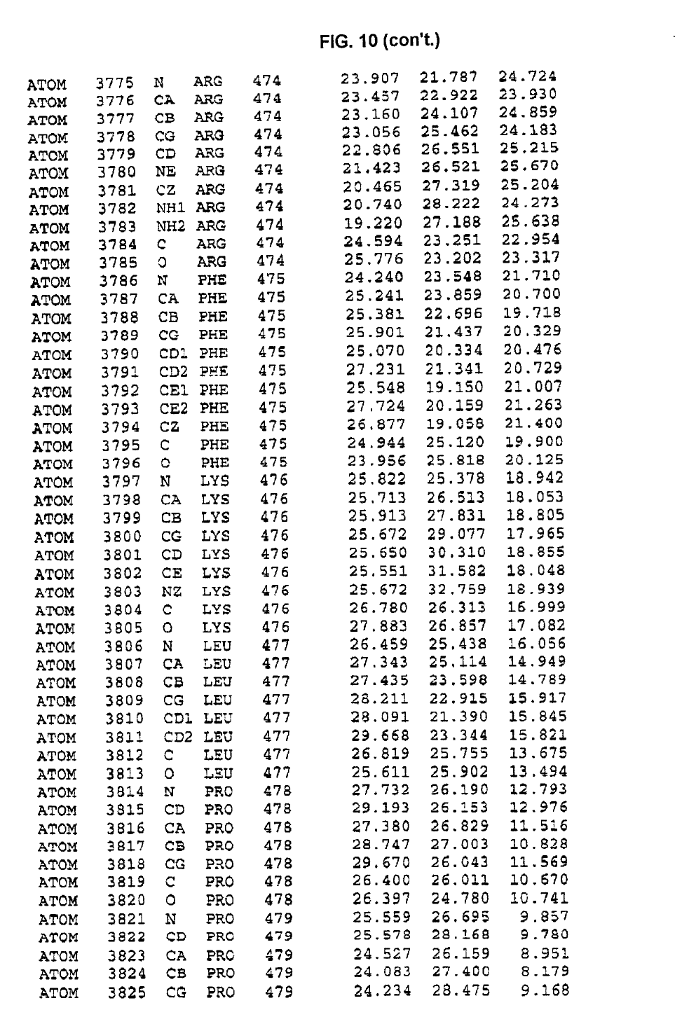

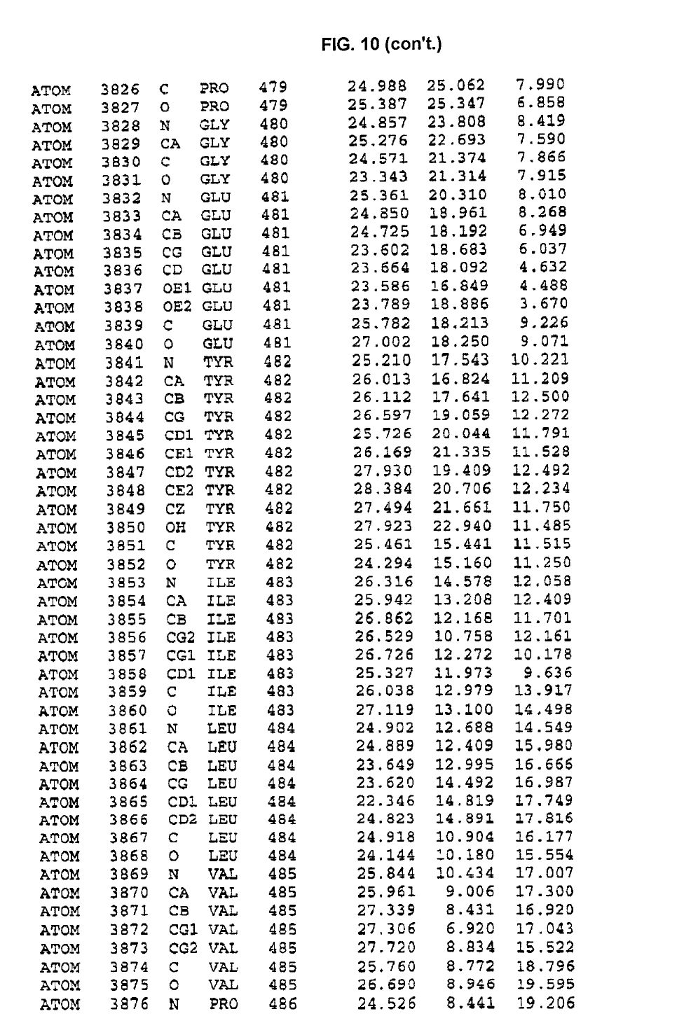

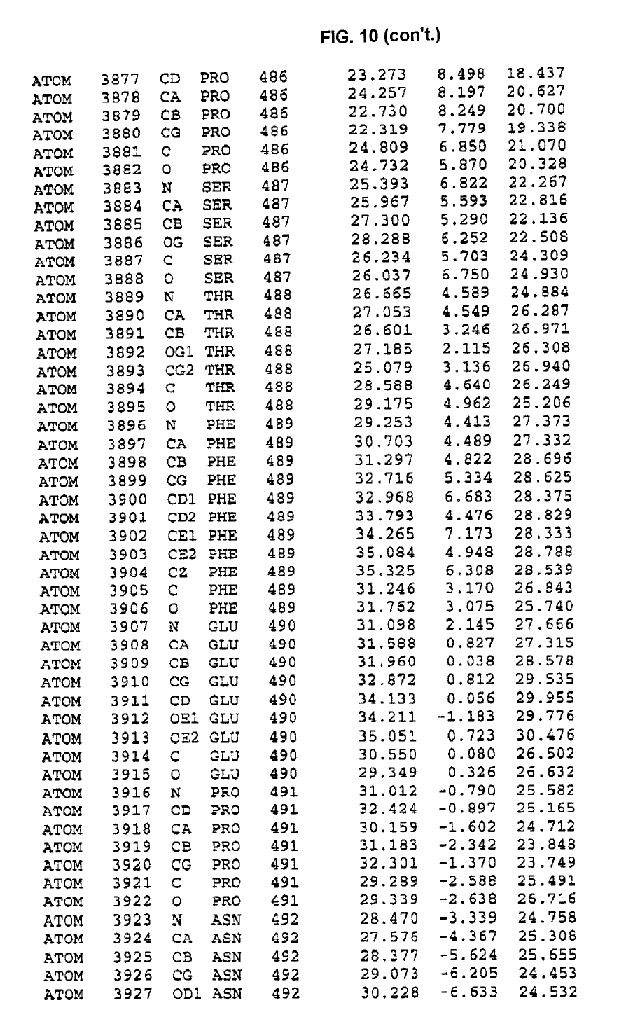

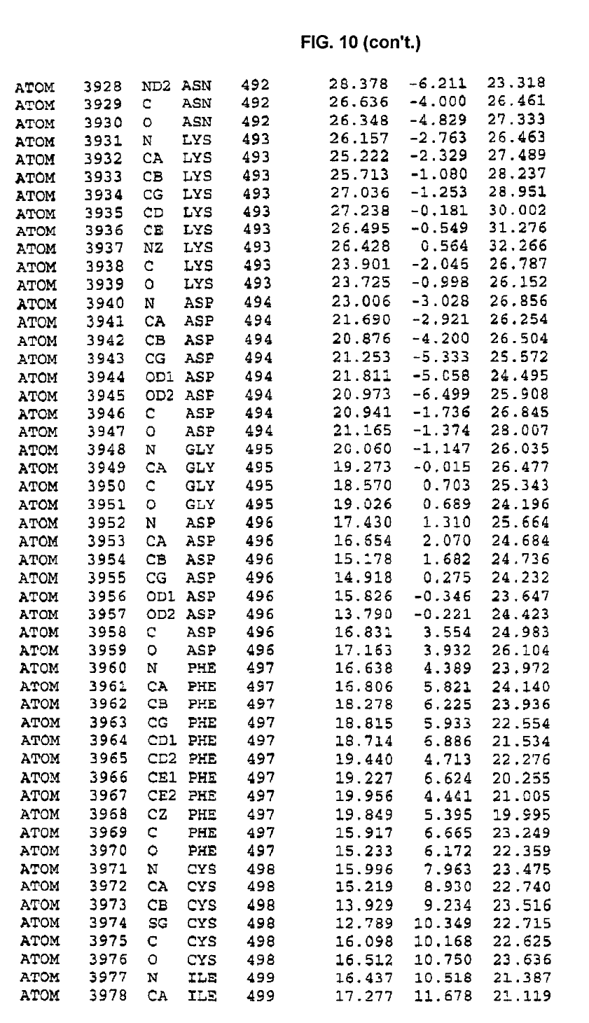

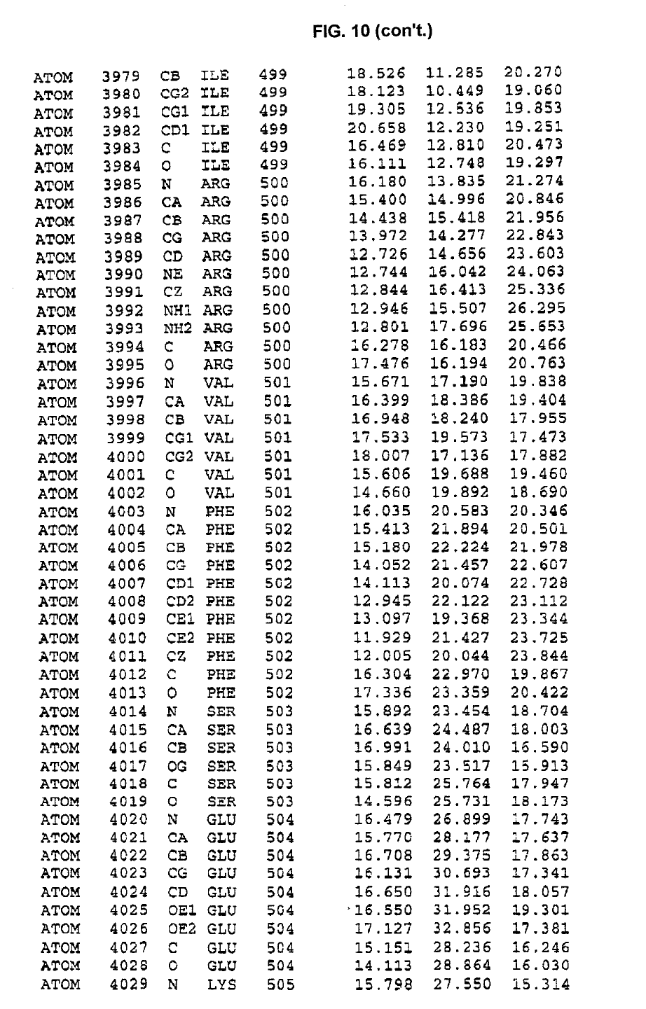

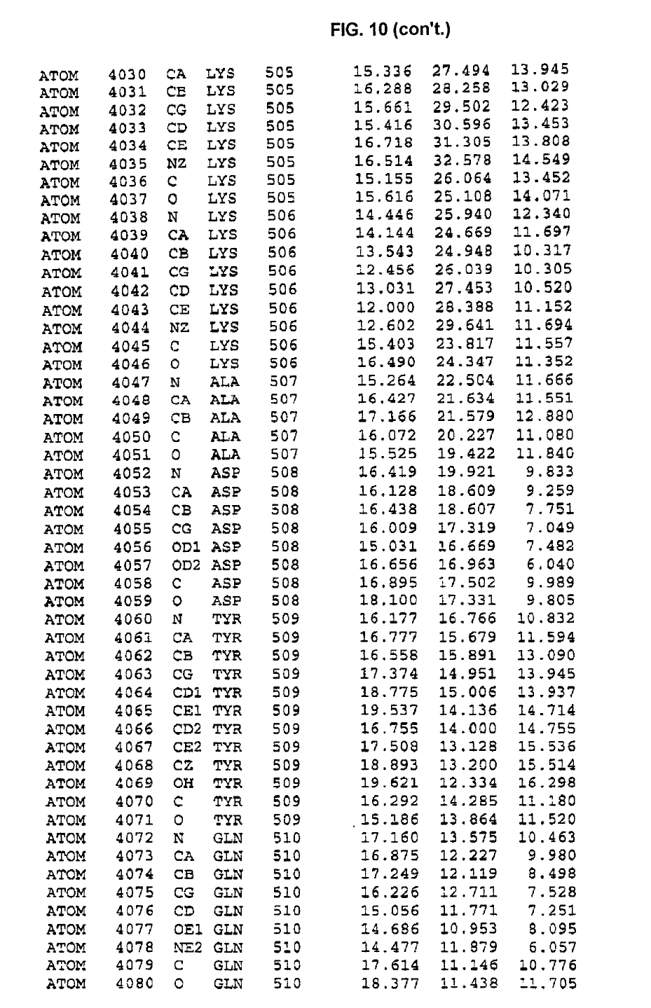

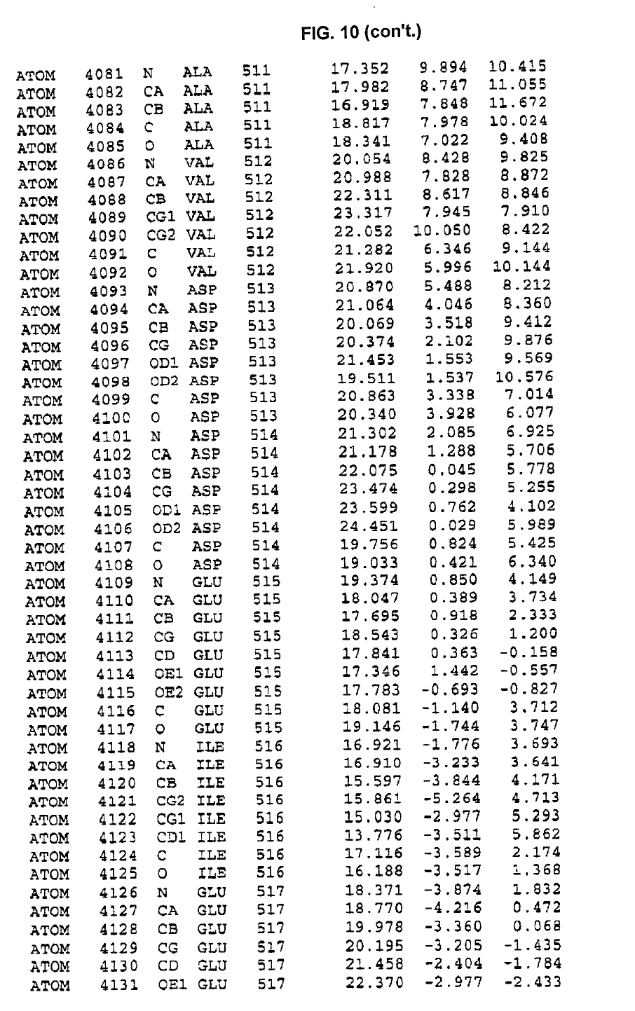

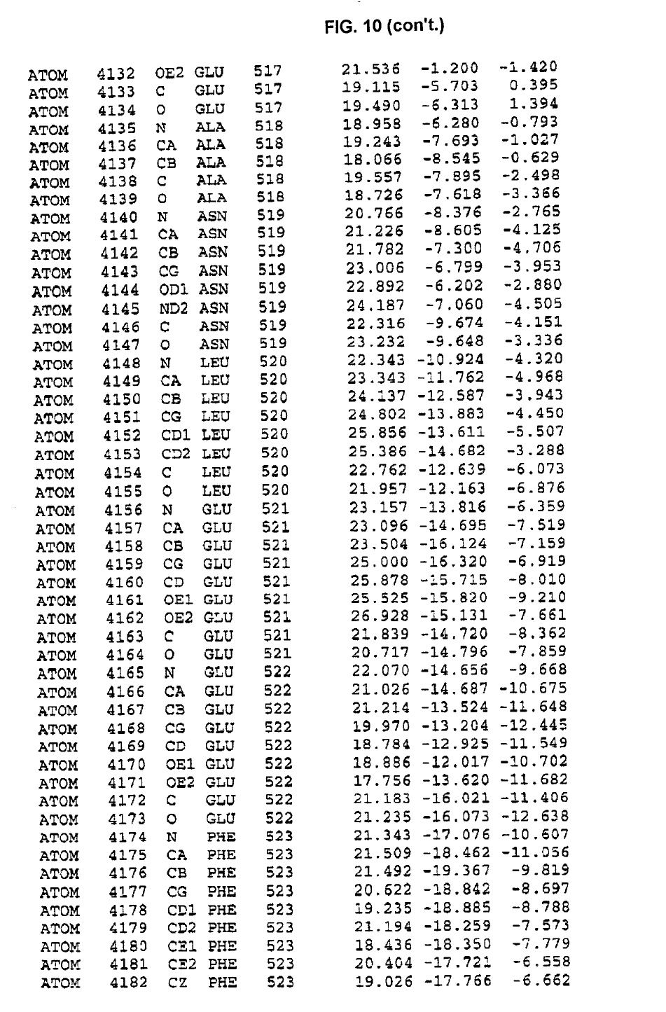

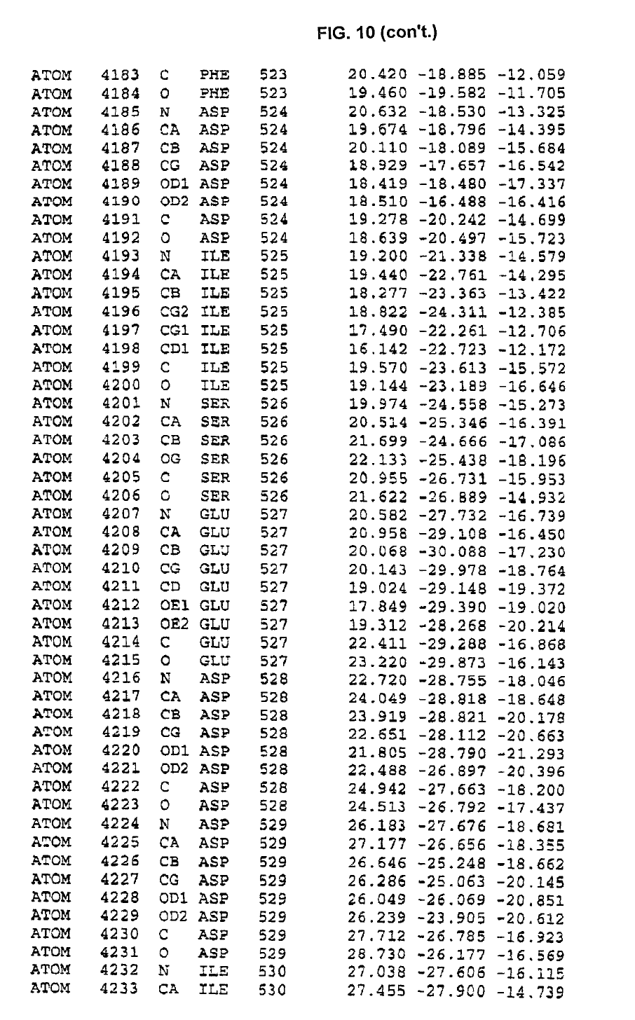

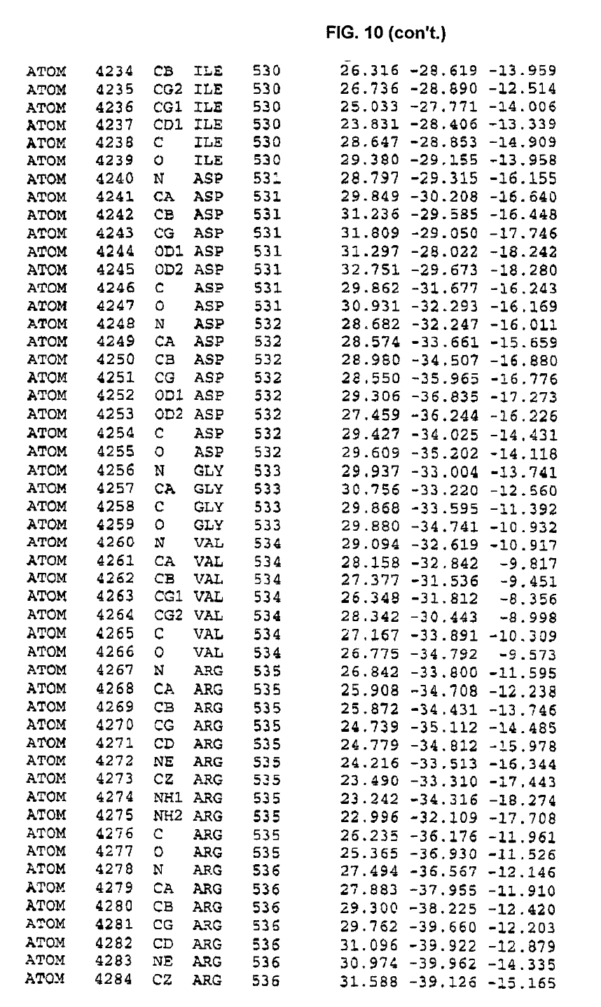

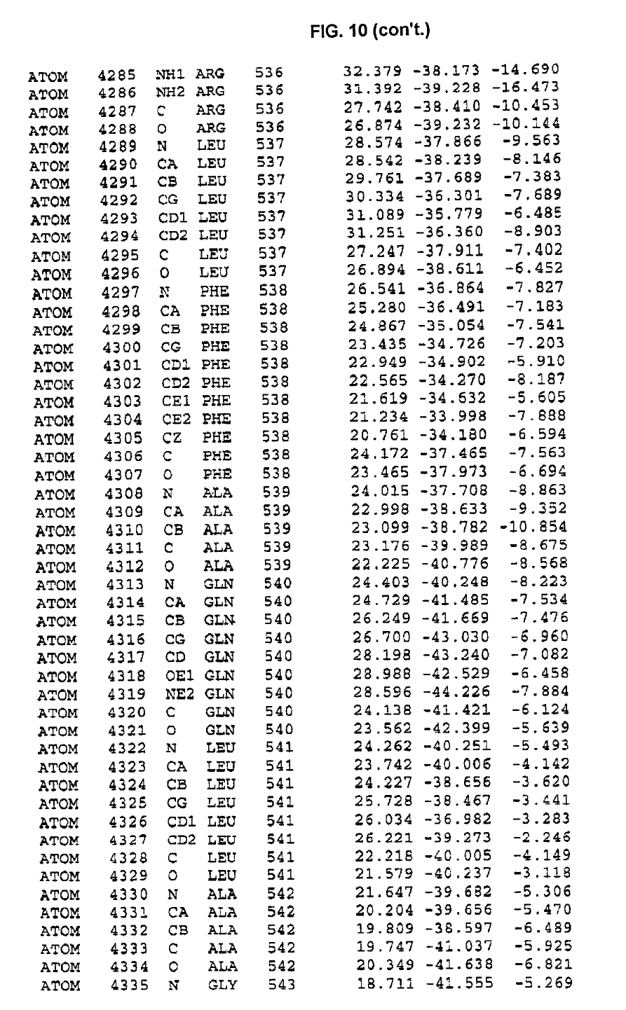

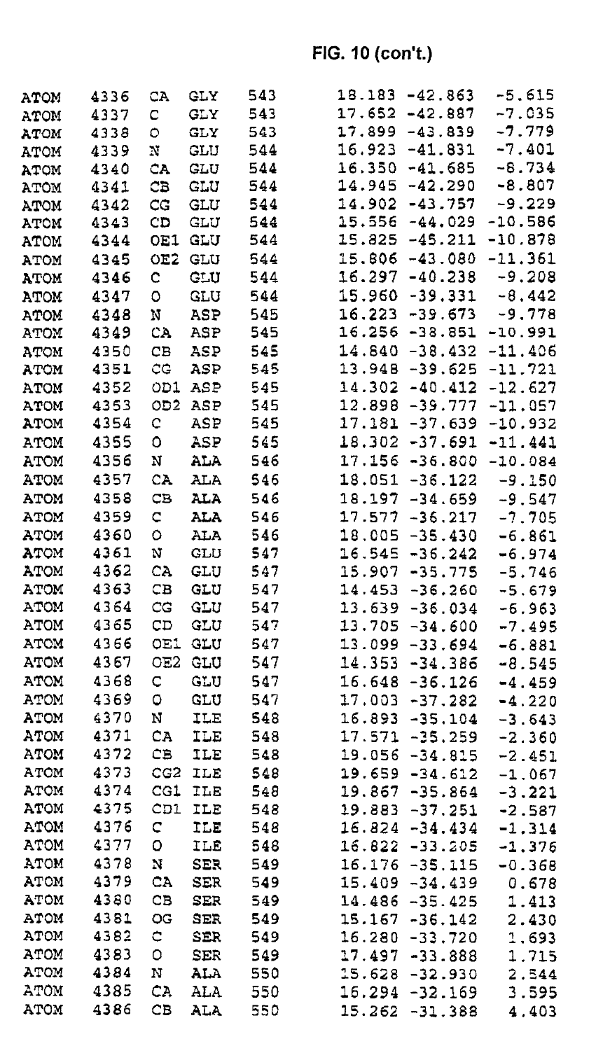

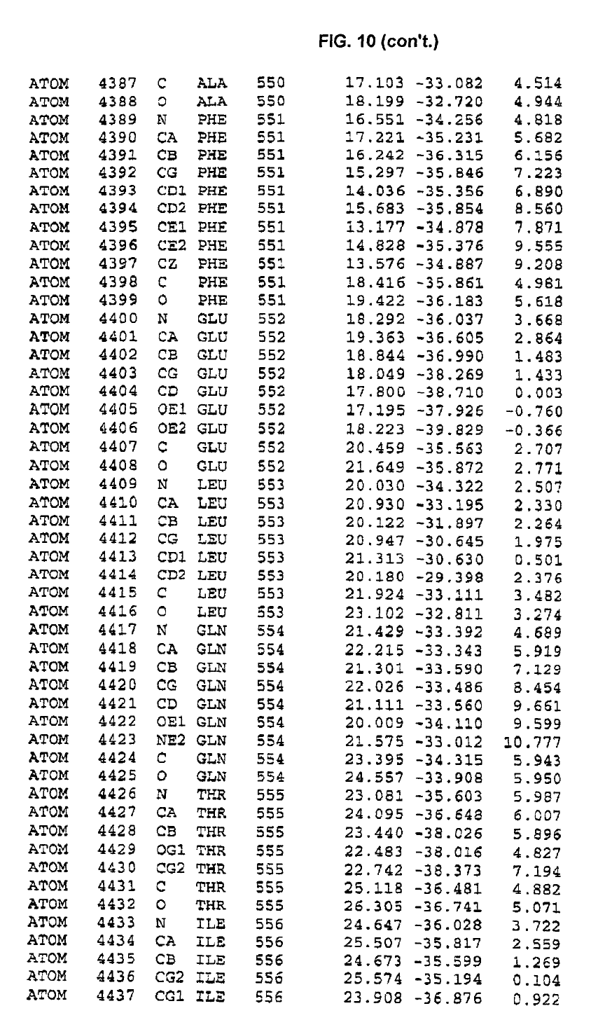

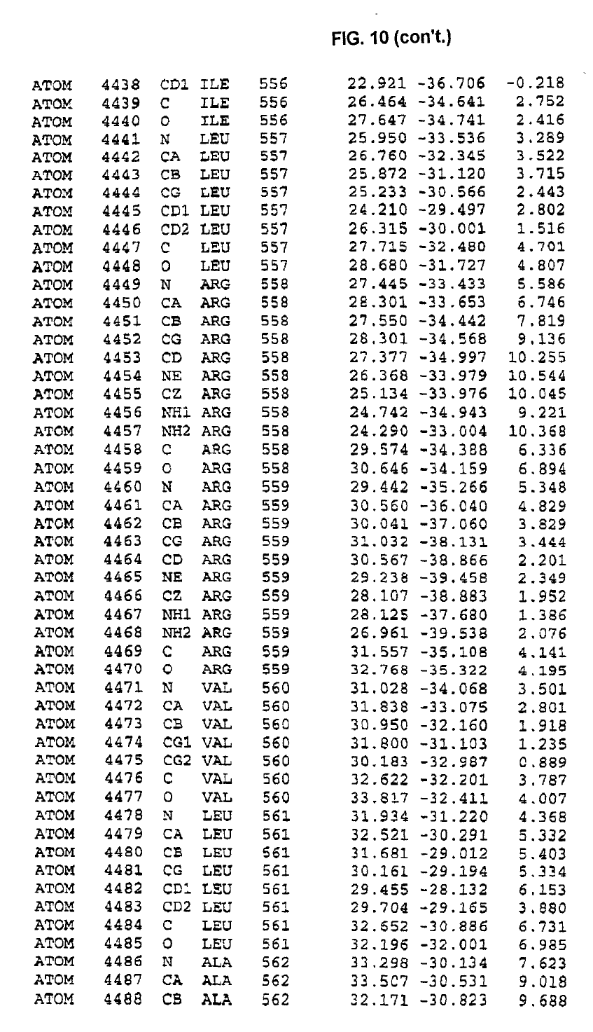

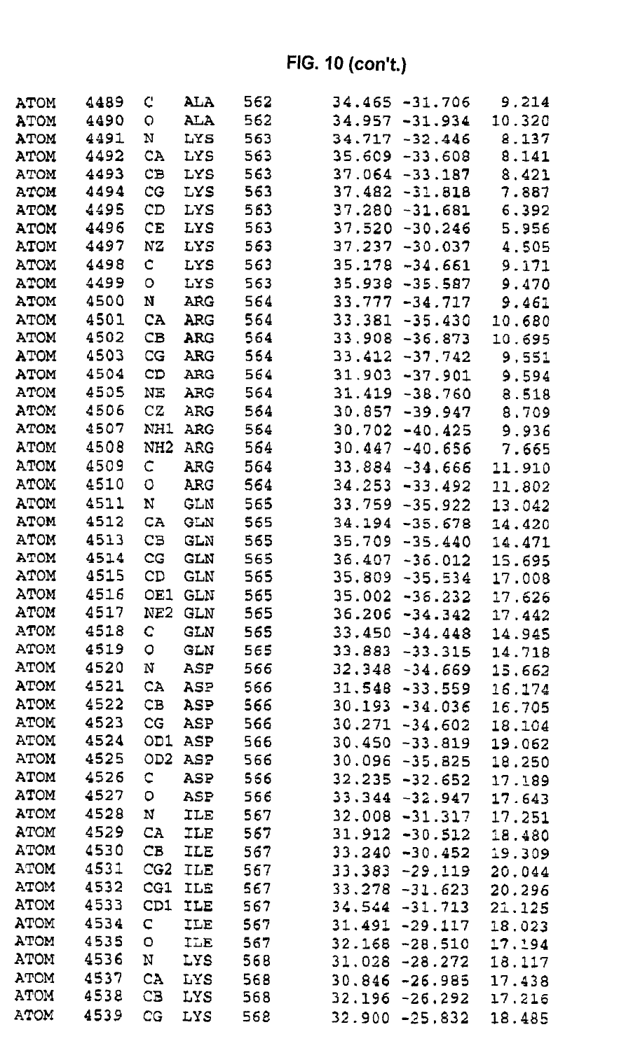

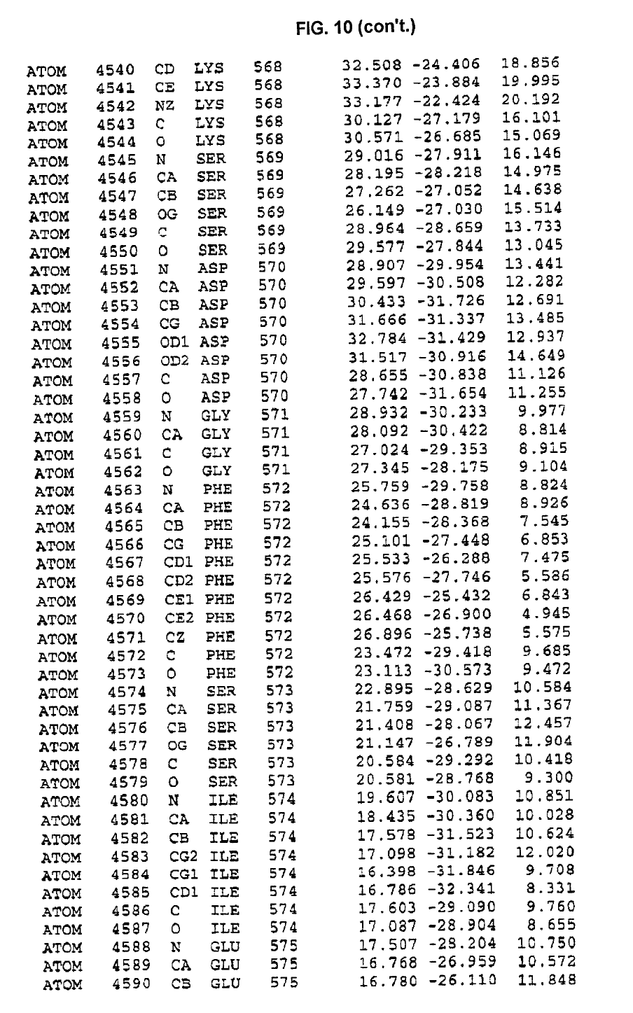

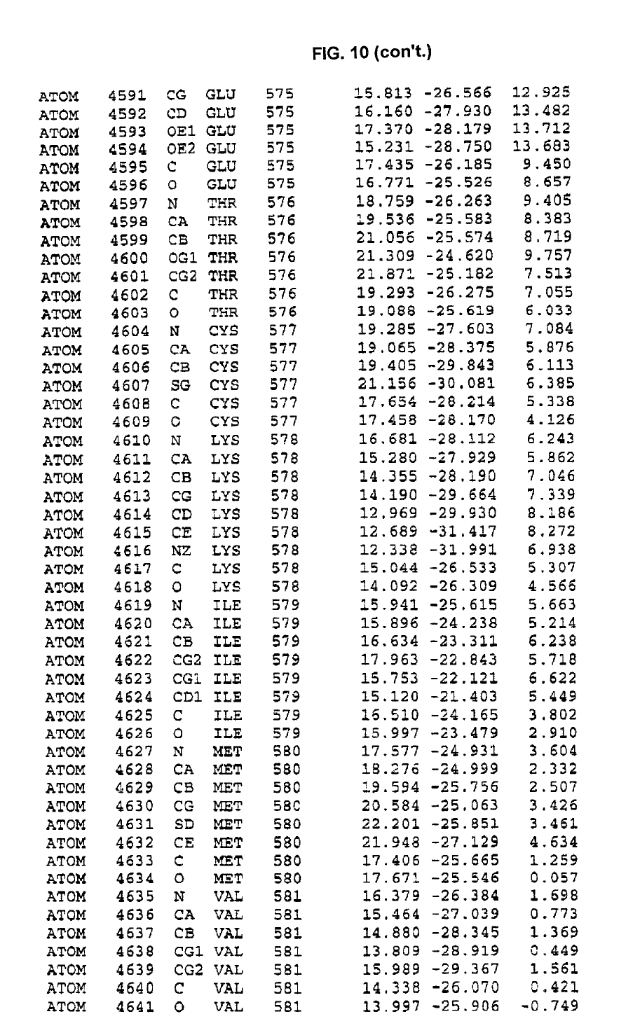

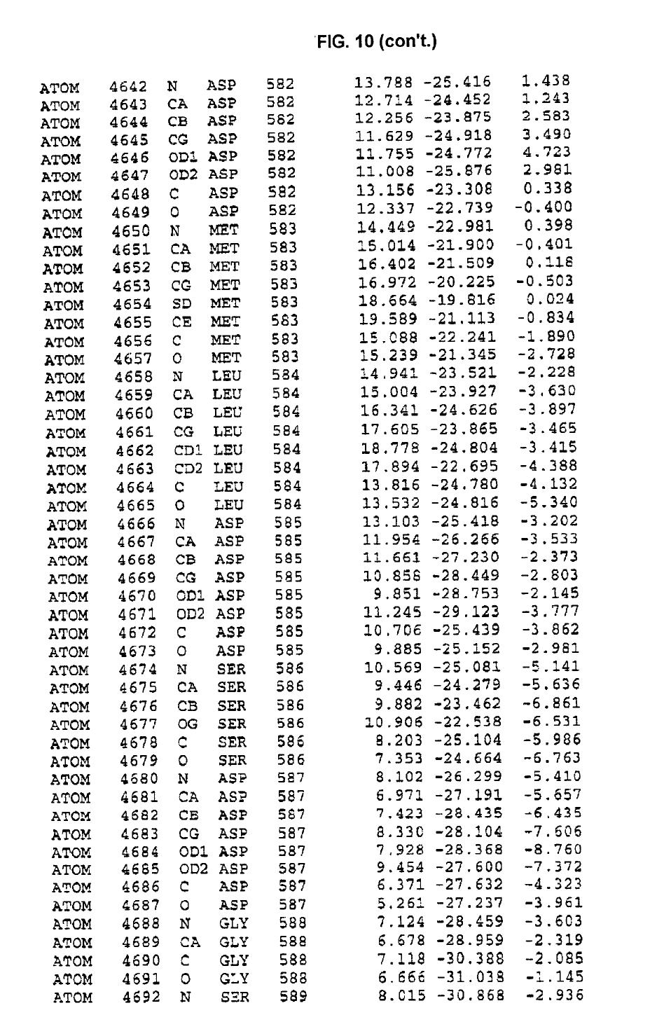

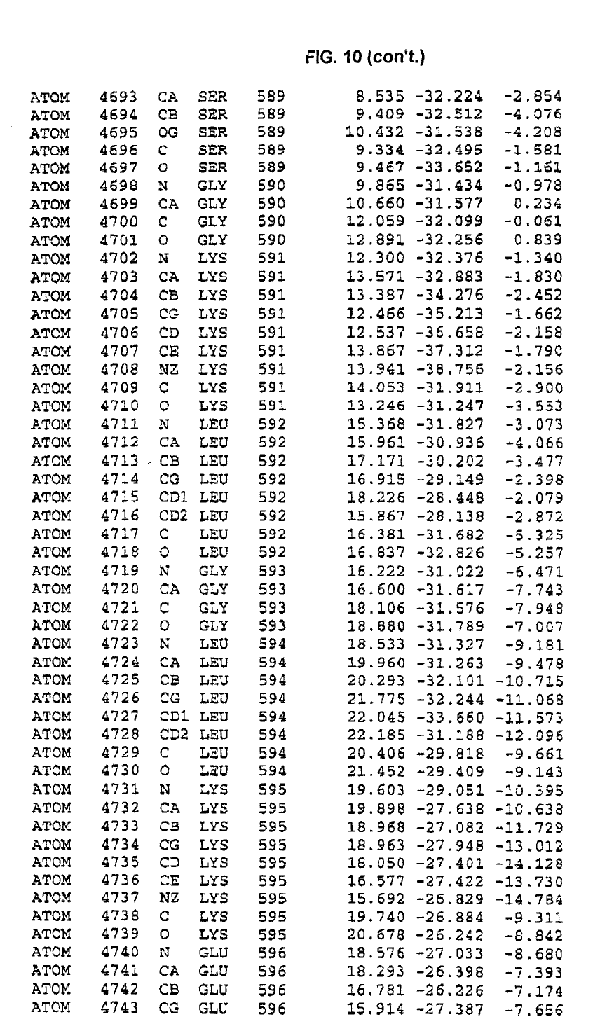

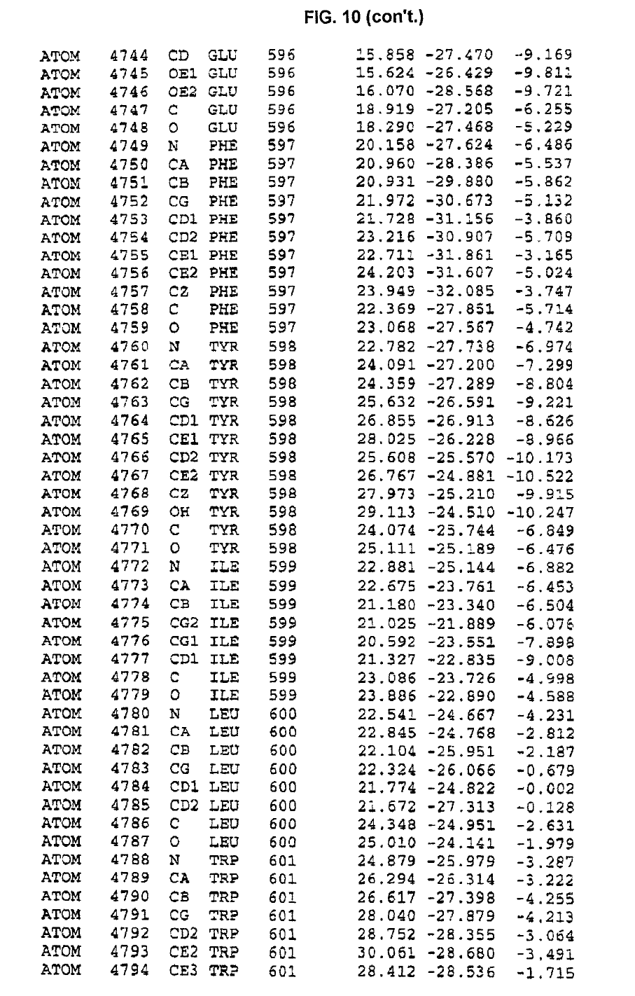

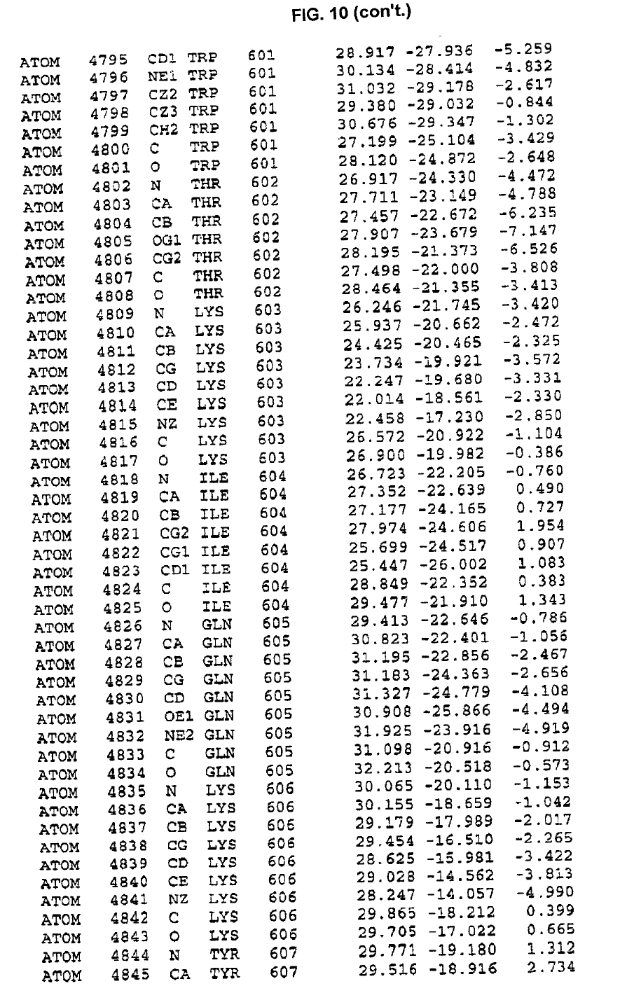

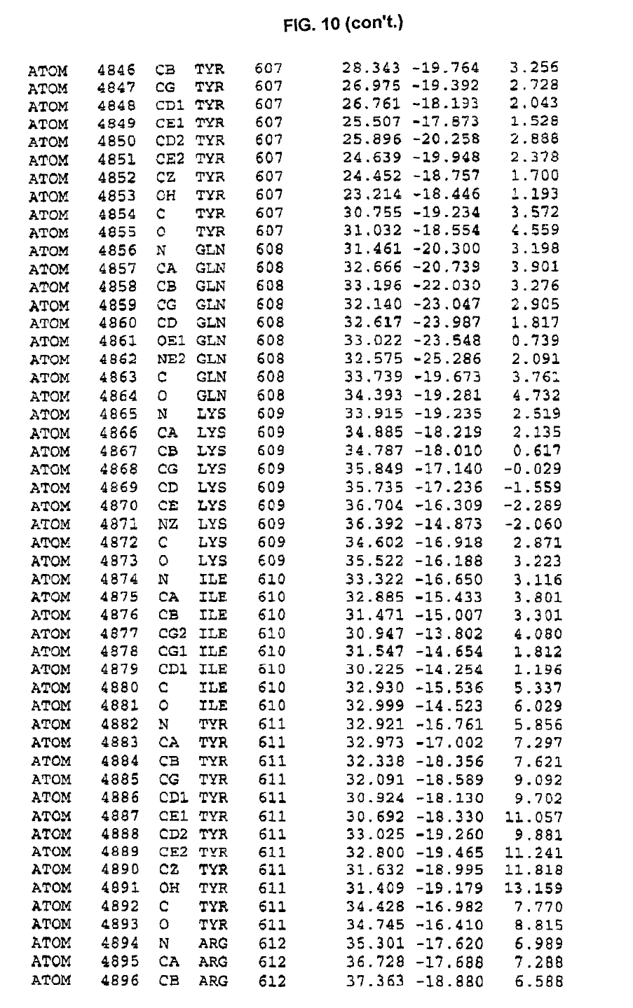

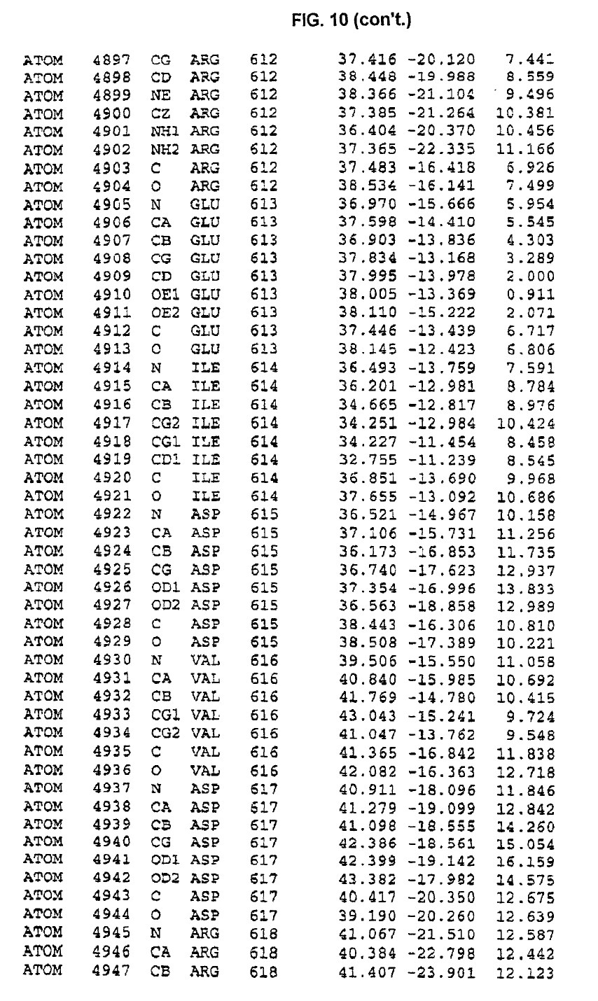

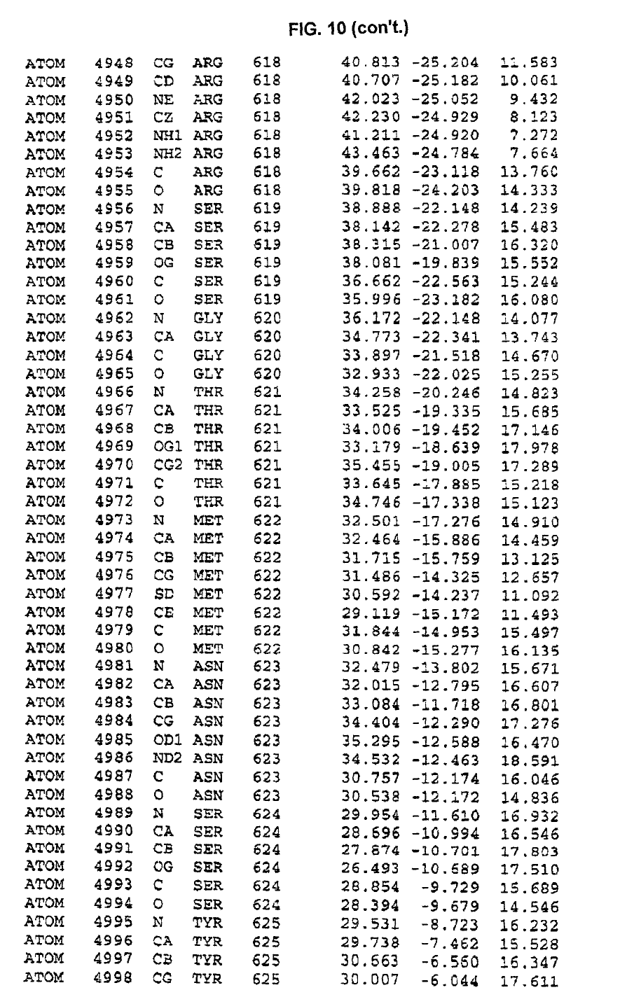

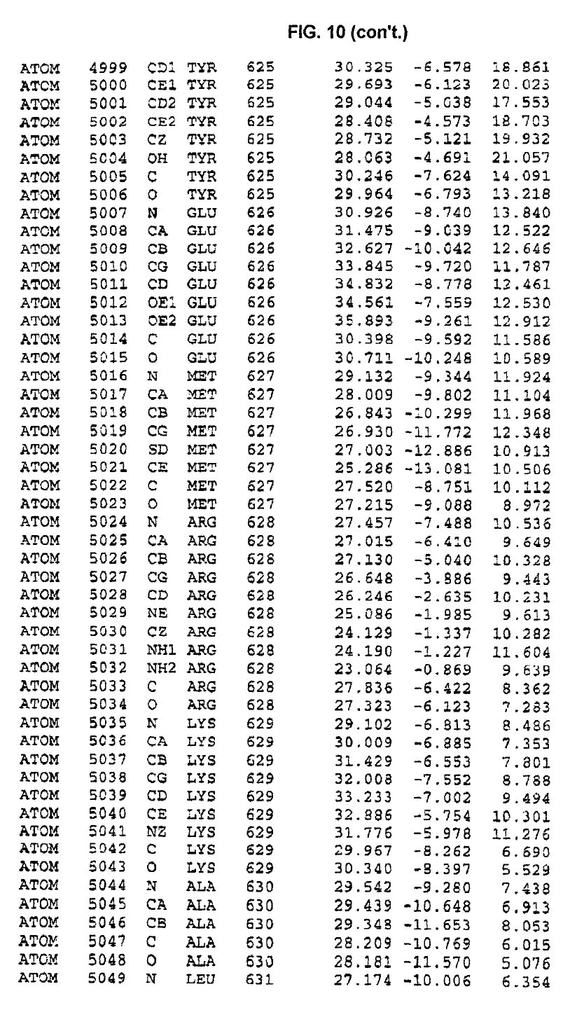

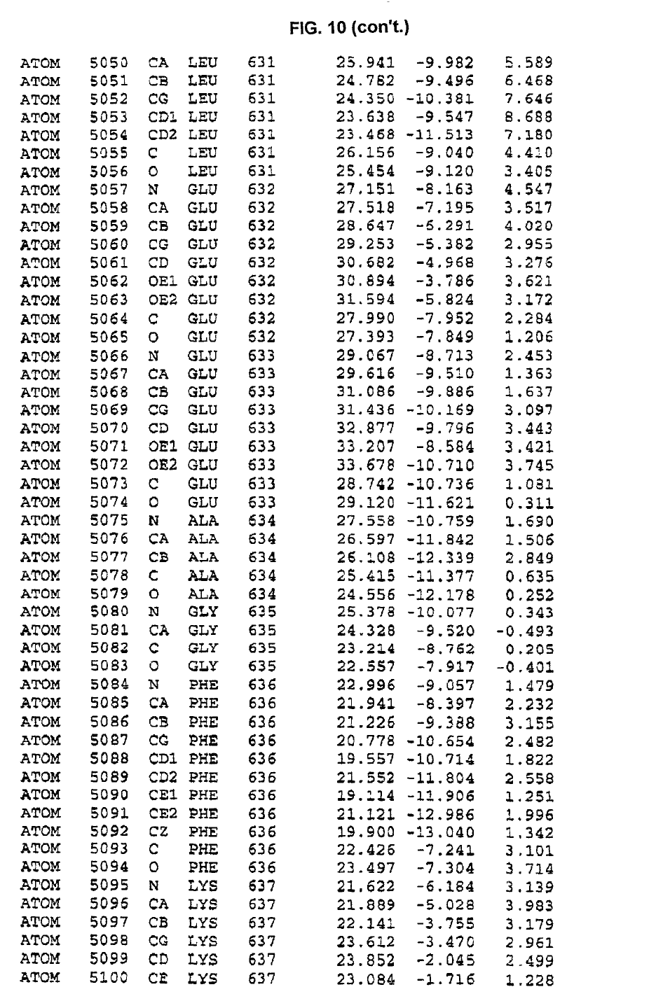

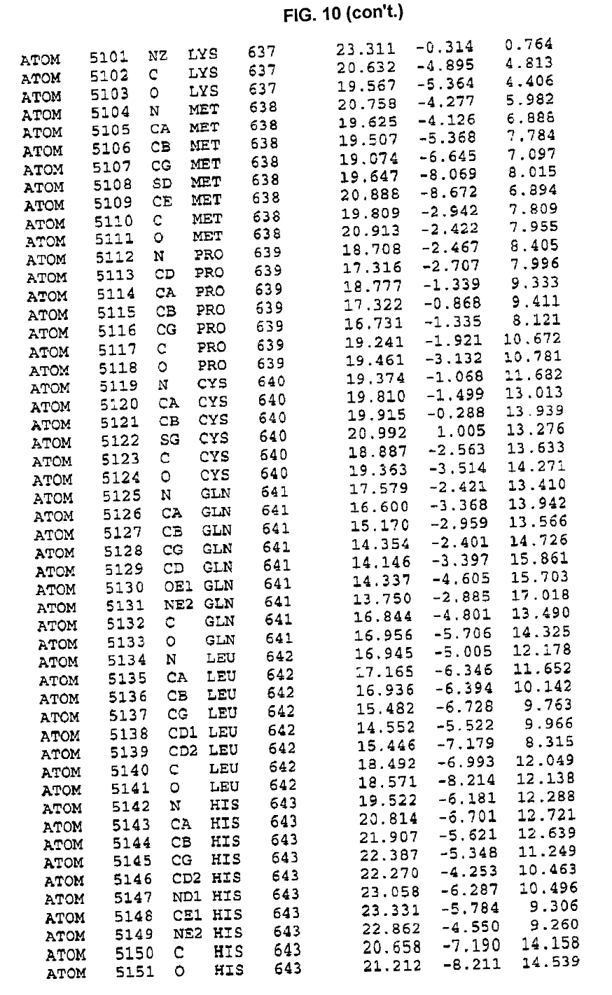

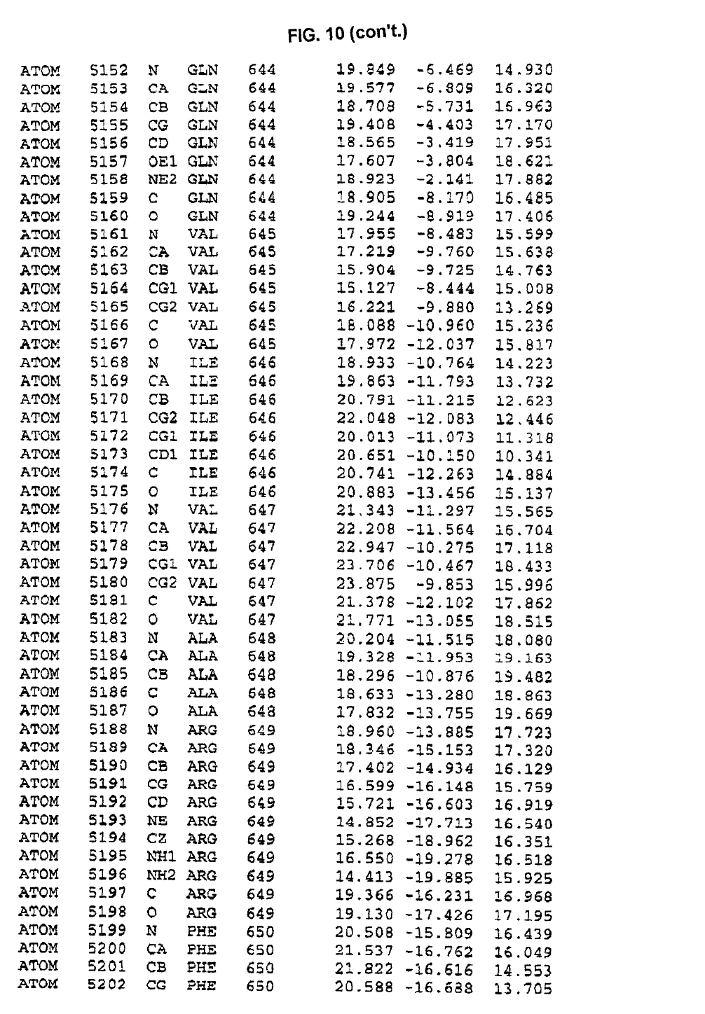

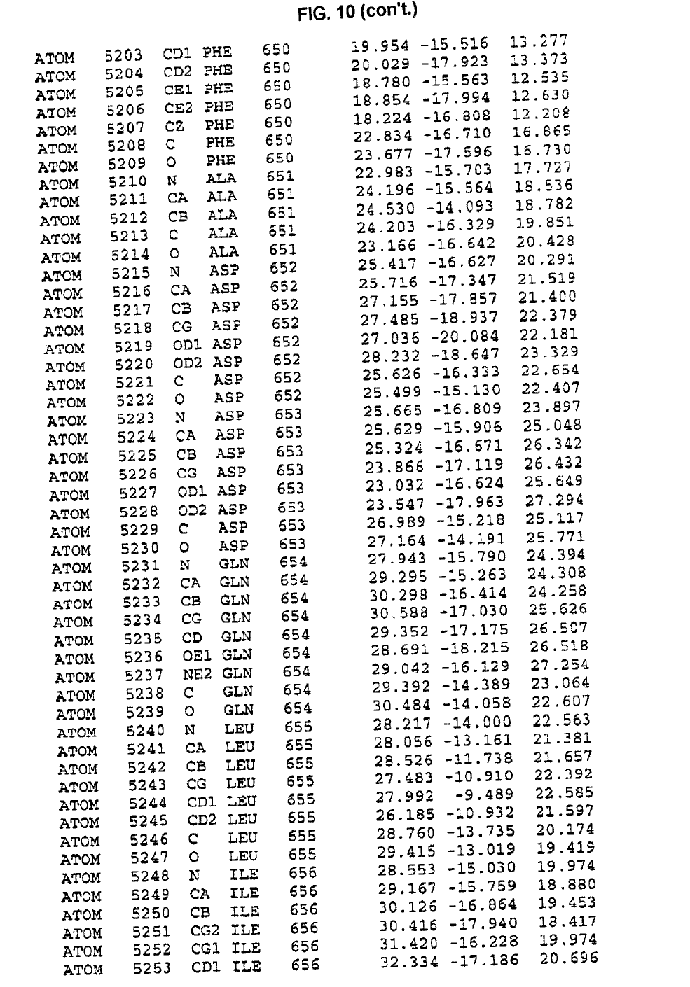

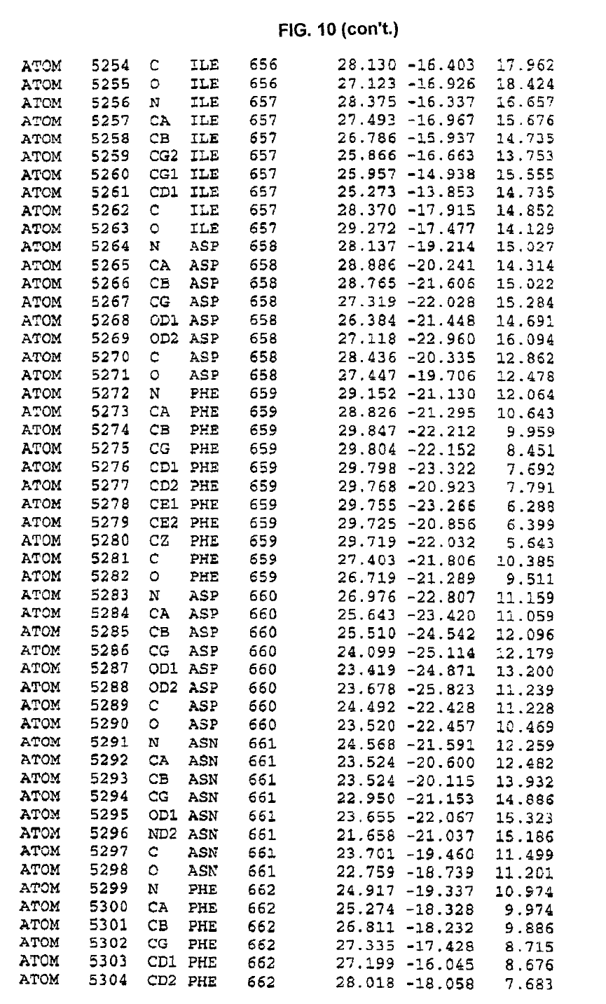

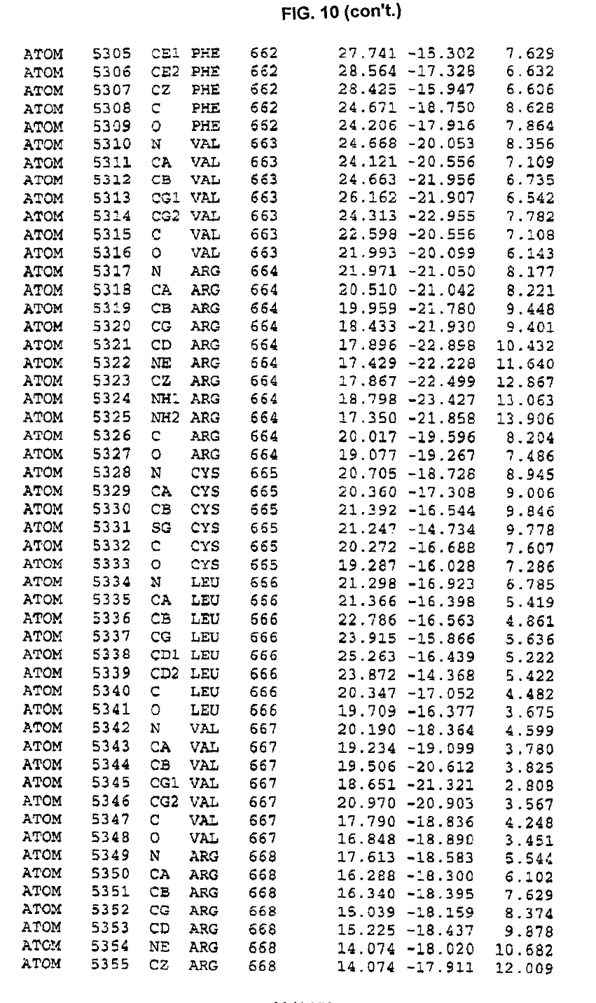

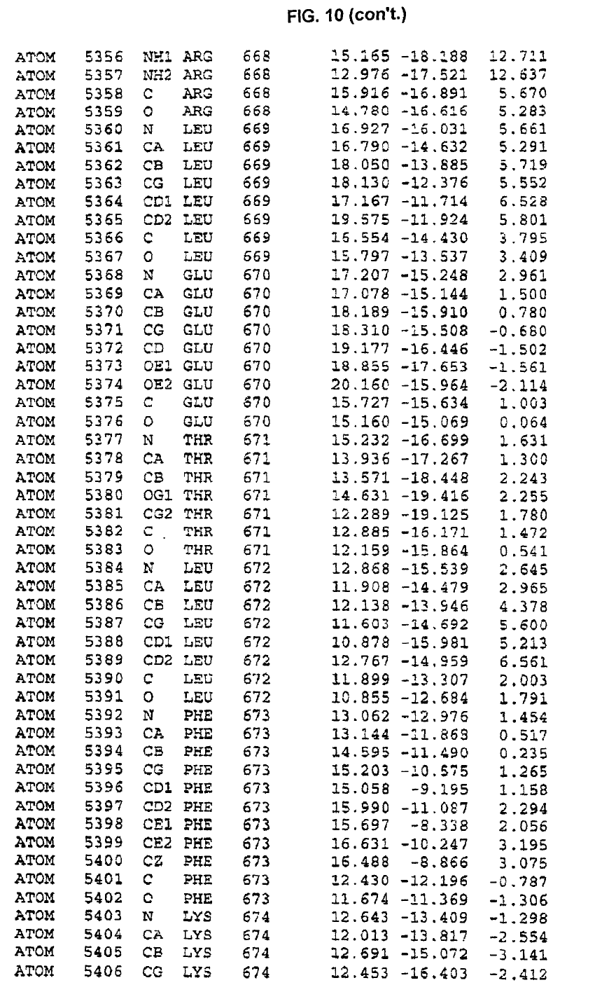

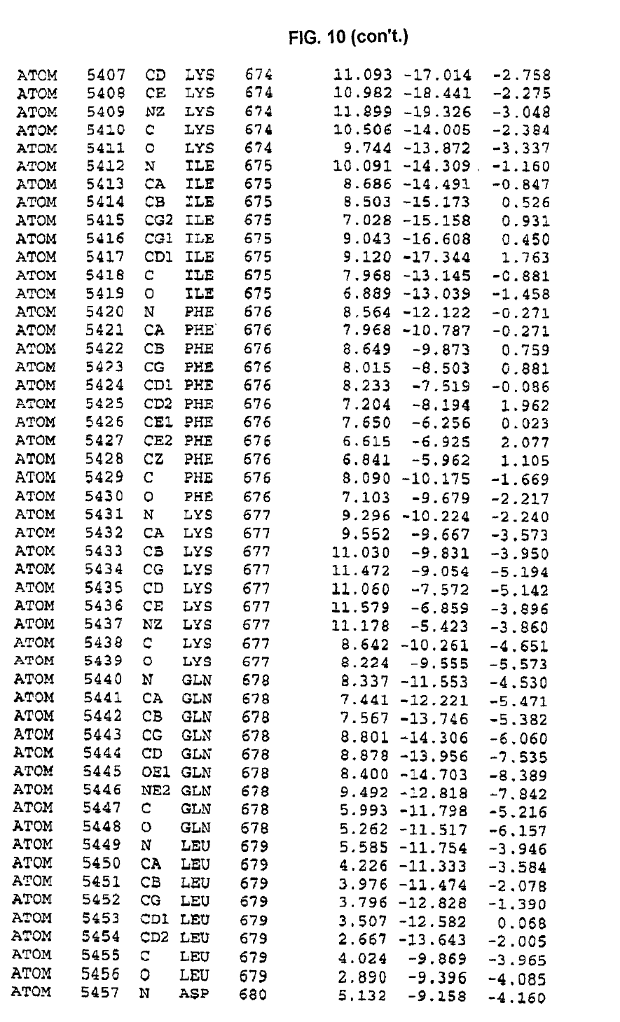

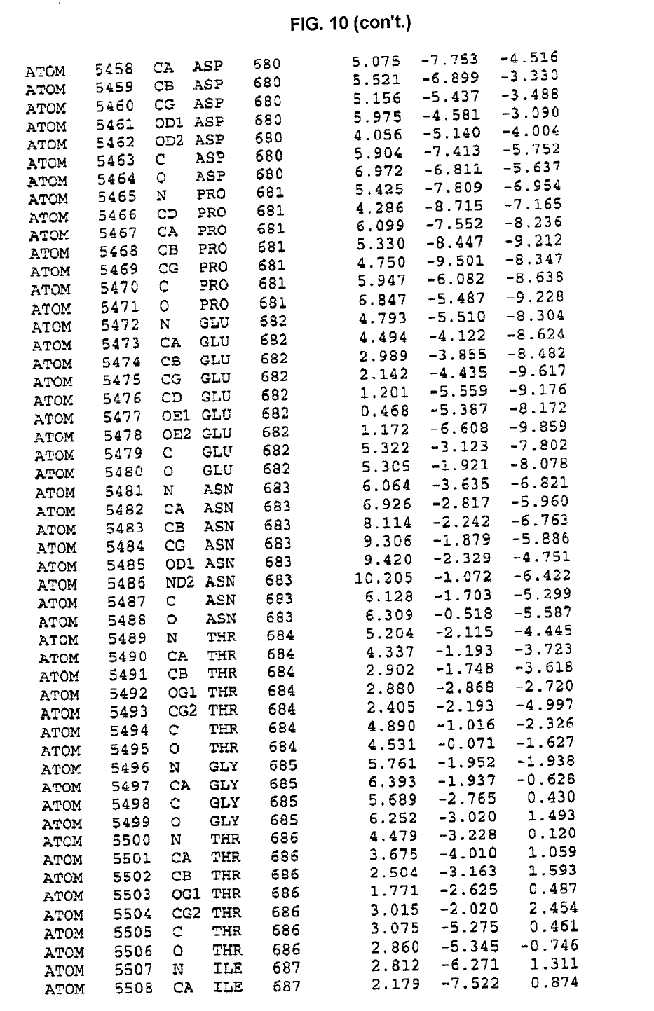

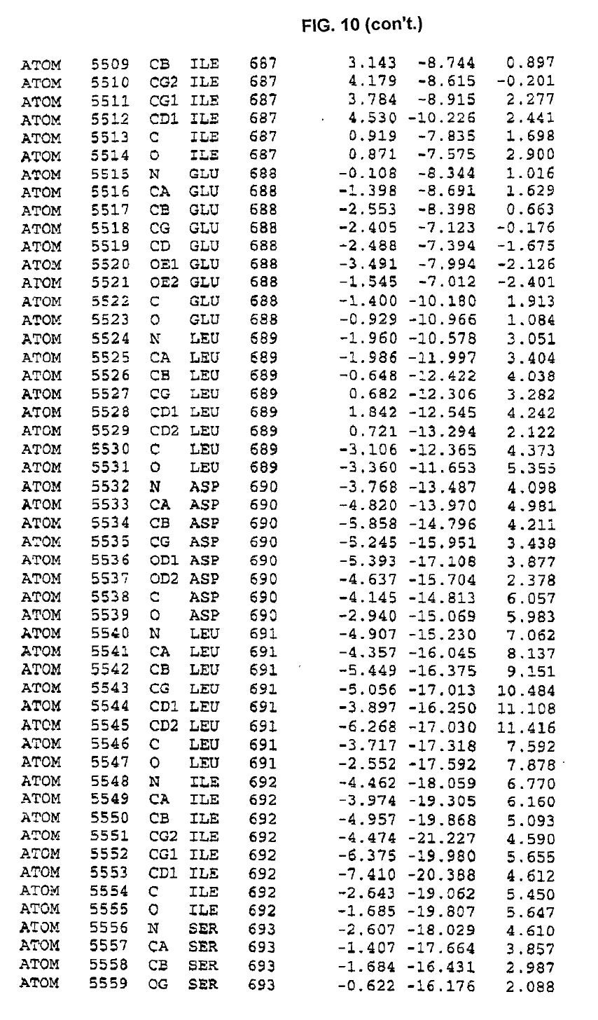

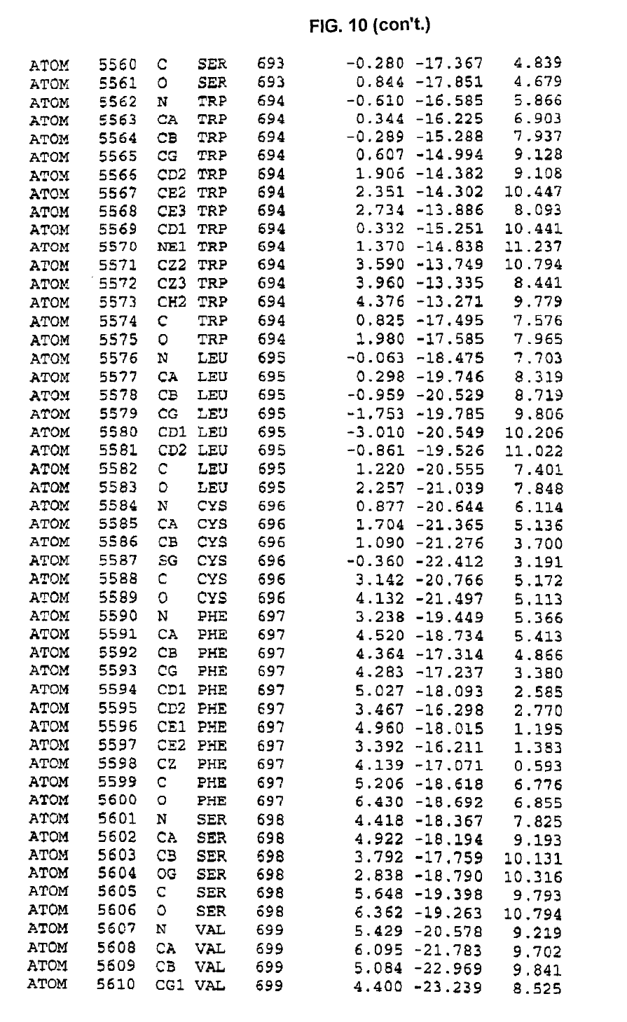

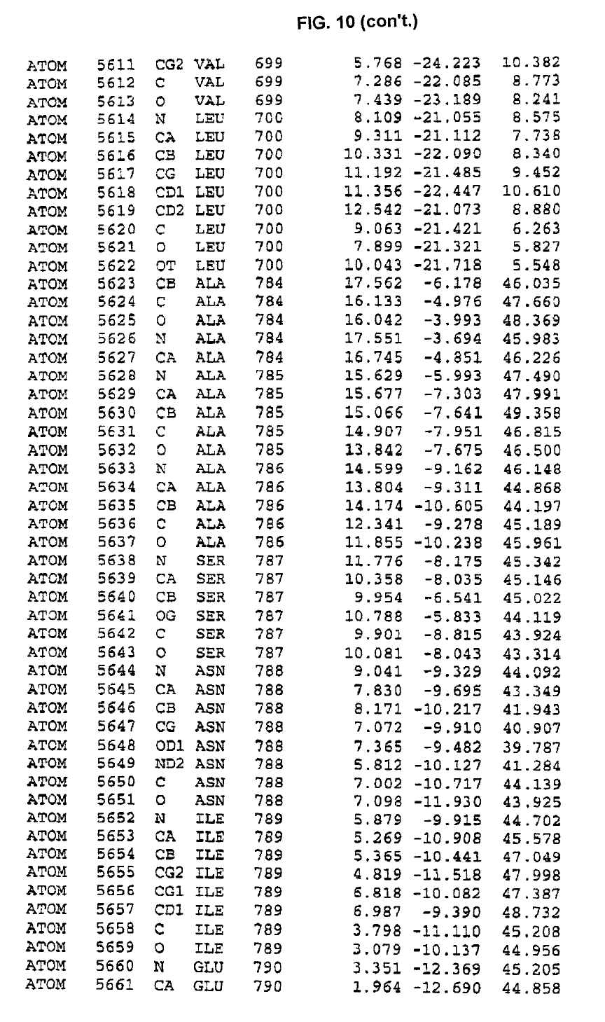

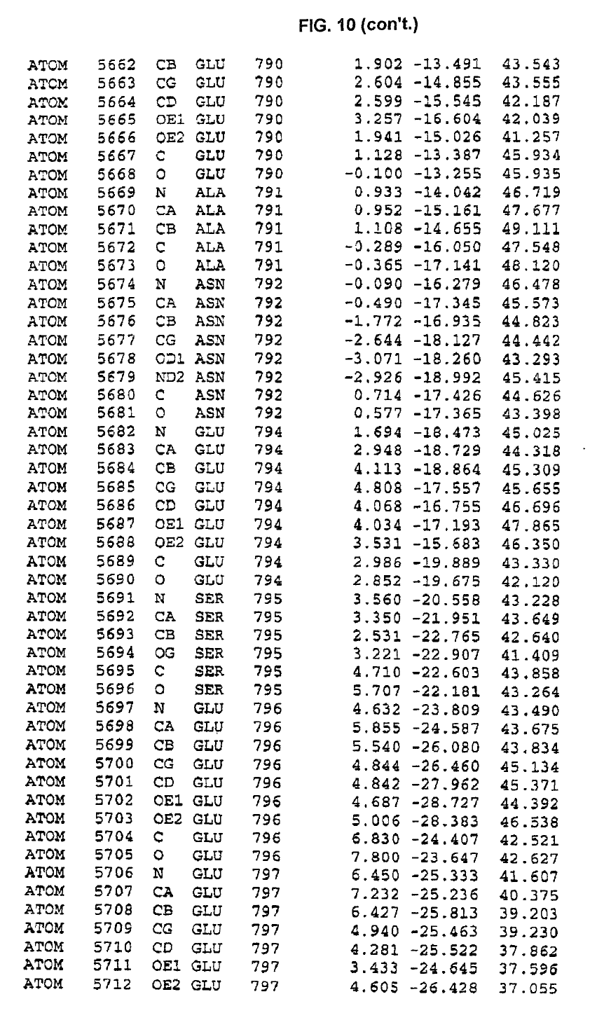

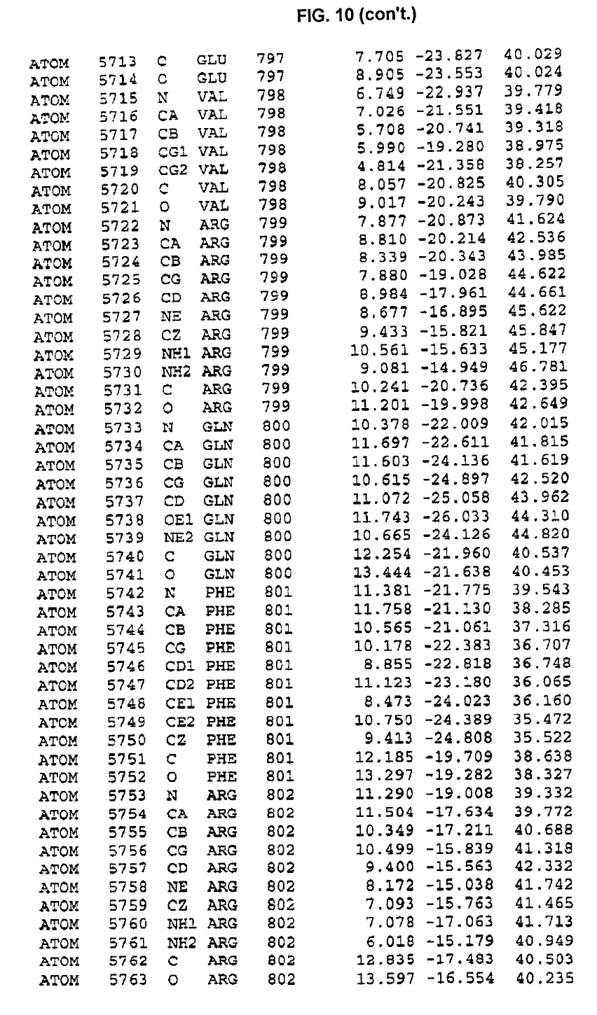

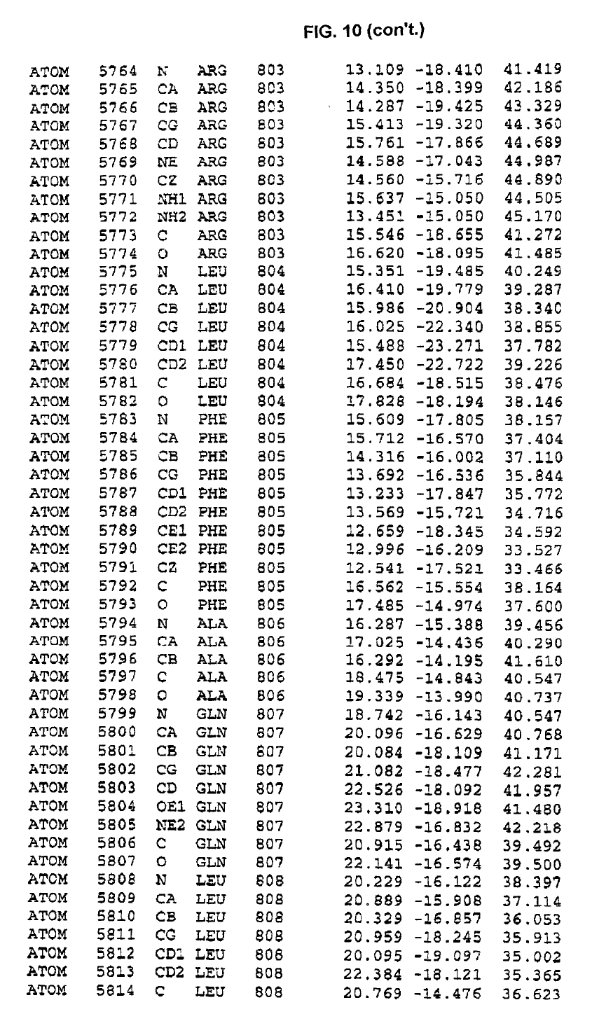

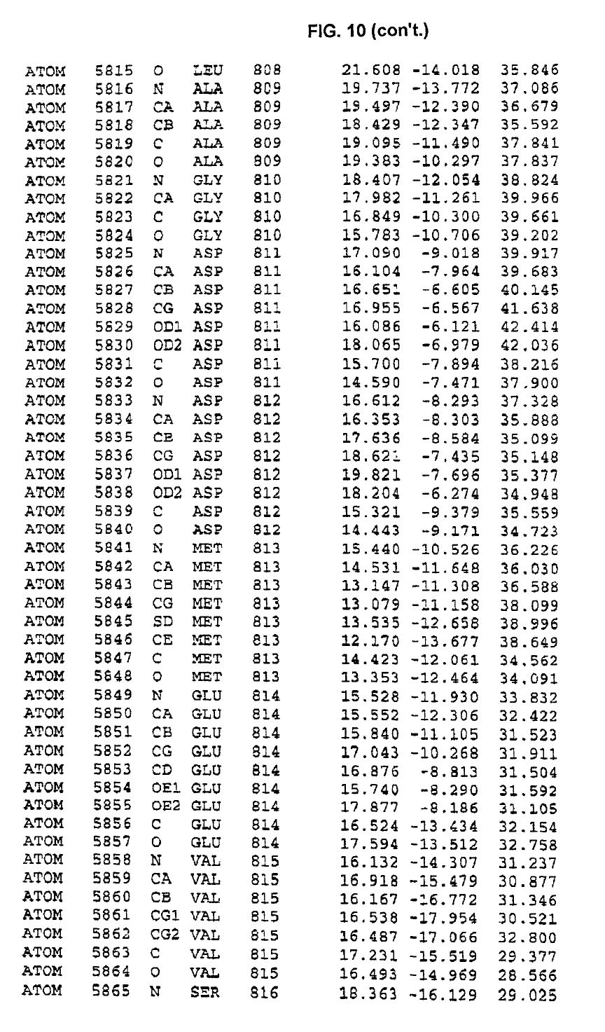

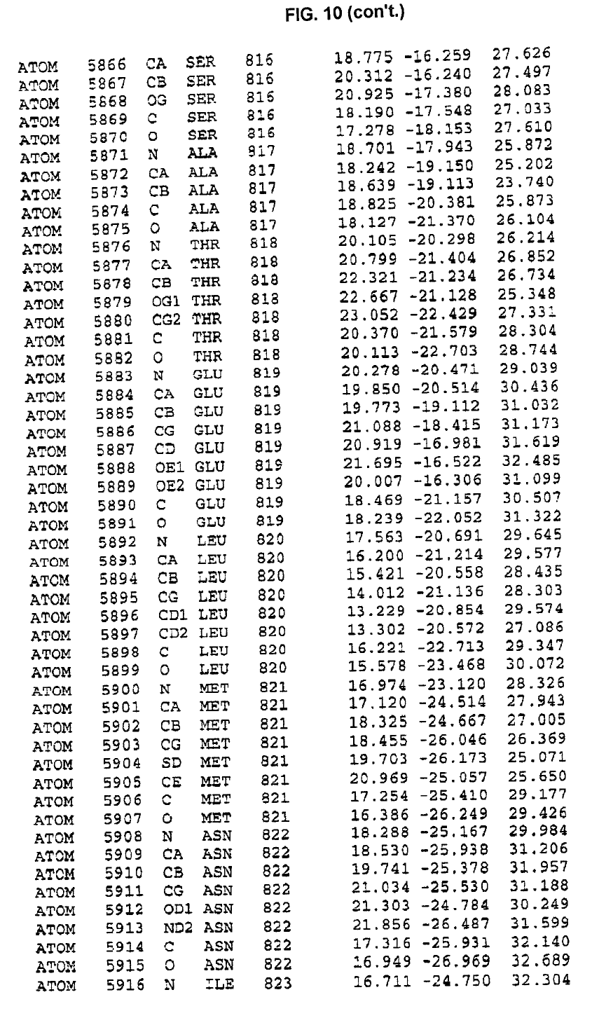

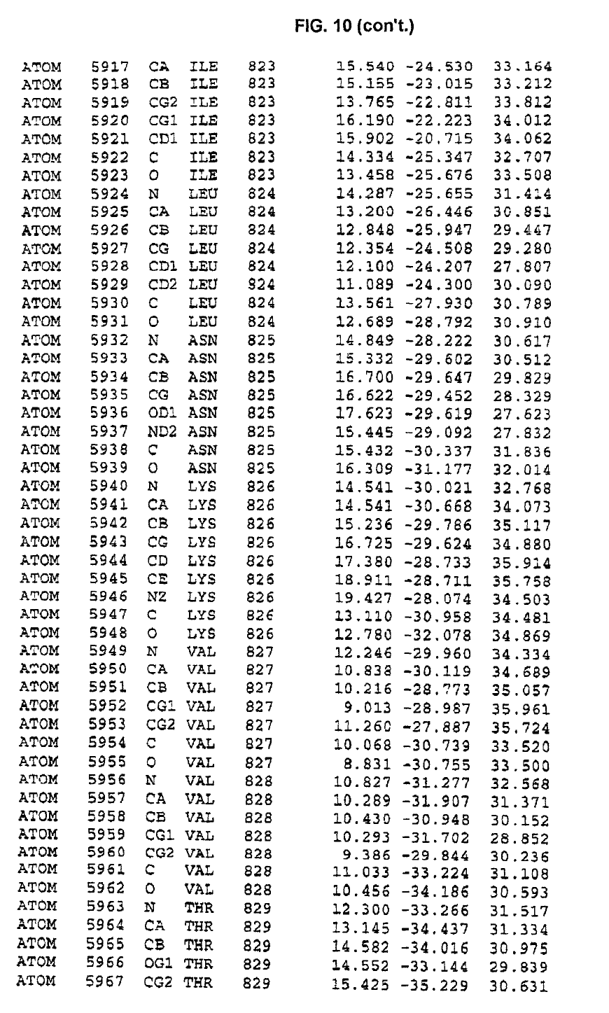

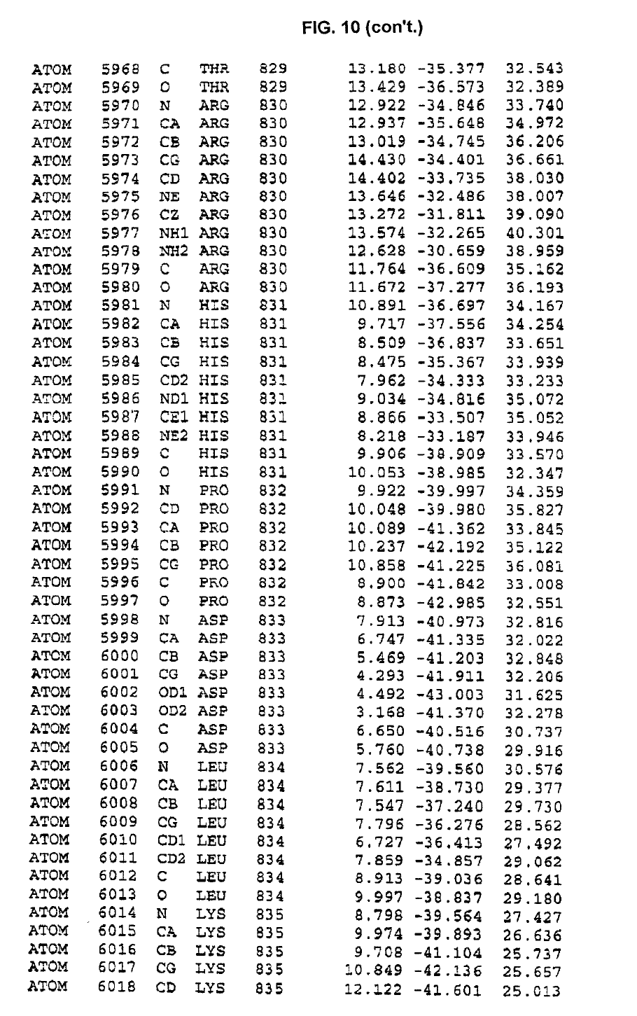

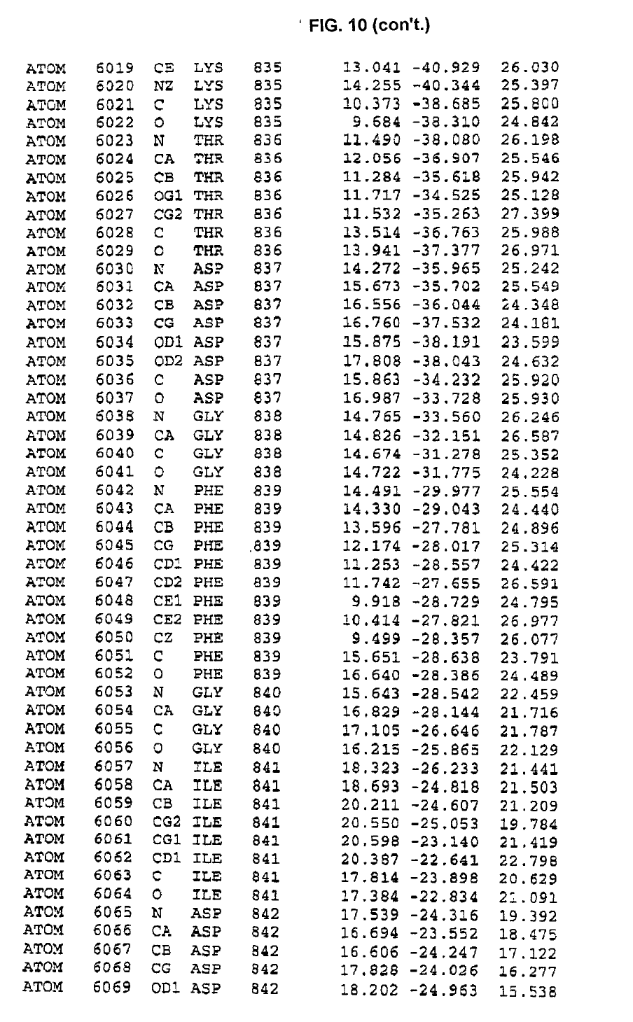

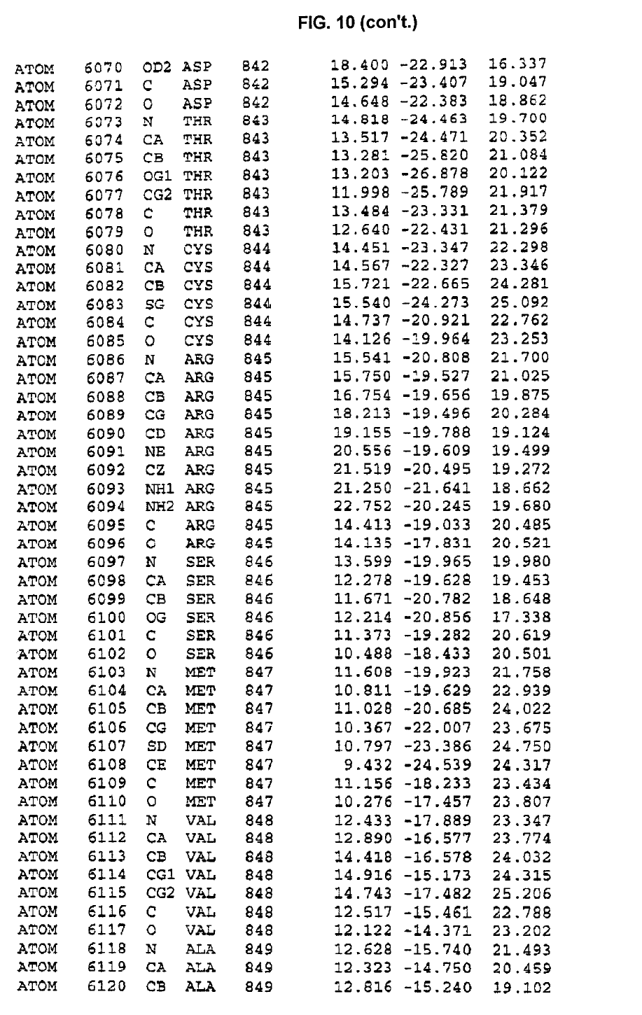

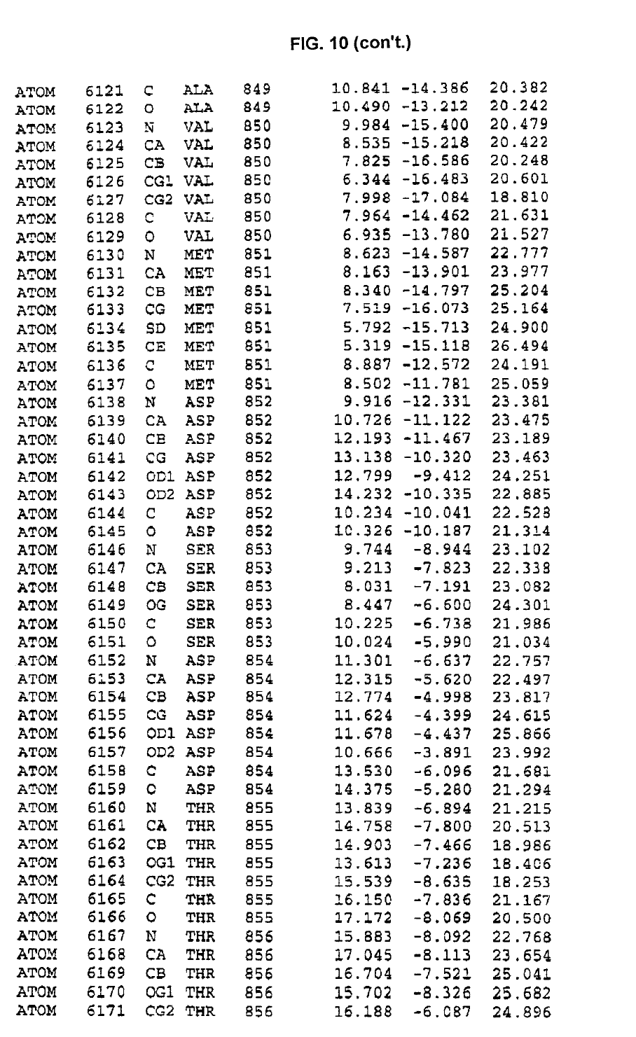

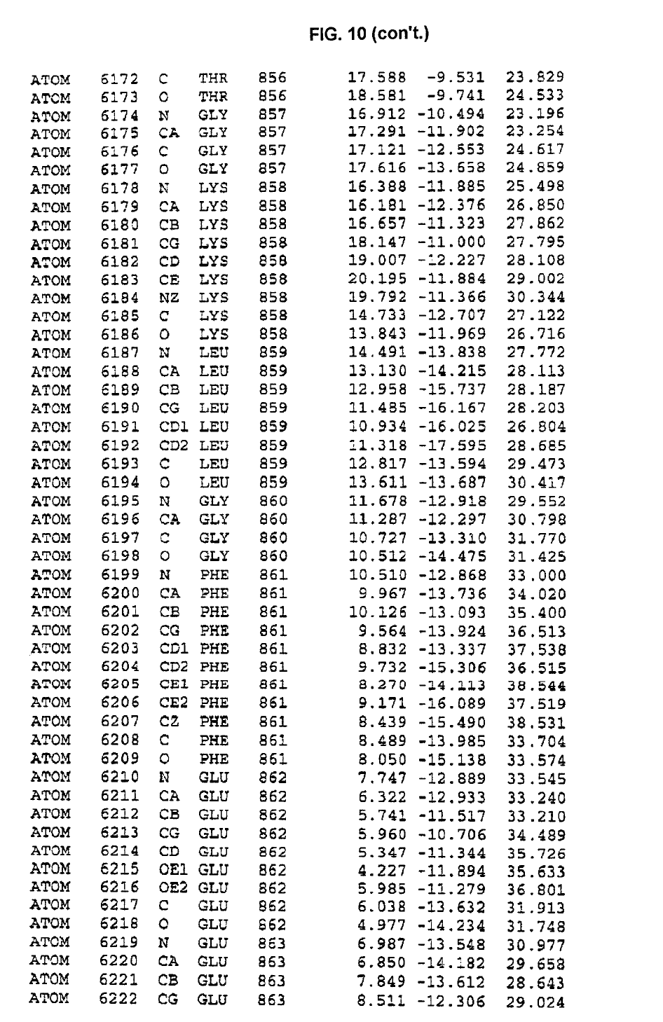

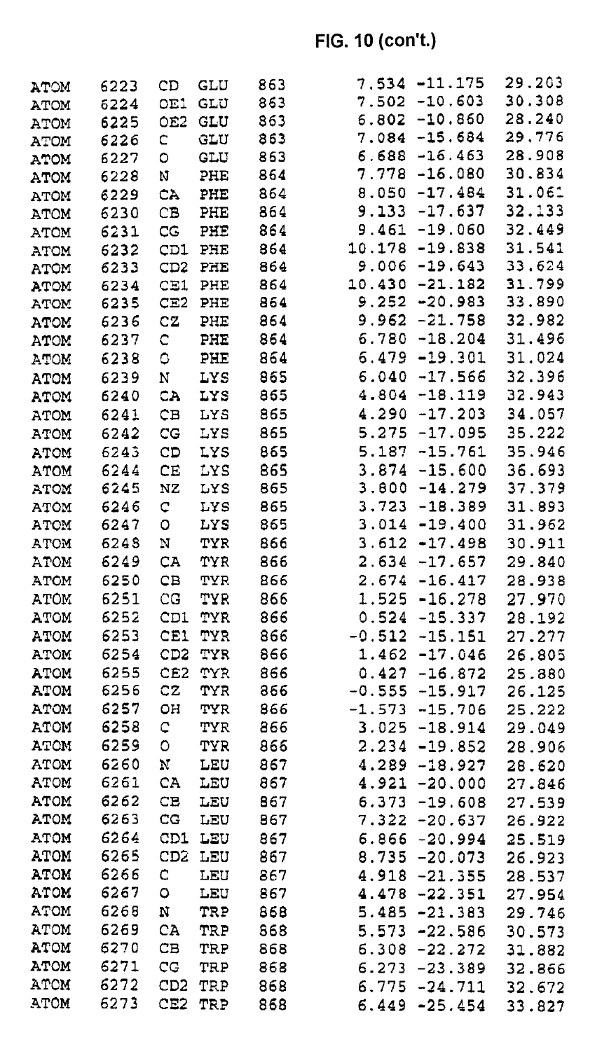

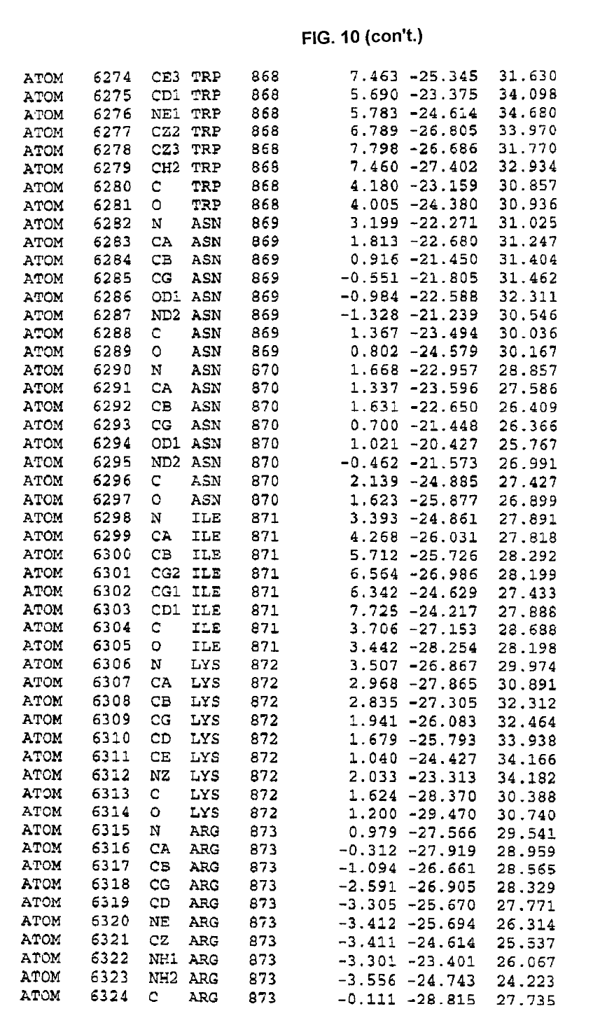

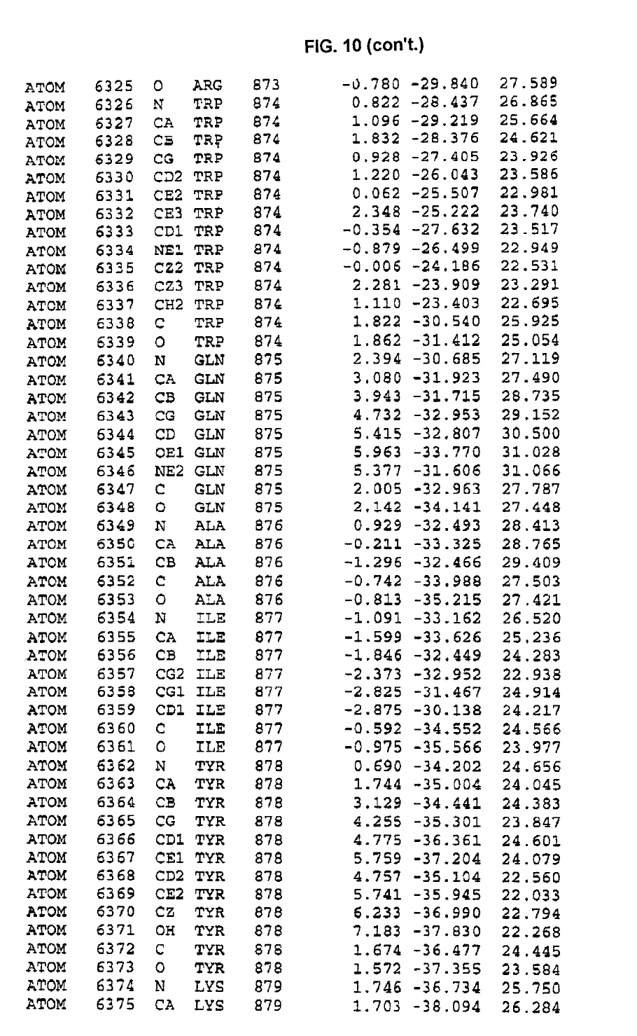

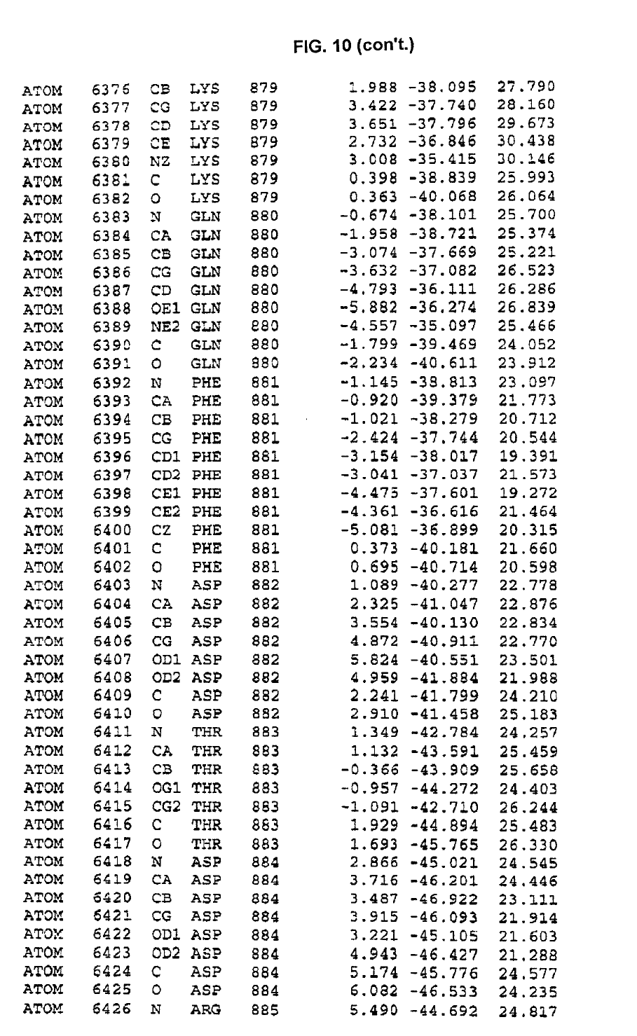

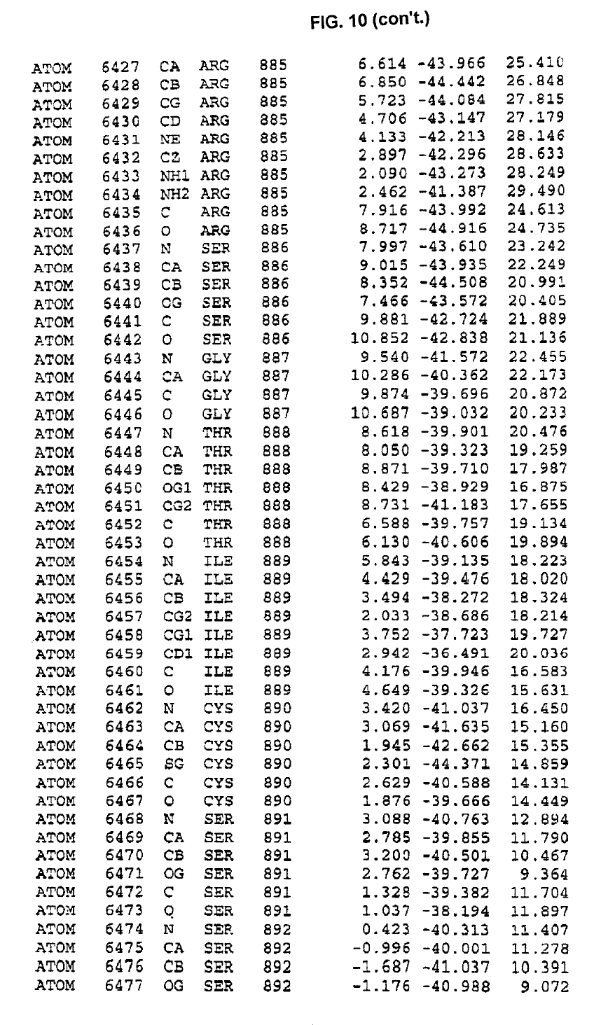

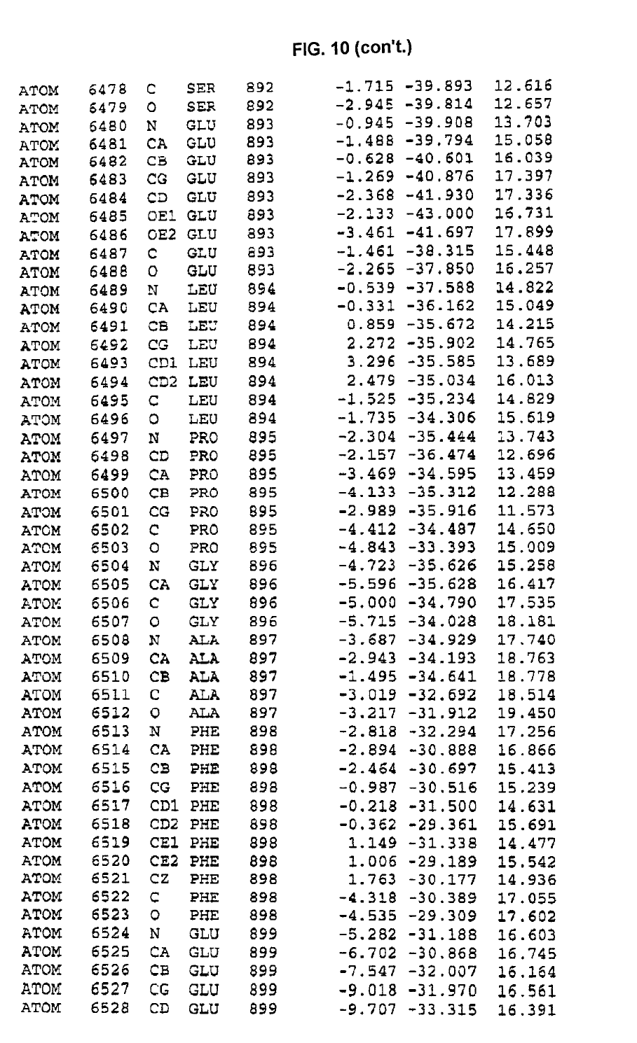

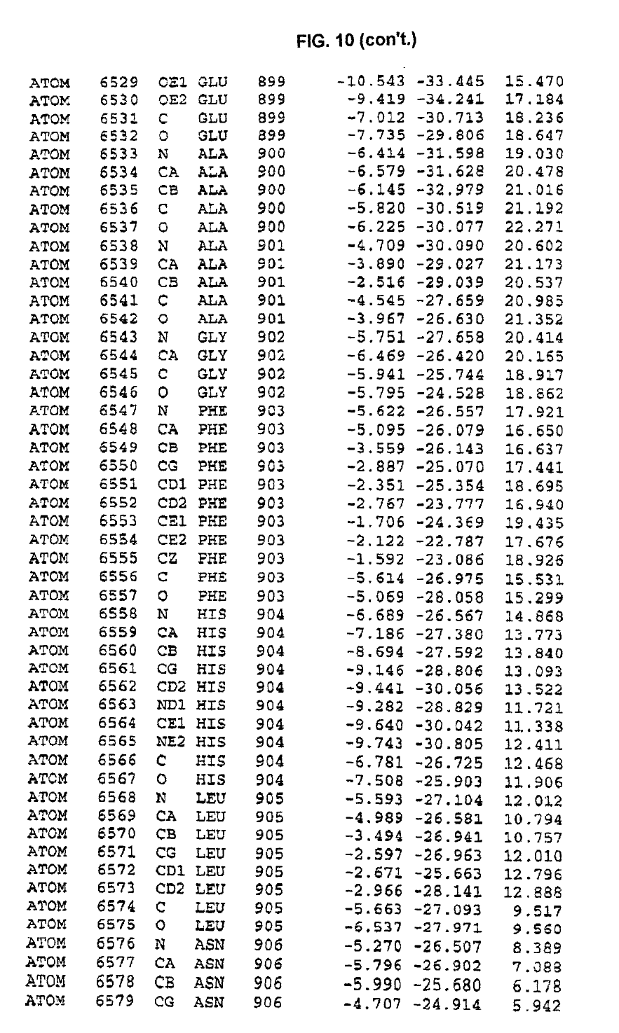

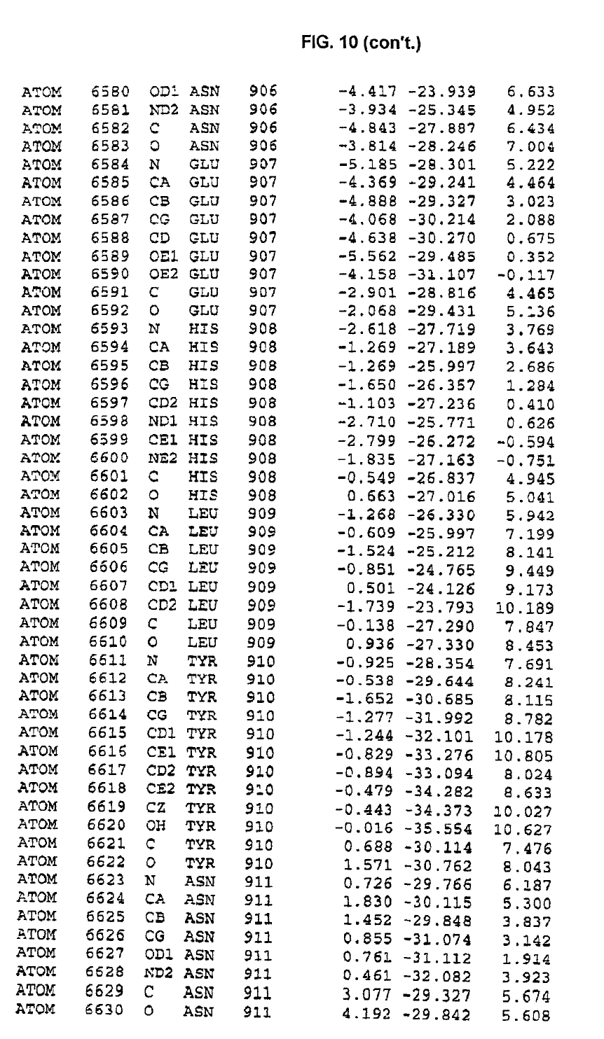

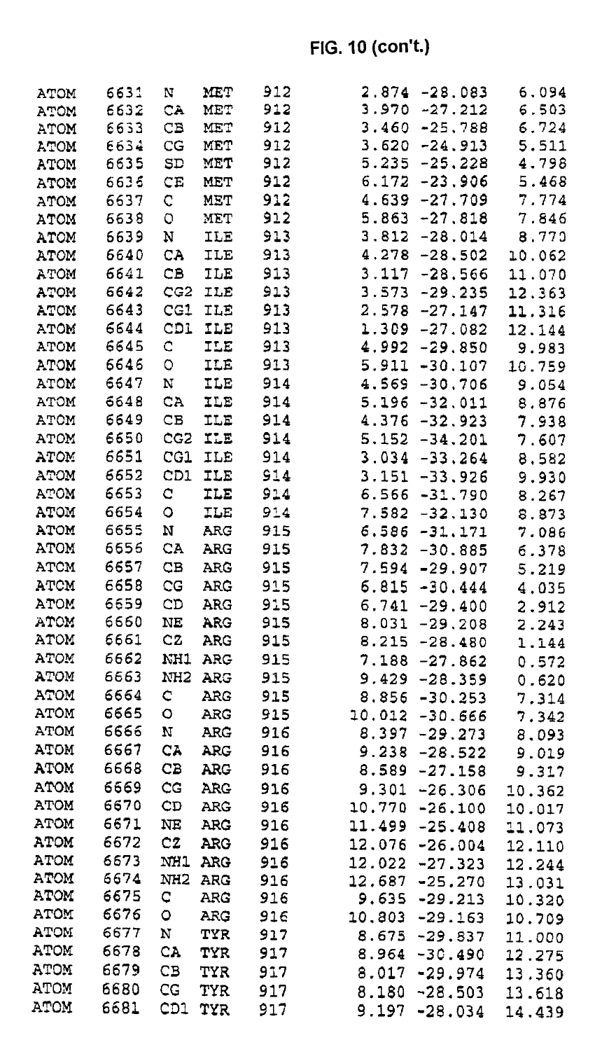

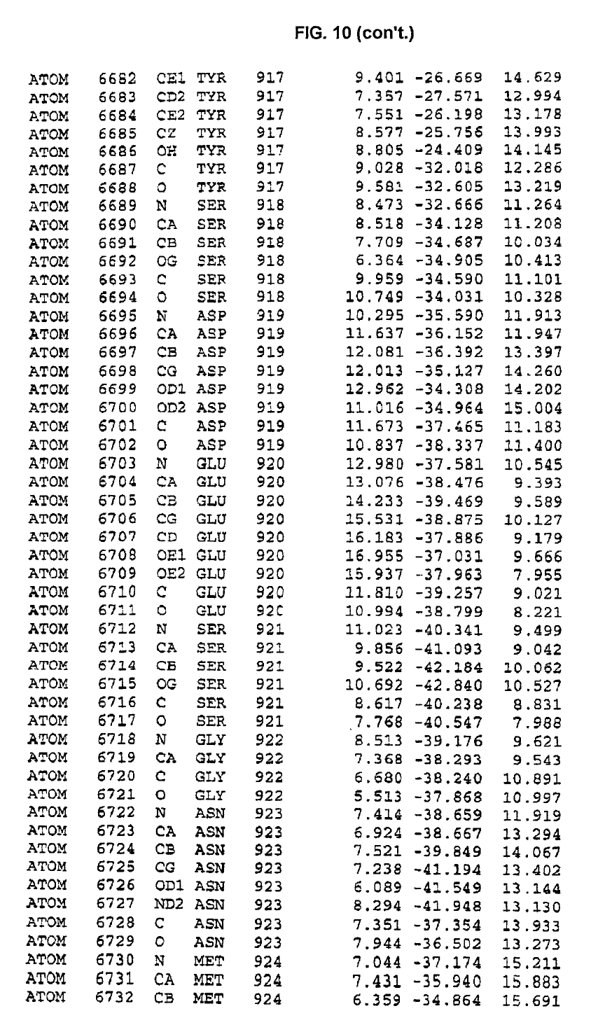

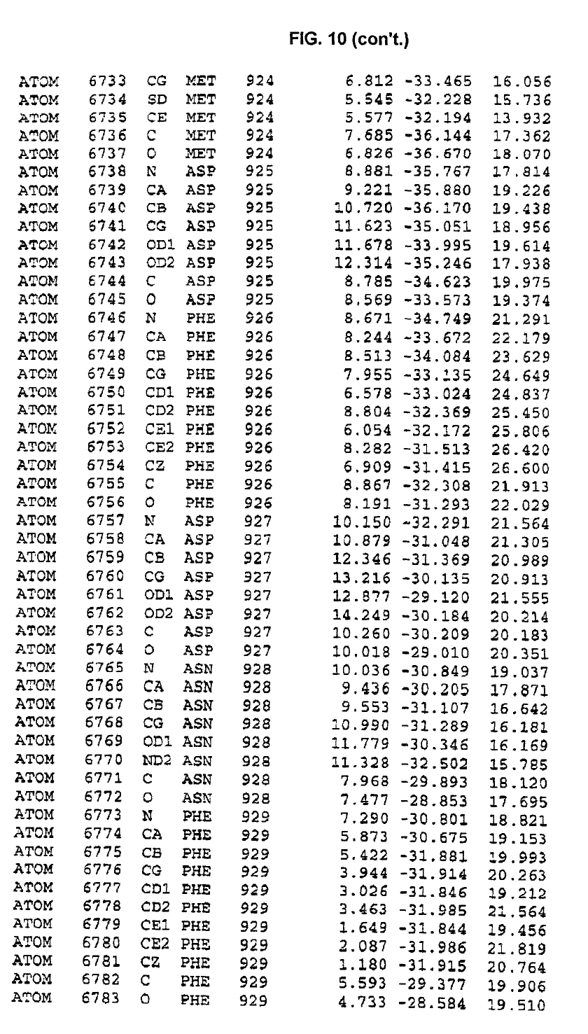

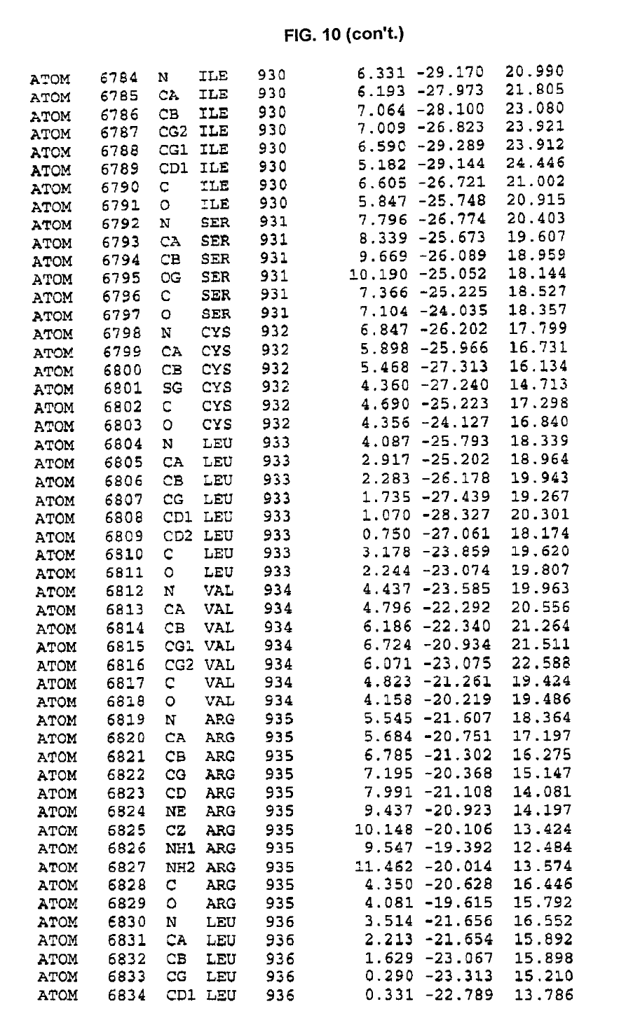

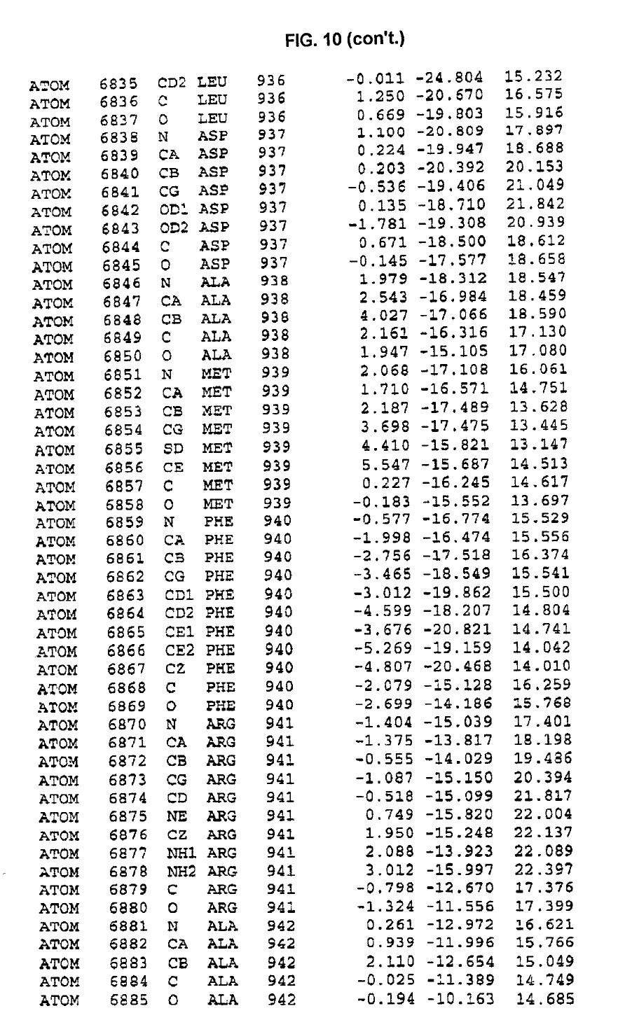

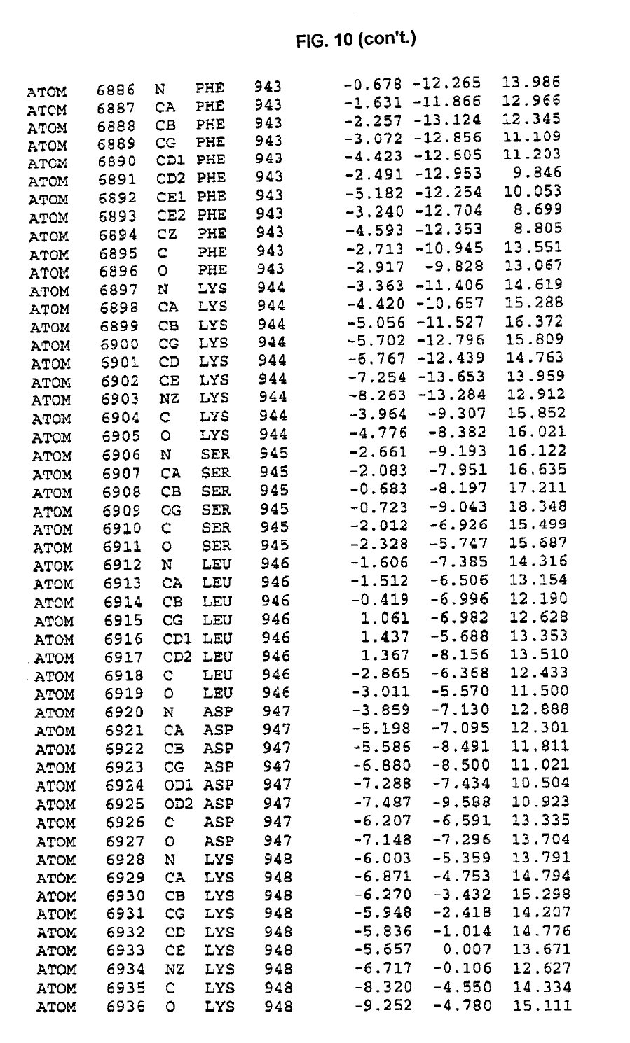

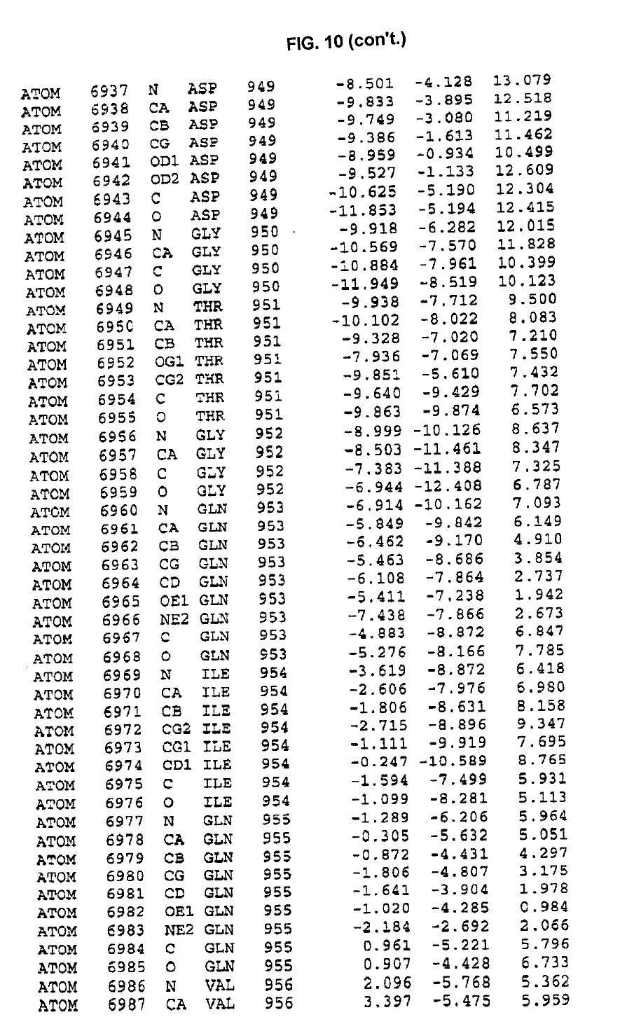

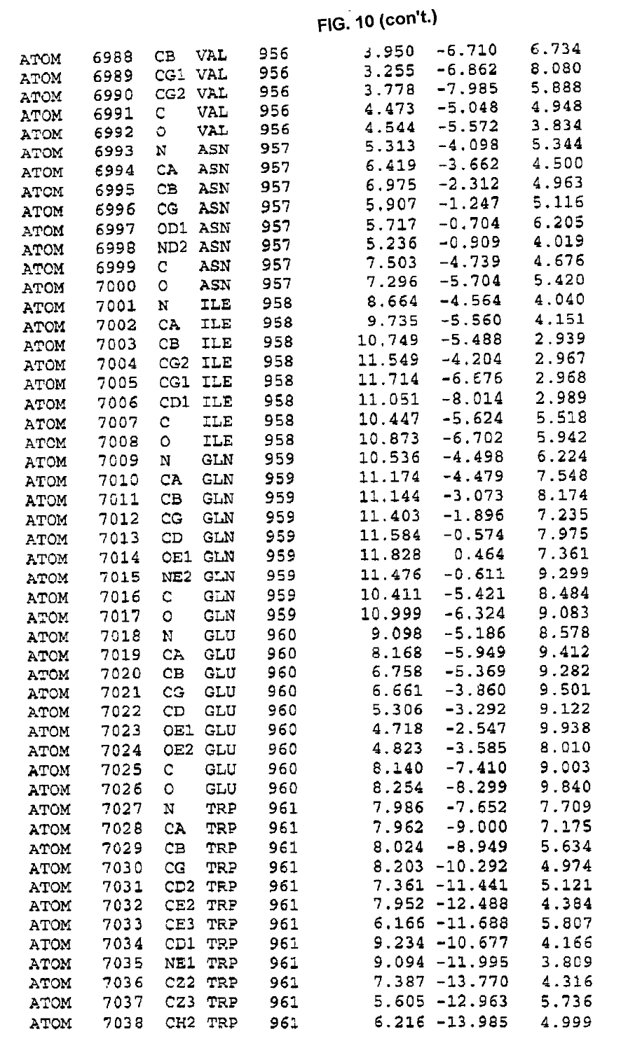

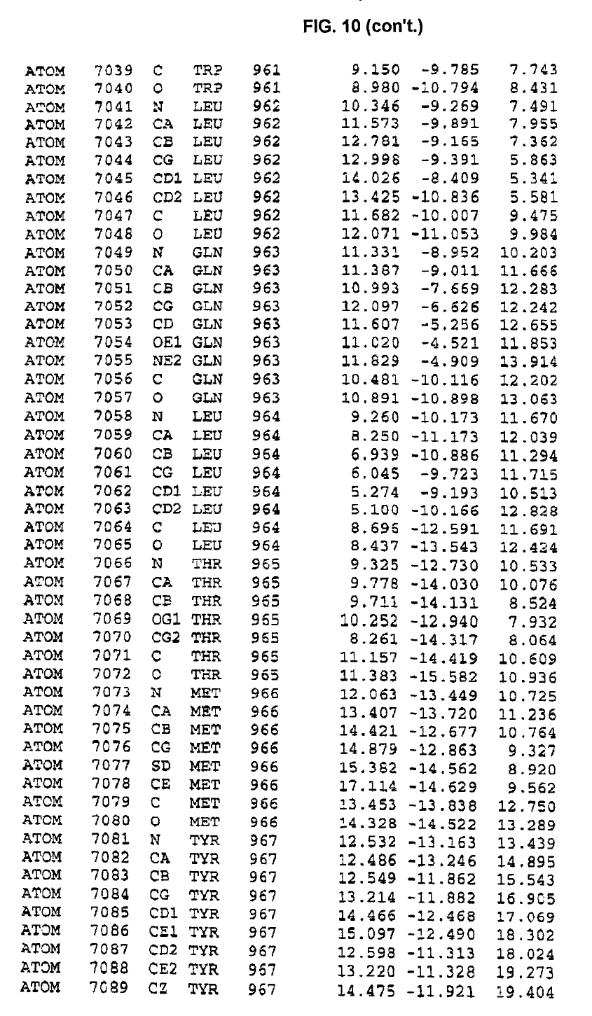

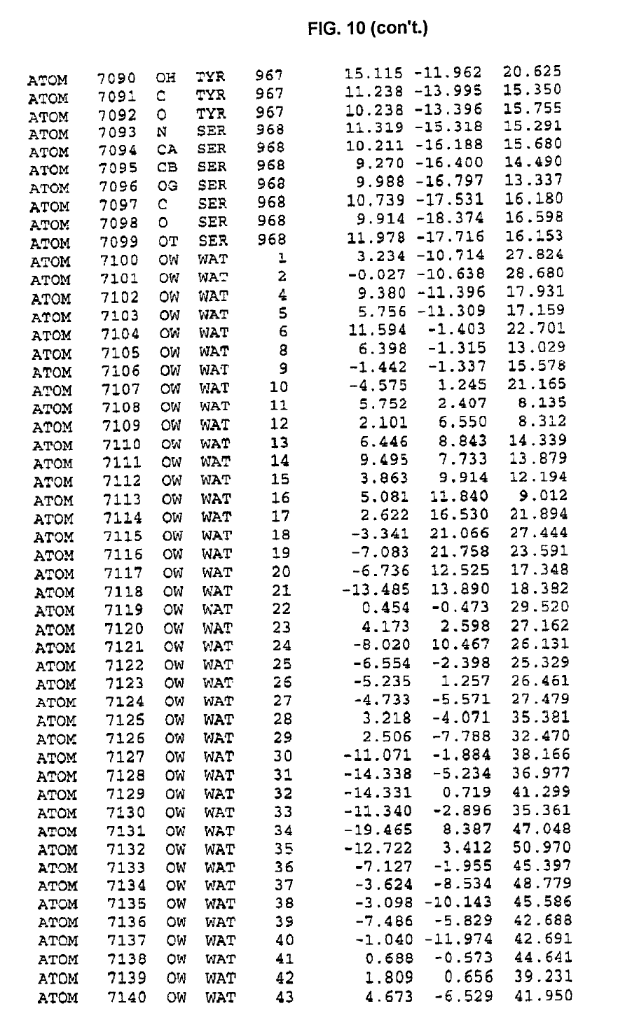

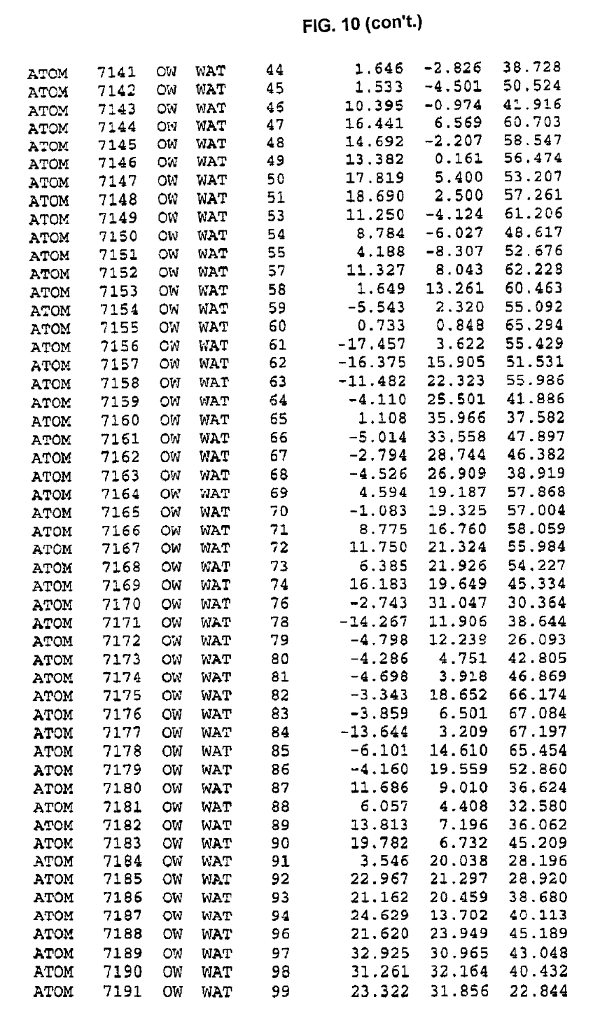

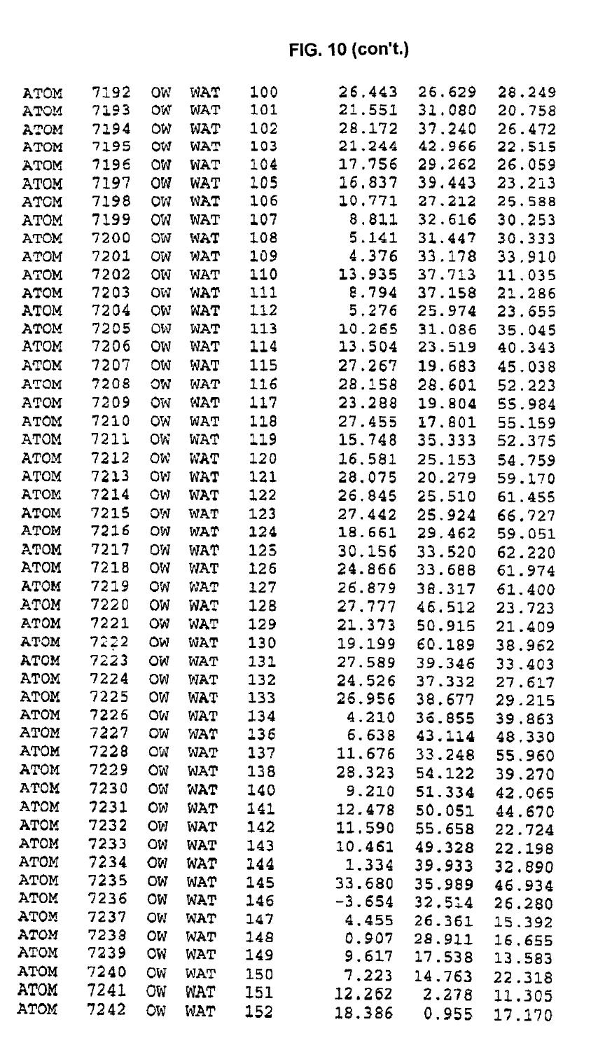

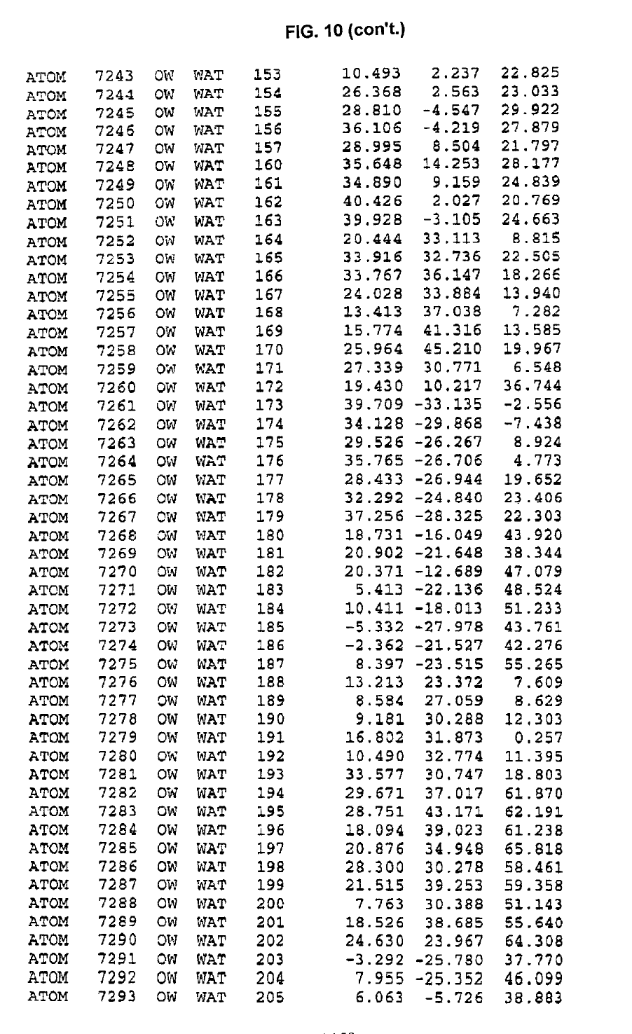

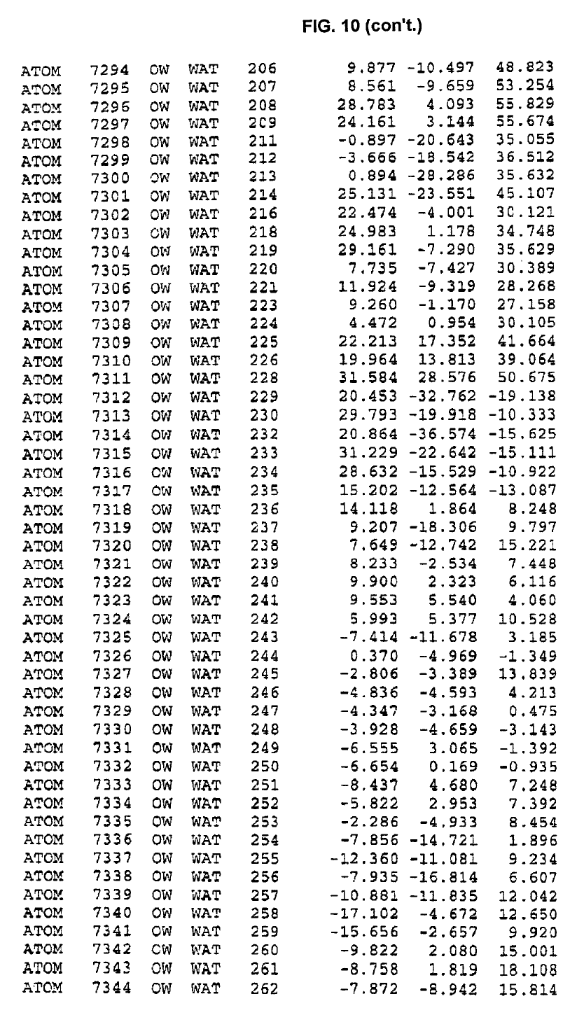

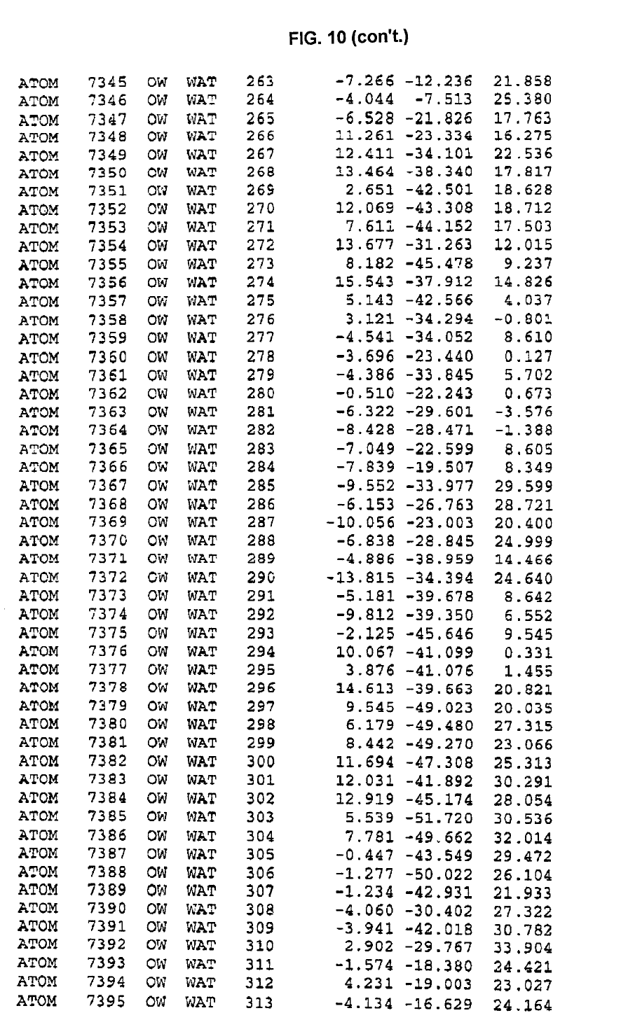

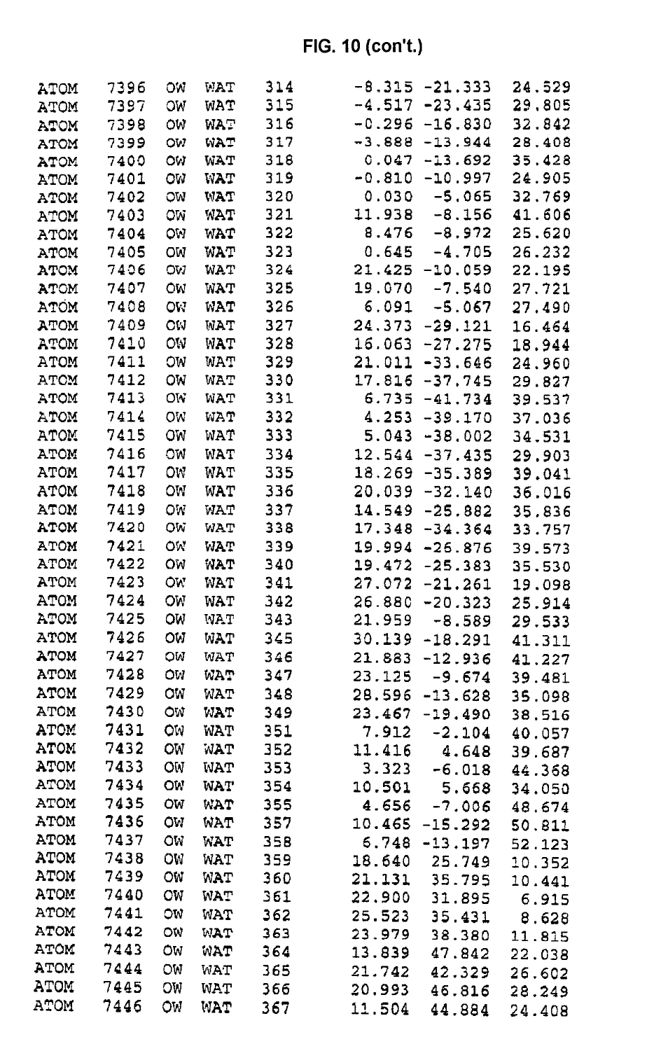

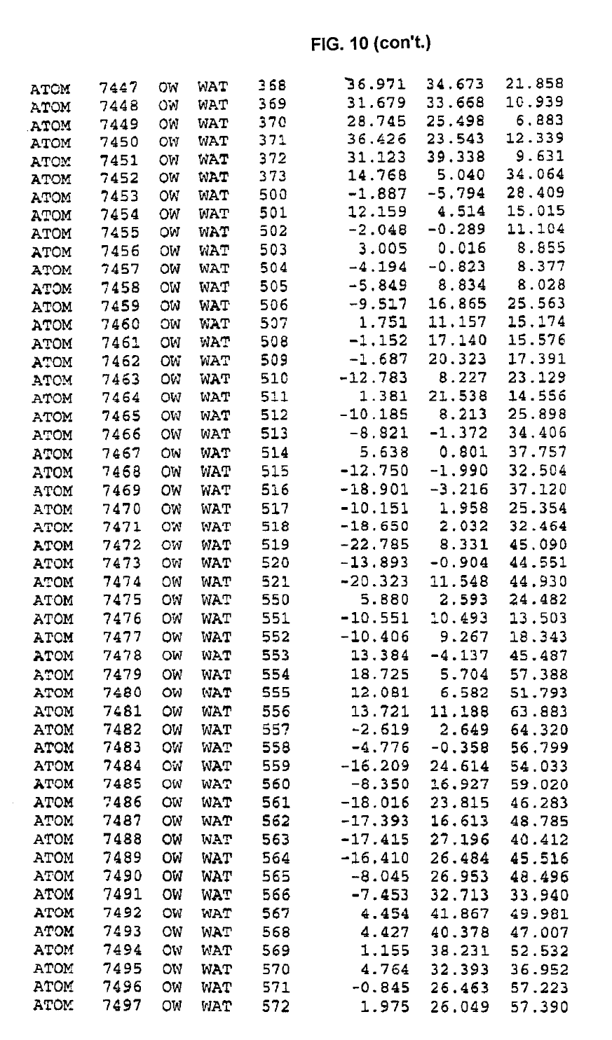

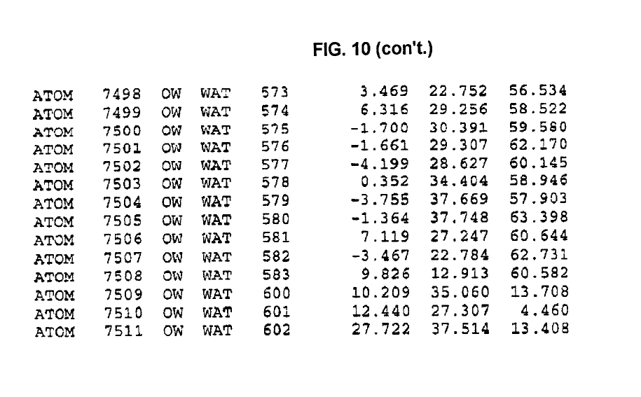

In a further preferred embodiment, the subdomain which participates in the calpain reaction and is contained in the polypeptide sequence whose spatial structure is present in the crystal form has a three-dimensional appearance as shown by the structural coordinates according to FIG. 10. FIG. 10 shows the structural coordinates for the crystal form of the subdomains IIa and IIb of human m-calpain. Even more preferred is a crystal form of a heterodimer comprising large and small calpain subunits, it being possible for the polypeptides (1) and (2), which correspond to the small and large subunit, respectively, to be represented by a crystal form according to the structural coordinates of FIG. 10.

Crystal forms of the type according to the invention are preferred in particular when they have a resolution of less than 3.5 Å, preferably less than 3.0 Å and very particularly preferably less than 2.5 Å.

The present invention furthermore relates to compounds which can bind as ligands to a spatial form or preferably crystal form according to the invention, and crystals according to the invention which have such microscopic spatial or crystal forms. These ligands will typically have a property which makes it a pseudosubstrate, substrate, activator or inhibitor and are distinguished by the fact that they have steric properties and/or functional groups which are capable of interacting with the main and/or side chains of the catalytic subdomain(s) or of a sequence segment relevant for regulation of the catalytic subdomain(s). These interactions of at least one ligand with segments of the small and/or large subunit of calpain may give rise to conformational changes which may affect the proteolytic activity of the calpain, in particular inhibit its catalytic activity. The ligand must accordingly have steric properties or an interaction potential which is complementary to the main and/or side chains of the calpain in its spatial or crystal form or its steric properties (for example for clefts or non-compactly filled regions in the interior of the protein), as prescribed, according to the invention, by the spatial form or preferably the crystal form.

Particularly preferred ligands here are, however, those which bind to the domain III of calpain and especially interact with the external acidic loop β2III/β3III and/or structurally with the acidic loop of neighboring amino acids. This is in particular (a) the region with the amino acids 392 to 403, which comprises altogether ten negatively charged amino acids, i.e. aspartates or glutamates; (b) opposite this region, the amino acids of the amphipathic helix α7II; (c) the sequence segment which is formed by the amino acids K354 to K357; and/or (d) that of the amino acids K505 and K506.

A ligand binding to this loop would typically have the form such that it has at least one positive charge and/or at least one positive partial charge and docks by means of this charge structure with the negatively charged acidic loop. In this way, a ligand structured in this manner prevents the interaction of the acidic loop β2III/β3III with the amphipathic helix α7II, which comprises the basic amino acids K226, K230 and K234 of the large subunit. An at least partially positively charged ligand could compensate the negative excess charge present on the above-mentioned loop and in the end act as an activator of the catalytic activity of the calpain. In this way, the positively charged helix of the catalytic subdomain IIb is not detached from its compact fold by the negatively charged acidic loop of the domain III by the formation of salt bridges, and the catalytic activity is therefore maintained.

A compound according to the invention which binds as a ligand in this structural region of calpain should be designed with respect to its chemical, geometric and/or physical properties to correspond to the structural requirements of a crystal form according to the invention. Moreover, it may be present covalently or noncovalently in a crystal form according to the invention with the at least one polypeptide.

In particular, it is necessary to observe one or more general conditions which are mentioned below for certain functional groups of amino acid side or main chains, also taking into account specific distances to or between these functional groups, for the design of compounds according to the invention. Between the functional group K226, namely the atom NZ, and the functional group D400, i.e. the two oxygen atoms, there is a distance of 3.32 and 3.8 Å, respectively, in a crystal form according to the invention. Furthermore, there is an interaction between the lysine K230 of the amphipathic helix and D397, namely between the atom NZ of K230 and the two oxygen atoms of the aspartate D397 at a distance of 3.73 and 3.66 Å, respectively. In addition, the side chain of K234 (NZ) interacts with an oxygen atom E504 (OE1). A further contact exists between K354 (NZ) and the atoms OE1 and OE2 of E504 (2.7 Å and 3.36 Å, respectively). The amino acid K505 (NZ) is also associated with the atom OE1 of E396 via a salt bridge (4.56 Å). Moreover, there is an interaction between K506 (NZ) and E393 (OE1) (distance of 2.86 Å) and E393 (OE2) (distance equal to 4.93 Å). Furthermore, interactions are to be observed between the amino acid K357 (NZ) (from the structural region (c)) and the amide oxygen from the main chain of E504 (3.68 Å).

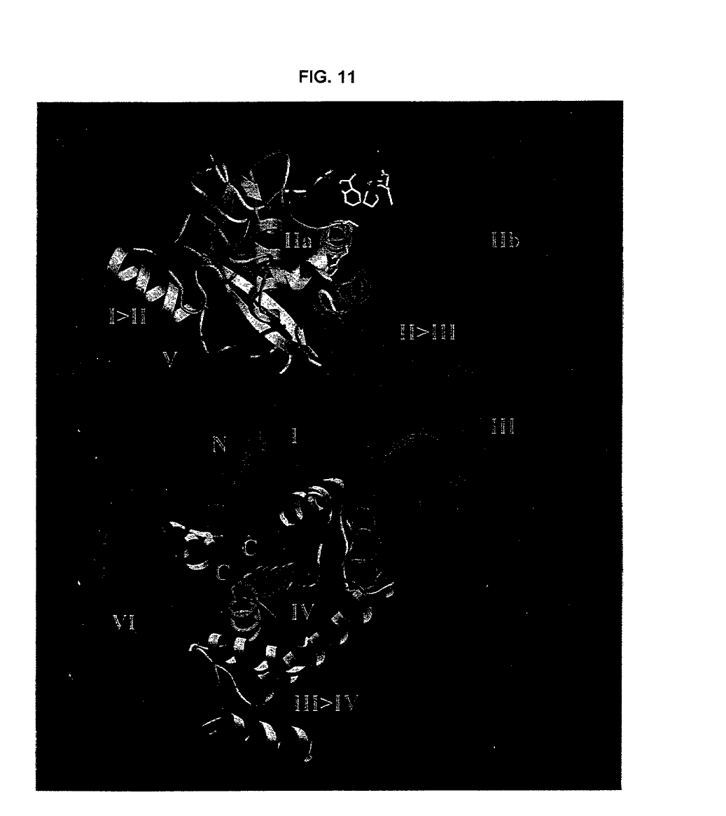

A compound according to the invention which binds to the domain III of calpain according to a crystal form according to the invention or to a crystal according to the invention which has such crystal forms according to the invention preferably has the character of a Ca²⁺ analog. In the end, the effect of the positively charged calpain activator leads to permanent conformational proximity of the catalytic subdomains IIa and IIb without formation of a cleft, as is evident in FIG. 11, the prerequisite for the catalytic activity of calpain.

Compounds which bind to the active center of calpain, as present after association of the two subdomains IIa and IIb, and which may act there as inhibitors of the catalytic reaction typically interact with at least one of the following amino acids Gln99, Cys105, Ser241, Asp243, Asn286, Gly261, His262 and/or Trp288.

In order to provide an inhibitory compound according to the invention which conserves the inactive conformation of the two subdomains IIa and IIb and hence completely or partially fills the cleft present between these two subdomains, its physiochemical and/or geometric character will preferably fulfill one or more of the following boundary conditions determined by the three-dimensional structure of a calpain. Particularly preferred are contacts with one or more of the following amino acids (side or main chain): Gln99, Cys105, Ser241, Asp243, Gly261, His262, Trp288, Arg94, Asp96, Cys98, Trp106, Ala109, Thr200, Ile244, Lys260, Asn286, Glu290, Leu108, Thr201, Phe204, Trp214, Asp249, Lys257, Ala263, Tyr264, Glu292, Ser336 and/or L338 (according to the numbering scheme for human m-calpain) or corresponding amino acids of other calpain forms.

Inhibitory compounds of the inactive calpain form with a cleft are very particularly preferred when, for example, they are involved in interactions with Q99 with their two functional groups on atom NE2 and on atom OE1. Both atoms can be involved in hydrogen bridges with the inhibitor, in which case the length of the hydrogen bridge bond is typically greater than 2.0 Å, preferably greater than 2.2 Å, the geometrical requirements for hydrogen bridge bonds (e.g. directionality) being taken into account. Furthermore, such an inhibitor can preferably interact, for example, with the amino acid C105. Here, the carbonyl function of the backbone and the hydrogen or the free electron pairs on the sulfur SG of Cys105 may be particularly mentioned. The corresponding functional groups of the inhibitor are arranged a distance greater than 2 Å and typically less than 3.5 Å away from the above-mentioned regions of Cys105.

In addition, the preferably inhibitory ligand binding in the cleft can interact with the side chain of the amino acid S241, preferably with the hydroxyl group on the serine residue. This hydroxyl group, too, can form, for example, at least one hydrogen bridge bond to the inhibitor in a directional manner typical for hydrogen bridge bonds, with a distance between 2.0 and 3 Å. For example, interactions of the inhibitor via salt bridges with the carboxyl group on the side chain of the amino acid D243 are also preferred. Typically, the inhibitor will have at least one positive charge and/or partial charge with respect to this carboxyl group, at a distance greater than 2 Å.

It is also possible, for example, for there to be a contact with a carbonyl group of amino acid G261, preferably through a corresponding chemical group of the inhibitor, which should typically be in the form of a hydrogen bridge bond with a distance of at least 2.0 Å. Typically, the side chain of the amino acid W288 will also be involved in an interaction with an inhibitory ligand. Here, it is possible both for the hydrogen present on the indole nitrogen to form a hydrogen bridge bond with a distance of typically more than 2.0 Å with the inhibitor and for the inhibitor having a preferably aromatic structure which is typically annular in this respect or such a structural element to interact with the aromatic indole group, for example via so-called stacking. This is an arrangement of, for example, aromatic ring systems which is stacked in parallel.

Preferably, the inhibitor will form, for example, hydrogen bridges to the hydrogen on one of the nitrogen atoms of the histidine ring of H262. With the histidine ring, too, a hydrophobic interaction via the above-mentioned stacking with annular fragments of the inhibitor is possible. In this case, the annular systems are typically parallel to one another.

If required, the amino acid R94 with the positive charge on the nitrogens of the arginine can interact with the inhibitor. Here, a hydrogen bridge and/or a salt bridge with the corresponding functional group of the inhibitor with a typical distance of between 2.0 and 3.8 Å may be preferred. Likewise, the carbonyl function of T95 can interact with the inhibitor with a directional hydrogen bridge, or the carboxyl function of the side chain of D96 can preferably form at least one salt bridge with the inhibitor.

C98 is preferably likewise involved in interactions with the functional groups of the inhibitor. Both the hydrogen on the sulfur atom and the free electron pairs on the sulfur may be suitable for such interactions. Furthermore, for example, W106 preferably interacts with the inhibitor. Possible forms of the interaction correspond to those which have been described above for W288, in particular there is the possibility here too of an interaction between aromatics, in particular by the method of stacking.

Two further interactions on the basis of which the inhibitor can bind in the cleft between the two subdomains may be attributable, for example, to contacts with carbonyl groups of the two amino acids L108 and A109, for example likewise in the form of directional hydrogen bridges.

Hydrophobic interactions of the inhibitor with amino acids in the region of the cleft of a crystal form according to the invention may be specified for the side chains of A109 and A263. Furthermore, a hydrophobic cluster is formed in the cleft between the two subdomains IIa and IIb by the side chains of the amino acids F204, L338 and the hydrophobic segment of the side chain of T201. An inhibitor is preferably formed in such a way that it has a hydrophobic segment which can interact with the above-mentioned hydrophobic groups.

In a crystal form according to the invention, the side chain of the amino acid I244, which can likewise preferably undergo a hydrophobic interaction with the inhibitor, is present opposite the helix α2II. From the subdomain IIb, the side chain of the amino acid K260 can project into the gap formed between the two subdomains, so that advantageously, for example, a salt bridge between the inhibitor and the positive charge on the NZ atom of K260 will be present. Here, the inhibitor would therefore preferably have a negative charge and/or partial charge on the position complementary to the NZ atom.

A particularly preferred point of attack of the inhibitor for interactions would be an interaction with the side chain of the amino acid residue N286 involved in the catalytic reaction. Here, for example for realizing an inhibitor, it would be possible to form directional hydrogen bridge bonds with distances between 2.0 and 3.0 Å, with the carbonyl and/or the NH₂ group of N286. The side chain of the amino acid E290 projects into the gap of the inactive calpain between the two subdomains, with negative charges which can preferably be compensated according to the invention by corresponding positive charges of an inhibitor which are present in the spatial vicinity.

A further preferred boundary condition for the design of an inhibitor according to the invention may advantageously constitute the hydrophobic side chain of the amino acid L108. Typically, an inhibitor can then likewise have hydrophobic fragments at a complementary point. Furthermore, the inhibitor may be involved in interactions with the tryptophan residue of W214, for example through hydrogen bridges at the indole nitrogen or the above-mentioned aromatic interaction. In addition, there may also be a contact between the inhibitor and the carboxyl group of D249, preferably in the form of a salt bridge between, for example, a positive charge of the inhibitor and the carboxyl group.

A salt bridge can advantageously also be present from the positive charge on the NZ atom of K257 to a corresponding negative charge and/or partial charge of the inhibitor. In the case of inhibitors which preferably dock in the outer region of the cleft, close to the complex surface, an interaction of functional groups of the inhibitor with the negative charges of E292 would be desirable. Here too, the inhibitor would form a complementary positive charge and/or partial charge in the form of a salt bridge with E292. If an inhibitor with the property is to be used at the outer surface of the calpain complex in the region of the cleft, an inhibitory ligand may also be, for example, in contact with the amino acid Y264. This can advantageously result in a blockage of the amino acid N286 involved in the catalytic reaction and H262. The tyrosine residue Y264 can typically undergo interactions with the inhibitor, both via at least one hydrogen bridge bond, which starts at the characteristic hydroxyl group, and, as a result of its aromatic character or its hydrophobic character, with corresponding functions on the inhibitor. Where the inhibitor projects deeply into the cleft, an interaction with the amino acid residue S236 of the subdomain IIb, in particular a hydrogen bridge therewith, would be particularly desirable.

In particular, the present invention relates to those ligands which can bind to a spatial or crystal form which is represented by the structural coordinates according to FIG. 10.

Crystal forms according to the invention are also distinguished by the fact that, as a three-dimensional structure characterized by structural coordinates for each individual atom forming the structure, they are part of a symmetrical arrangement in a crystal. It is preferable if a crystal form according to the invention which contains at least one polypeptide having at least one catalytic subdomain, after superposition with the structural coordinates listed in FIG. 10 for the at least one subdomain involved in the catalytic reaction, has a standard deviation (rms) of less than 2.5 Å, preferably of less than 2 Å.

The present invention furthermore relates to crystals which have crystal forms as claimed by claims 1 to 23, arranged according to laws of symmetry. These include crystals of all those crystal forms which are disclosed according to the present invention. These may be natural crystals, derivative crystals or cocrystals. Natural crystals according to the invention essentially have a symmetrical arrangement of at least one polypeptide which contains at least one subdomain involved in the catalysis of the calpain reaction, optionally in combination with calcium ions, as part of a crystal form. Here, the catalytic subdomain contained in the crystallized polypeptide may be both active and inactive mutants thereof. Inactive mutants are very particularly preferred when they essentially retain the structure of one subdomain or of both subdomains of calpain.

The present invention furthermore relates to methods for identifying a compound which has the property of acting as a substrate, pseudosubstrate, activator, inhibitor or allosteric effector of calpain or of a mutant of calpain, in particular human calpain, very particularly preferably human m-calpain. Such a method is particularly preferred when the compound binds with ligand functions to a structural region of one of the two catalytic subdomains, in particular in the region of the active reaction center. In such a method, (a) a crystal form as claimed in any of claims 1 to 23 is obtained, with the crystal formed being present in the form of its structural coordinates, (b) the structural coordinates of the crystal form are represented in three dimensions, (c) and the steric properties and/or functional groups of a compound with a ligand function are chosen so that interactions between the compound and the main and/or side chains of the polypeptide which forms the active center are possible. Ligands suitable according to the invention, in particular suitable inhibitory ligands which conserve the inactive calpain structure with a cleft between the subdomains, are determined on the basis of these interactions.

The representation of the structural coordinates of a crystal form according to the invention is preferably effected by graphical plotting with the aid of corresponding computer programs on a computer screen. On the basis of the complementary arrangement, based on potential ligands, of the main and side chains of the crystal form, for example in the active center of a calpain, it is possible, by a nonautomated method, to identify ligands suitable according to the operator's experience and having corresponding chemical and/or steric properties, to design said ligands on the screen and finally to simulate their binding behavior.

Preferably, however, the choice of suitable ligands is made by an automated method, by searching through computer databases which contain a large number of compounds. The search is based on the prior characterization of geometric, chemical and/or physical properties for the desired calpain ligands. Databases to be searched through contain naturally occurring as well as synthetic compounds. For example, the compounds stored in the CCDC (Cambridge Crystal Data Center, 12 Union Road, Cambridge, UK) may be used for such a search. However, the databases available from Tripos (cf. citation, loc. cit.), namely Aldrich, Maybridge, Derwent World Drug Index, NCI and/or Chapman & Hall, can also be searched. The following computer programs can be used for such a search: in particular the program “Unity”, “FLEX-X” (Rarey et al., J. Mol. Biol. 261, 470-489, 1996), “Cscore” (Jones et al., J. Mol. Biol. 245, 43, 1995) from the Sybyl Base environment of the Tripos program package.

A method according to the invention for carrying out the computer-assisted identification of potential ligands is described in more detail below. First, the desired binding region of a ligand in a crystal form according to the invention must be defined. Depending on the desired effect of the ligand, it may be an activator or inhibitor which binds to a regulatory region or a ligand for the active center, then typically having inhibitory properties. The binding region is characterized by appropriate parameters, for example interatomic distances, hydrogen bridge bonding potentials, hydrophobic regions and/or charges, and boundary conditions for the chemical, physical and/or geometric properties of the ligand are defined on this basis. Preferably, the binding region is a region in the acidic loop in the domain III of a calpain or a bond of a ligand to or into the cleft between subdomain IIa and subdomain IIb. Very particularly preferably, at least one of the amino acids already specified above, in particular having the above-mentioned side chains thereof, are involved in the bonding. For a present method according to the invention for identifying compounds, the preceding disclosure on the subject of the invention “compound” as claimed in any of claims 24 to 34 is therefore also hereby incorporated in its entirety. Computer programs then identify, in appropriate databases, those compounds which fulfill the conditions introduced above. Here, it is particularly preferable to use the program package Sybyl Base (Tripos, 1699 South Hanley Road, St. Louis, Mo. USA). It is particularly preferable if the database to be searched provides compounds with information about their respective three-dimensional structures. If this is not the case, a computer program which, before checking whether the specified boundary conditions are fulfilled by a ligand, first calculates its three-dimensional structure (e.g. the program “CONCORD” from the Sybyl environment of Tripos Inc.) is used for a method according to the invention, preferably in a method step (c1).

Typically, the interaction potential between a compound identified, for example in an automated search for a compound in a computer database, and the desired binding region in a crystal form is determined in a method step (d1). A method according to the invention is very particularly preferred when it serves for identifying compounds which are to be docked with a crystal form having the structural coordinates of FIG. 10. The strength of the interaction, determined according to method step (d1), between a compound in a computer database and a crystal form according to the invention provides information about its suitability for being used as a ligand.

A nonautomated method for identifying suitable compounds having ligand character is as follows. A skeleton compound as a starting point for the identification is manually inserted into the space to be filled via the compound to be identified, in the interior or at the surface of the crystal form, for example into the catalytic center of a crystal form according to the invention. For the space still remaining after insertion of the skeleton structure, a search is made for fragments which can interact with the surrounding crystal form and can undergo addition to the skeleton structure. This search for suitable fragments is thus effected in accordance with the geometric and/or physiochemical characteristics of the three-dimensional structure. The search for suitable fragments can be carried out, for example, as an automated computer search with specification of appropriate boundary conditions. Any fragments determined by the operator and/or by the computer search are graphically added to the initial skeleton structure of the starting model in accordance with chemical laws and, after each such step, the interaction potential with the target structure region in the crystal form is calculated. The procedure is continued until the interaction potential between the compound to be identified and the target structure region has been optimized.

The procedure of steps (c), (c1), (d) and (d1) can be repeated cyclically until a compound or a class of compounds has been optimized with respect to its binding behavior, calculated according to an interaction potential which is an algorithm forming the basis of the respective computer program. The large number of potential compounds capable of binding and initially obtained by relatively coarse characterization of the binding region of the crystal form can be increasingly reduced by further specifications of physiochemical or steric characteristics for the desired target compound.

In particular, an obvious combination of the nonautomated and the automated search procedure for suitable compounds is also possible for this purpose. Thus, for example, a compound initially identified by automated computer searching in computer databases could be improved by a nonautomated procedure by addition of fragments having suitable functional groups.

Finally, it is preferable in the present invention to synthesize the compounds obtained by automated computer searching by such methods according to the invention or, if already synthesized and available, to take them from a chemical library and to investigate them in a suitable biological test system for their biological activity. Depending on the result of the biological test system, which may be, for example, a ligand binding assay or an enzyme activity test, further chemical modification may be made to the previously determined compound or the class of compounds. In particular, the use of program packages for identifying suitable fragments, which could be exchanged for fragments present on the previously identified compound or added to said compound may then prove expedient here.

The present invention furthermore relates to methods for identifying a compound having the property of being able to act as substrate, pseudosubstrate, activator or inhibitor, i.e. as a ligand of calpain, the biological test system on the basis of which the so-called screening for suitable target compounds is carried out being introduced at the beginning in a method step (a) in such a method according to the invention. Here too, a binding assay or an enzyme activity test may serve as a biological test system. In the further method steps, those compounds (for example from a library of chemical compounds) which have given a positive result in the biological test are first identified according to (b). These compounds, for example inhibitory or activating ones, are characterized with respect to, for example, their geometric and/or chemical properties, in particular with respect to their three-dimensional structure (method step (c)). If the three-dimensional structure of the compounds determined as hits in the biological test is not known a priori, said structure can be determined by structure elucidation methods, namely X-ray crystallography and/or NMR spectroscopy, or by modeling or, for example, semi-quantum chemical calculations. The compounds obtained in the course of method steps (b) and (c) are then introduced, according to (e), into the atomic structural coordinates of a crystal form according to the invention which are represented as a three-dimensional structure according to method step (d). These may be compounds which bind to the active center or to a segment, relevant for regulation of the active center, in the crystal form. The introduction of a compound into the crystal form can be effected manually according to the operator's experience or in an automated manner by determining a position of the ligand with the strongest possible interaction between the ligand and the target structure region with the aid of appropriate computer programs (“Dock”, Kuntz et al., 1982, J. Mol. Biol. 161, 269-288, Sybyl/Base “FLEX-X”, cf. citation loc. cit.) (method step (e1)).

By representing a compound obtained in this manner graphically in combination with the structure present in the crystal form, it is possible to carry out further method steps which improve the activity of the target compound. In particular, a compound already identified in this manner as being suitable can serve as a template for compounds having even greater activity, for example compounds having an even higher bonding constant. In this context, the methods and approaches already described according to claims 35 to 40 can be used. A preferred procedure is one which is cyclic in that, after the screening in the biological test system, a structural plot is performed and, with the aid of computer methods based on the results obtained in the biological test system, compounds having higher activity are determined, which finally serve in turn as a starting point for the next cycle, which begins with a biological test system. Biological test systems (in vitro or in vivo) can provide information about the quality of the compound, for example as an inhibitor of the biological reaction, i.e., for example, as an inhibitor of the protease reaction, or about the bonding constant, the toxicity or the metabolization properties or possibly about the membrane permeation power of the compound, etc.

Finally, the present invention claims all those compounds which are obtained as a result of a method as claimed in any of claims 35 to 42.

The present invention furthermore relates to processes for the preparation of spatial or crystal forms comprising at least one polypeptide as claimed in any of claims 1 to 23, wherein, in a process step (a), the polypeptide is first overexpressed in an expression system, synthesized or isolated, (b) the polypeptide obtained according to (a) is dissolved in a suitable buffer system and (c) the crystallization is initiated by, for example, vapor diffusion methods. Typically, a concentrated or highly concentrated solution of the polypeptide or polypeptides will be present according to process step (b). If the crystallization of the at least one polypeptide is effected to give crystals according to the invention which have the crystal forms according to the invention, with the aim of using the crystals subsequently for the X-ray structure analysis, the crystallization is followed by the collection of X-ray diffraction data, the determination of the unit cell constants and of the symmetry and the calculation of the electron density maps, into which the polypeptide or polypeptides is or are modeled.

The present invention furthermore relates to methods for the three-dimensional representation of a crystal form of unknown structure comprising at least one polypeptide which contains at least one subdomain of a protein from the family consisting of calpains, which subdomain participates in the catalysis. In such a method, the crystal form having an unknown structure is determined on the basis of a crystal form according to the invention and having a known structure, for example on the basis of the structural coordinates recorded in FIG. 10. There are various possibilities for using known structural coordinates of crystal forms according to the invention for elucidating the structure of polypeptides or polypeptide complexes having 3D structures unknown to date (target structures), which however exhibit certain homologies with the known crystal form according to the invention.

One possibility in this context is the use of phase information which can be obtained from known starting structural coordinates, for example from the structural coordinates according to FIG. 10. The phase information, which is present or can be calculated in case of a known 3D structure of a crystal form according to the invention, is used for this purpose to solve an unknown structure which preferably differs from the known structure only by insignificant conformational deviations (target structure to which a ligand or a ligand other than that in the starting structure is bound for the first time, or derivatives, for example target structures, which are mutants of the starting structure may be mentioned as examples). For this purpose, the phase information of the total known structure or of a part of the known structure is combined with the intensities of the reflections collected for the crystal form of unknown structure, and an electron density map for the crystal form of unknown structure is calculated from this combination. This method is referred to as molecular replacement. The molecular replacement is preferably carried out using the program package X-PLORE (Brünger, Nature 355, 472-475, 1992).

A further possibility for using existing crystal forms according to the invention for elucidating the structure of structurally related sequences or for comparing the primary structures of at least partially homologous peptide chains consists, according to the invention, in (a) comparing the primary sequence of a polypeptide of unknown 3D structure with a primary sequence of a polypeptide which has at least one of the two catalytic subdomains of a calpain (but in particular a calpain) and, in the course of this comparison, identifying homologous segments of the polypeptide of unknown structure and of the primary sequence of a calpain whose spatially or preferably crystal form is known, (b) modeling the homologous segments on the basis of the known 3D structure and finally, according to method step (c), optimizing the modeled 3D structure of the polypeptide with respect to its steric characteristics with the aid of suitable computer programs.

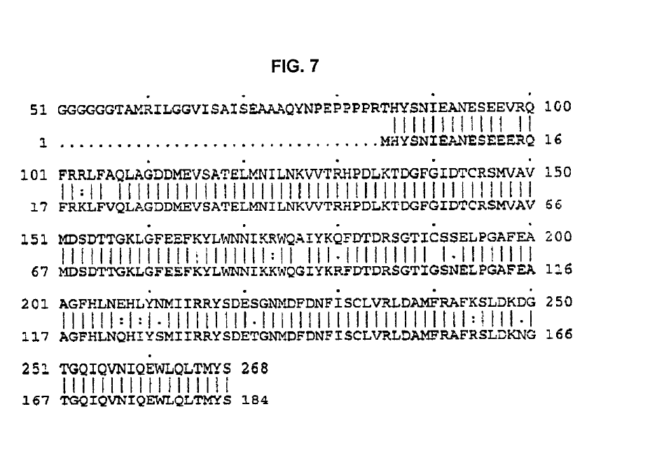

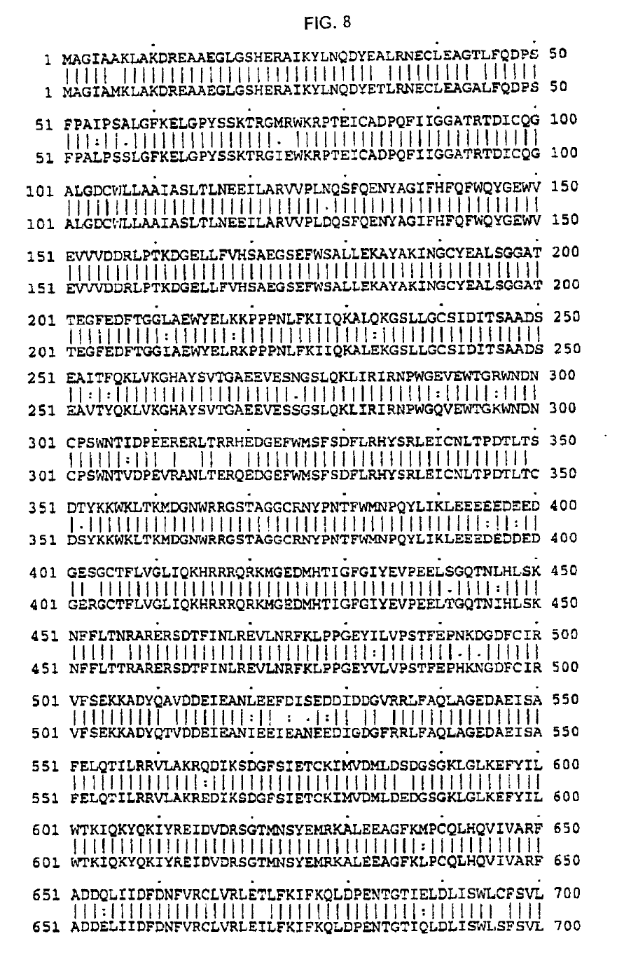

The so-called alignment of the primary sequences of polypeptides of unknown and known 3D structure to be compared according to (a) is a key object of homology modeling. Here, the aligned corresponding amino acids are assigned to different categories, namely positions with identical, similar, remotely similar or dissimilar amino acids. In this context, reference is also made to FIGS. 7 and 8 and to the description of these figures. In the alignment, particular attention must be paid to insertions or deletions between the primary sequences to be compared. The optimization of the target structure modeled on the basis of the known 3D structure, which optimization is performed according to method step (c), can be effected by the molecular dynamics simulation methods or by energy minimization (e.g. Sybyl Base from Tripos, cf. citation loc. cit.).

A method according to the invention for elucidating crystal forms of unknown structure is particularly preferred when the crystal form of known structure is an m- or μ-calpain and the crystal form of an n-calpain is to be elucidated, for example, by molecular replacement or by homology modeling. However, the converse procedure is also possible, for the determination of a μ-calpain structure on the basis of a known m-calpain crystal form, or vice versa. On the basis of a known calpain crystal form, according to the invention, of a host organism, it is also possible to determine the crystal form for a calpain complex of another host organism.

Consequently, structural coordinates of crystal forms according to the invention can serve, through homology modeling, as structural models for sequential homologous polypeptides of unknown 3D structures. In the course of the homology modeling, program packages are used, in particular such a modeling can be carried out using the Insight II modeling package (Molecular Simulations Inc.).

Finally, the present invention also discloses the use of inhibitors and/or activators of the catalytic activity of calpain, in particular of human calpain, very particularly of human m-calpain, as claimed in any of claims 24 to 34 or obtained from a method as claimed in any of claims 35 to 42 for the preparation of a drug, for use as a drug or as an active substance which is contained in a pharmaceutical composition. A calpain activator or inhibitor according to the invention is incorporated in a pharmaceutical composition with at least one further active substance and/or the pharmaceutical composition is incorporated as a drug into a formulation familiar to a person skilled in the art. The formulation is dependent in particular on the route of administration. This may be oral, rectal, intranasal or parenteral, in particular subcutaneous, intravenous or intramuscular. Pharmaceutical compositions which contain such an inhibitor and/or activator may have the dosage form of a powder, of a suspension, of a solution, of a spray, of an emulsion or of a cream.

An inhibitor and/or activator according to the invention can be combined with a pharmaceutically acceptable excipient material having a neutral character (such as, for example, aqueous or nonaqueous solvents, stabilizers, emulsifiers, detergents and/or additives and optionally further colors or flavors). The concentration of an inhibitor and/or an activator according to the invention in a pharmaceutical composition may vary between 0.1% and 100%, depending in particular on the route of administration. A pharmaceutical composition or a drug containing an inhibitor and/or activator according to the invention can serve in particular for the treatment of ischemic conditions, muscular dystrophy and/or tumor diseases.

The present invention is explained in more detail in the following figures, in which

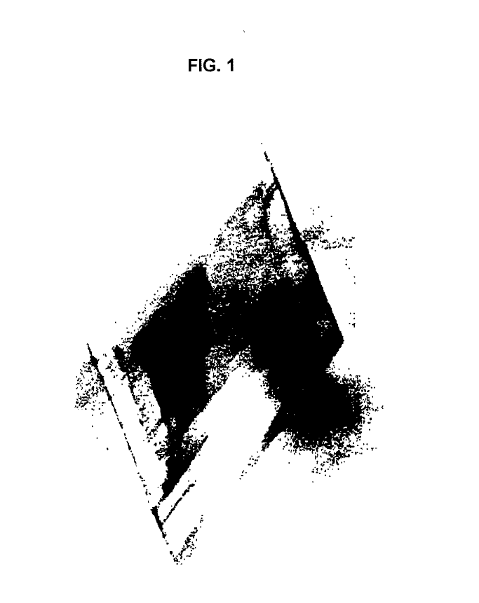

FIG. 1 represents a crystal, according to the invention, of crystal type 1 having a crystal form, according to the invention, of human m-calpain (small and large subunits), with the appearance of a rhombic lamella and the unit cell constants a=64.78 Å, b=133.25 Å, c=77.53 Å and β=102.07°. The crystal shown has a size of about 1 mm×1 mm×0.1 mm.

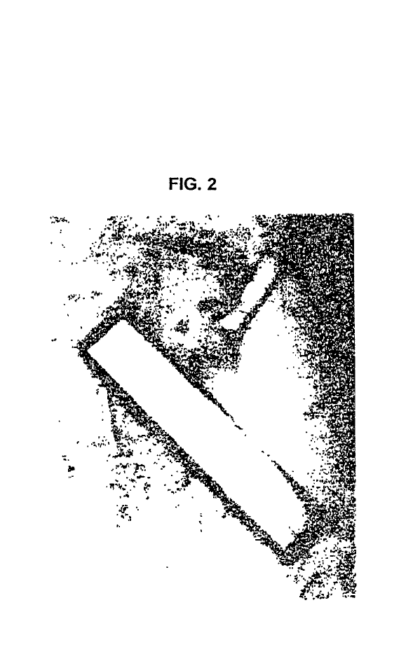

FIG. 2 shows a crystal of crystal type 2, likewise having a crystal form comprising a large and a small subunit of human m-calpain, with lamellar or prismatic morphology. It is characterized by unit cell constants a=51.88 Å, b=169.84 Å, c=64.44 Å and β=95.12°. The crystal shown in FIG. 2 has a size of about 1 mm×0.2 mm×0.1 mm.

FIG. 3 represents the amino acid sequence of the small subunit of m-calpain (30 kDa subunit). The amino acid sequence is stated in a one-letter code.

FIG. 4 represents the amino acid sequence of the large subunit (80 kDa subunit) of human m-calpain. Here too, the amino acid sequence is stated in the one-letter code.

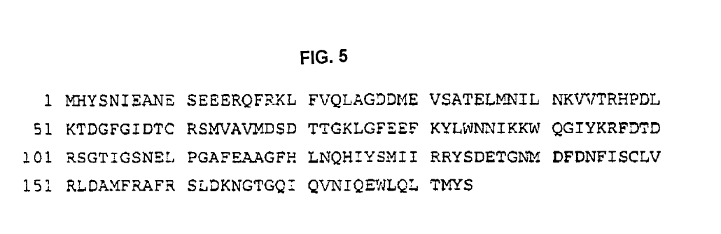

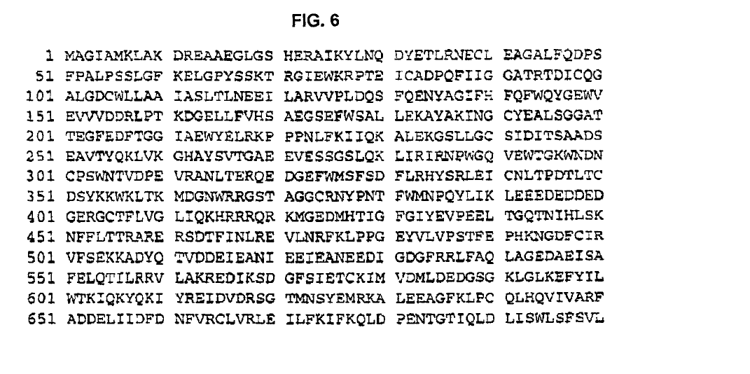

FIG. 5 represents the amino acid sequence (one-letter code) of the small subunit of rat m-calpain (species: Rattus norvegicus)

FIG. 6 shows the amino acid sequence of the large subunit of rat m-calpain (80 kDa subunit) (species: Rattus norvegicus) in the one-letter code.

FIG. 7 shows a comparison of the amino acid sequences between the small subunits of m-calpain of the rat and of the human form. The sequences used for the comparison correspond to the sequences shown in FIG. 3 and 5, the upper row in each case corresponding to the human sequence and the lower row to the rat sequence. If identical amino acids are present in the corresponding positions in each case, they are marked by a line, similar amino acids are marked by double points and only remote similarities of the side chains of corresponding amino acids are marked by a point. In this comparison, a sequence identity of 95.18% and a sequence similarity of 94.57% were obtained. Gaps in one of the two comparison sequences do not occur. There are no identities and/or similarities between amino acids at the following corresponding positions (only the human sequence position is mentioned): V98, A106, A176 and C190.

FIG. 8 shows a comparison of the amino acid sequences of the large subunit of rat and human m-calpain. The upper line in each case corresponds to the human sequence, as also shown in FIG. 4, and the lower line in each case corresponds to the rat sequence, as shown in FIG. 6. The explanations for FIG. 7 are applicable analogously in the present case. Differences between the human and the rat sequence are to be found in the positions (only the position in the human sequence is stated in each case in the following): A6, A34, T54, R74, E311, E313, R314, R317, H319, S350, S403, N456, A511, F523, I525, D531, V534, S586, T671 and C696. The percentage of identical amino acids is 93%, and the percentage of similar amino acids as a result of this comparison is 96.86%. Gaps in one of the two sequences could not be observed in the comparison of sequences.

FIG. 9 forms the amino acid sequences of the large subunit of human m-calpain (80 kDa subunit) and the amino acid sequence of the small subunit of human m-calpain (30 kDa), in each case in the one-letter code, in combination with structural data. The symbols shown under the respective structure assumed in each case by the amino acid characterized in this manner. Here, the cylinders represent amino acids which assume a helical structure and arrows represent those amino acids which are part of strands with a so-called β-conformation. The assignment of the amino acids present in a conformation with a secondary structure is performed as a function of the torsion angles of the main chain about the Cα atom of the respective amino acid, reference being made to textbooks of biochemistry, for example to Lehninger, Nelson & Cox, Prinzipien der Biochemie [Principles of biochemistry], Spektrum Akademischer Verlag GmbH, 1998, for details. To the right of each of the secondary structure symbols, the designation of the respective secondary structure is stated according to clear nomenclature (α for helix, β for a strand in the β-conformation, then consecutive numbering of the secondary structure and finally the domain designation in Roman numerals). Above the amino acid sequence in the one-letter code are black arrows which are oriented in opposite directions and mark the domain boundaries. Functionally important residues are designated by symbols arranged above said residues (for example, amino acids from the catalytic reaction center or further important amino acids from the subdomains IIa and IIb by red and black triangles, respectively, acidic amino acids of the switch loops in the domain III and the neighboring positively charged amino acids of the subdomain IIb by red and blue circles, respectively). The amino acids participating in the Ca binding of the domain VI of the small subunit are characterized by black circles.

According to a subdivision, modified according to Sorimachi et al. (cf. loc. cit.), of the two polypeptide chains forming the calpain complex, and taking into account the structural conditions, the following domain boundaries can be determined for human m-calpain. Large subunit: domain I (M1 to E16), linker region between domain I and domain II (G17 to A92), subdomain IIa (T93 to G209), subdomain IIb (G210 to N342), linker region between domain II and domain III (L343 to K355), domain III (W356 to A511), linker region between domain III and domain IV (V512 to D529), domain IV (I530 to L700). Small subunit: domain V (M1 to E94) and domain VI (S95 to S268).

Below, the individual secondary structures of the large and small subunits are described, beginning at the N terminus of the large subunit.

In the case of the large subunit (80 kDa subunit), a helical structure is present between the amino acids 4 and 15 (helix α1I) in the domain I (in green). Amino acid 17 marks the beginning of the domain II (secondary structures of the domain IIa in yellow), the amino acids 31 to 44 initially being present in an a-helical conformation (α1II). The next secondary structure, likewise an α-helix, begins with amino acid D104 and runs up to amino acid N118 (α2II). It is immediately followed by a further α-helix (α3II), beginning with amino acid E118-V125. From amino acid I138, the large subunit repeatedly assumes the conformation of β-pleated sheets, namely β1II (I138-Q145, E148-D156, P159 to K161 and E164-L166 (β4II)). A helix can be observed between the amino acids F176 and G190 (α4II) and Y192-S196 (α5II). Finally, a helix of amino acid T200-T208 is present as the final secondary structure in the domain IIa.

The now following secondary structures of the domain IIb are characterized in red. G210 defines the start of the domain IIb, a β-pleated sheet (β5II) running from I211 to L217. Then, from amino acid N223, the polypeptide assumes a helical structure up to Q233 (β7II). From L237 to I242, the conformation of a β-strand (β6II) is present. Other secondary structures in the domain IIb are two β-pleated sheets from H262 to S274 and S277 to N286. This is followed, in the domain IIb, by the following secondary structural elements: α-helix from P309 to T316, a β-pleated sheet from G322 to M326, an α-helix from S327 to R333 and finally a β-pleated sheet from S336 to N342.

For the domain III, the following secondary structures are emphasized by blue marking: a β-pleated sheet from K357 to W365 (β1III), a β-pleated sheet from Q386 to L391 (β₂III), a β-pleated sheet from C405 to K414 (β₃III), a further β-pleated sheet from T428 to E435 (β₄III), an α-helix from S449 to N456 (α₁III), and finally four β-pleated sheets from R469 to L477 (β₅III), G480 to F489 (β₆III), G495 to E504 (β₇III) and finally D508 to V512 (β₈III).

The domain IV, a calcium-binding domain, has exclusively α-helical secondary structures. In the corresponding sequence, the following α-helices may be mentioned (marked yellow): I530 to A542 (α₁IV), S549 to L561 (α₂IV), E575 to D585 (α₃IV), G593 to V616 (α₄IV), N623 to G535 (α₅IV), P639 to F650 (α₆IV), D658 to D680 (α₇IV), and finally D690 to L700 (α₈IV)

In the small subunit, the first 84 amino acids from the domain V are not defined in the electron density map owing to considerable flexibility, which is why the corresponding structural data are not available. The domain VI, starting with S95, which likewise represents a calcium-binding domain, also contains exclusively α-helical structural elements. These are specifically the following α-helical sequence segments: S95 to L108 (α₁VI), S116 to R130 (α₂VI), G140 to D152 (α₃VI), G160 to D182 (α₄VI), C190 to G202 (α₅VI), L209 to S218 (α₆VI), D225 to D247 (α₇VI) and finally N257 to Y267 (α₈VI).

FIG. 10 lists the coordinates of the individual atoms of the two subunits of human m-calpain. The coordinates reproduce the structure of the two subunits in an x, y and z coordinate system. The sequence of the atoms in FIG. 10 is defined by their association with amino acids of are listed from the N to the C terminus initially for the large and then for the small subunit of human m-calpain. Since the amino acid methionine M1 present in position 1 of the N terminal of the long subunit does not give rise to any electron density in the electron density map, owing to strong conformational flexibility typically observed at the termini, the amino acid A2 is listed at the beginning in FIG. 10. FIG. 10 shows, in the internationally customary nomenclature (Bernstein et al., J. Mol. Biol. 112, 535 et seq., 1977, including the publications cited there), the structural coordinates of all atoms of a crystal form according to the invention (as part of a crystal), of human m-calpain, apart from hydrogen atoms, which do not manifest themselves in the electron density map through corresponding electron densities. The positions of the oxygen atoms of the water molecules determined for the crystal form are also contained in FIG. 10.

FIG. 11 shows a schematic representation of the three-dimensional structure of the two subunits of human m-calpain in a form familiar to a person skilled in the art, the structural conformation of the complex in the absence of calcium being shown. This is a so-called ribbon representation which takes into account only the backbone of the polypeptide chain and does not reproduce the side chains located in each case on the Cα atom or their conformation. The positions of the backbone of the two polypeptide chains with the free torsion angles about the Cα atoms of each amino acid of human m-calpain plotted in the electron density map are shown by the path of the ribbon in the ribbon diagram, said torsion angles determining the structure. In this method of representation, helical structures are marked as helices and β-pleated sheets as arrows, while sequence regions without such secondary structural elements are shown as filaments. In the present case, the individual domains of the small and large subunit are marked in color. The secondary structural units shown in each case furthermore correspond to the structural data which are assigned in FIG. 9 to the primary sequence of the two polypeptides.

The m-calpain complex has the form of a flat oval disk. The upper and lower poles (according to the reference orientation of FIG. 11) are formed by the two catalytic subdomains IIa and IIb or by the calmodulin-like domain pair from the large and small subunits, respectively. The domain III and the N-terminal domains I (large subunit) and V (small subunit) link the two calmodulin-like domains to the two catalytic subdomains. The similarity of the crystal forms of m-calpain to be observed in general in the X-ray structure analysis for each of the two crystal types (1) and (2) indicates that the structure of calcium-free m-calpain, as shown in FIG. 11, is independent of the specific packing in the crystal.

The amino acid chain of the large subunit begins with the so-called anchor helix (α1I) of the domain I (in green), which is positioned in a semicircular cavity of the domain VI. From there, the amino acid chain runs straight to the domain IIa. Owing to its interactions with the domain VI, the anchor helix results, inter alia, in the two subunits of the m-calpain complex being held together. In the case of the amino acid Gly19 of the large subunit (for method of counting for human m-calpain, cf. FIG. 4), the amino acid chain of the large subunit makes contact with the subdomain IIa (in yellow), the polypeptide chain folding up in this region to give an α-helix (α1II) and various turn structures with formation of an outer polar surface of the complex.

From the amino acid T93, the amino acids of the long polypeptide chain are involved in the structural region of the catalytic domain, which is topologically related to the catalytic domain of the protease papain. However, there are considerable differences compared with the catalytic domain of papain by virtue of the fact that the two subdomains characteristically separated in the calpain differ considerably, with respect to both the length and their conformation, from the corresponding structure in the case of the protease papain.

From the perspective chosen in FIG. 11 (reference position of the complex), the domain IIa forms the left half of the catalytic cleft, which is composed of amino acids of both subdomains in the space between the subdomains IIa and IIb (in red). In FIG. 11, the amino acids essentially participating in the catalytic, i.e. proteolytic, reaction are shown with the position of their side chains, but without showing the hydrogen atoms. These are the amino acids Cys105 and Trp106 of the subdomain IIa and His262, Asn286, Trp288 and Pro287 of the subdomain IIb.

At the conformational “hinge” between Gly209 and Gly210 of the large subunit, the polypeptide chain passes over into the subdomain IIb having a drum-like structure, where it forms a typical 6-strand β-pleated sheet (strands β₅II to β₁₀II) The drum-like structure is achieved by the supersecondary structure, i.e. the arrangement of the secondary structural elements. Particularly noteworthy is the sequence of the amino acids Asn286, Pro287 and Trp288 with their particular conformations, Asn286 participating in the catalytic reaction. From the strand β₁₀II, the polypeptide chain runs initially toward subdomain IIa before it becomes, via an open loop structure (a so-called linker region), part of the domain III (in blue).

The domain III essentially consists of two opposite β-pleated sheets, each β-pleated sheet having four antiparallel strands. This leads to a compact tertiary structure which has a β-sandwich form. The topology of this domain is slightly reminiscent of the TNFα monomer or some virus surface proteins. The basic amino acids His415 to His427 in the domain III, which form a loop lying in the center of the calpain complex, are noteworthy. Also striking is the negatively charged β₂III/β₃III loop which is exposed to solvent and has ten acidic amino acids within the segment Glu392 to Glu402 comprising eleven amino acids. This loop has been well determined crystallographically. It is arranged spatially close to the helix α₇II of the subdomain IIb and the open loop of the subdomain IIb and interacts electrostatically with the numerous positive charges of these two segments of the subdomain IIb.

From the domain III, the amino acid chain runs along the calmodulin-like domain IV in an extended conformation, with the result that a plurality of acidic amino acids are in direct contact with the solvent. This is a further, long linker region (in magenta) without characteristic secondary structure, which extends to the lowermost position of the domain IV (in yellow) according to the reference perspective chosen here. The tertiary structure of the domain IV begins at the amino acid Ile530 and is substantially known from the folding of the structure of the isolated domains VI of rat calpain and of porcine calpain, which structures are known from the prior art. Both domains IV and VI show similarities to other calcium-binding domains, namely “calmodulin” domains, with EF hand motif. The domain IV (in yellow) has, as secondary structural elements, eight α-helices which are linked by characteristic linker regions, with the result that five of the well known supersecondary structural elements, which are designated as EF hands, are formed (Tooze & Brandén, Introduction into Protein Structure, Garland Publishing Inc., December 1998, 2nd Edition).

The N-terminal part of the polypeptide, which forms the small calpain subunit, is rich in glycine residue and shows no electron density usable for structure determination. From amino acid Thr85 of the domain V, a three-dimensional structure can be assigned to the small subunit (according to FIG. 11 in red), but the polypeptide chain is not present here in one of the two typical secondary structures. At that surface of the crystal form which is exposed to solvent, the polypeptide chain folds back and, with the α-helix a1VI, reveals there the first secondary structural element of the domain VI (in orange).

The domain VI likewise has (like domain IV), five EF hand motifs and, together with the domain IV of the large subunit, forms a quasi-symmetrical heterodimer, since the domain VI is in the structural vicinity of the domain IV, namely on the left of the perspective of the small subunit chosen in FIG. 11. Here, the helices α₆VI, α₇VI and α₈VI and the linker region between α₇VI and α₈VI (α₇VItα₈VI) are involved in the symmetrical interdimer contacts.

The two domains IV and VI are not involved primarily in the regulation of the catalytic activity of calpain since the binding of calcium ions does not give rise to any structural changes, as is evident from a comparison with the crystal forms of the domain VI of rat calpain with bound calcium, which crystal forms are known from the prior art. The calmodulin-like domains can therefore perform primarily structural functions. The present investigations also indicate that the calcium binding to the domains IV and VI leads to dissociation of the two subunits of a calpain, in particular of an m-calpain.

The catalytic domain is formed by the two subdomains IIa and IIb. In the calcium-free crystal form, as shown in FIG. 11, no catalytically active conformation is present; rather, a clear cleft is evident between the two above-mentioned subdomains. In the three-dimensional structure shown in FIG. 11, the two catalytically active side chains of Cys105 (SG) and ND1 of His262 are 8.5° apart, but, for the catalysis, the imidazole side chain of His262 must be brought into the vicinity of Cys105 and at the same time the hydrogen bridge bond must be formed between His262 NE2 and Asn286 ND2. This cleft between the subdomains can be closed if the subdomain IIb is rotated 50° and translated 12 Å toward the subdomain IIa. Only after such a movement can calpain display its catalytic activity. In the course of the conformational activation of calpain, the indole group of Trp288 of the large subunit can finally also exercise its corresponding protective function with respect to hydrogen molecules and thus ensure an undisturbed proteolysis of the substrate.

Physiologically, the proteolytic activity of calpain is ensured only in the presence of calcium. As already mentioned above, the binding of calcium to the domains IV and VI of the calpain complex has no regulatory effect on the structure of the catalytic domain. Rather, addition of calcium ions at the acidic residues of the acidic loop of the domain III appears to be a decisive calcium binding site for a combination of the subdomains IIa and IIb for the formation of an active reaction center. This gives rise to change of conformation, which then also has regulatory effects.

The acidic loop (β₂IIItβ₃III) has, as already mentioned above, ten negatively charged side chains which point away from one another owing to electrostatic repulsion. This acidic loop is in direct contact with the amphipathic α₇II-helix of the subdomain IIb and the open loop of the subdomain IIb, with the lysine residues K226, K230, K234, K354, K355 and K357. Direct salt bridges beyond the domain boundaries form between some of the above-mentioned acidic and basic amino acid side chains, since the negative and positive electrostatic potentials carried by the corresponding side chains attract one another. The binding of one or more calcium ions, for example under corresponding physiological conditions which, inter alia, increase the calpain activity after Ca liberation, to this acidic loop ensures at least partial charge compensation. Preferably, the acidic amino acids of the acidic loop bind more than one calcium ion, for example two or three calcium ions.

The binding of at least one calcium ion at this site compensates the extremely great accumulation of negative charges in this structural region and simultaneously also leads to more compact folding in the region of the acidic loop since the electrostatic repulsion of the negatively charged side chains in this structural region is reduced. A reduction of electrostatic interaction between the acidic loop and the above-mentioned basic amino acids also makes it possible for the subdomain IIb to become detached from its fixed position shown in FIG. 11 and to move toward the subdomain IIa, with the result that the cleft present between the subdomains in the absence of calcium (as shown in FIG. 11), which simulates the inactive state of the protease, is closed.

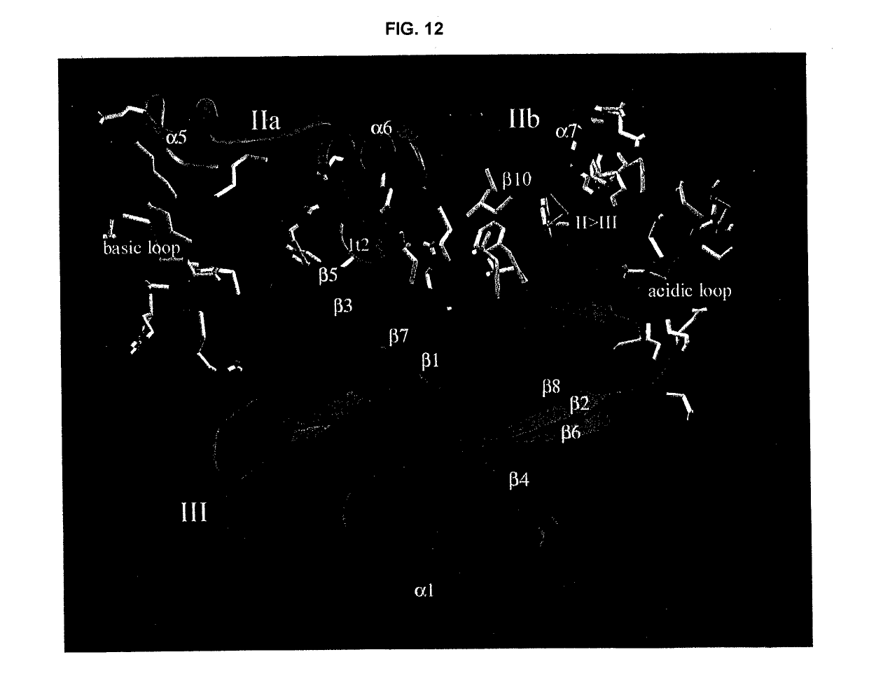

A conformational change of the subdomain IIb, which is triggered in this manner and converts the complex from the inactive to the active form, is also facilitated by the hydrophobic region, shown in FIG. 12, between the β-strands β₅II and β₁₀II of the β-pleated sheet of the domain IIb and the strands β₃III, β₅III and β₇III of the β-pleated sheet of the domain III. The collection of the hydrophobic amino acid side chains in this region permits a sliding movement of the subdomain IIb toward the subdomain IIa. At the same time, as a result of its numerous polar interactions with the domain III, the subdomain IIa is positioned in a defined manner relative to said domain III and is thus fixed.

While the m-calpains have ten negative charges in the region of the acidic loop, only eight negative charges are arranged in the corresponding structural region in the case of μ-calpains, as a result of the primary structure. Consequently, m-calpains require a stronger charge compensation in this region than μ-calpains, which typically require lower calcium levels or no calcium for the conformational change and the activation of the catalytic region. Incidentally, a corresponding pattern is observed in the neighboring basic region of μ-calpains. Basic amino acids at the positions 226 and 357 of the large subunit occur only in the case of m-calpains, so that furthermore a smaller number of structurally adjacent basic amino acids is opposite a smaller number of acidic amino acids in the acidic loop in the case of the μ-calpains.

Finally, further compounds can reduce the interaction between the acidic loop of the domain III and the basic amino acids of the subdomain IIb in the case of m- and/or μ-calpains. Thus, preferably acidic phospholipids, such as, for example, phosphorylated phosphatidylinositols, can lower the calcium concentrations required for the activation, by interacting with the basic amino acids of the subdomain IIb and thus reducing the intensity of interaction between the basic and acidic amino acids. Thus, in particular acidic phospholipids, as ligands on a spatial and/or crystal form claimed here, are activating ligands preferred according to the invention. In another preferred case, ligands according to the invention have positive charges and/or partial charges which compensate the negative charges of the acidic loop and thus release the subdomain IIb for the calpain-activating conformational change.

FIG. 12, too, shows a schematic “ribbon” representation of a crystal form according to the invention, with its three-dimensional structure, focused on a section of the large subunit of human m-calpain in the absence of Ca ions. The domain III (in blue) is distinguished by μ-pleated sheet structures, each having oppositely oriented μ-pleated sheets, the present illustration being focused in particular on the contact region of the domain III with the structural elements of the subdomains IIa and IIb. Shown in yellow in FIG. 12 are therefore parts of the helices α₅II and α₆II, and the helix α₇II of the domain IIb (in red) with the positioning of side chains of selected amino acids. In particular, the arrangement of the acidic amino acids in the so-called acidic loop of the domain III is emphasized. As is clearly evident in FIG. 12, this charge is at least partly compensated by positive charges and/or partial charges of side chains of the amino acids arranged in the helix α₇II. Those amino acid side chains with corresponding conformation which are involved in hydrophobic or polar interactions in the boundary region between the subdomains IIa and IIb or the domain III are shown in the middle of FIG. 12. On the left of FIG. 12 (likewise corresponding to the reference position of FIG. 11), the character of the basic loop of the domain III is represented by the image of the basic amino acids.

The present invention is explained in more detail by the following embodiment.

1. Overexpression, Purification and Characterization of Human m-calpain

Full-length human m-calpain which has the sequence Gly-Arg-Arg-Asp-Arg-Ser at the N terminus of the large subunit, followed by the natural sequence beginning with Met1, was expressed in a baculovirus expression system and purified in a manner corresponding to the prior art and as described in detail by Masumoto et al. (J. Biochem. 124, 957 961, 1998). The content of the publication cited above is in its entirety also part of the disclosure of the present invention. In particular, the information disclosed by Masumoto et al. with respect to the materials, to the preparation of the baculovirus transfer vector of human calpain of the large or of the small subunit, to the cell cultures, to the preparation of the recombinant virus, to the expression of human m-calpain and to the purification of the recombinant human m-calpain and to all further experimental procedures for the overexpression, purification and characterization of human m-calpain is a part of the description of the present invention.

2. Protein Crystallization

Before the crystallization, the protein was concentrated to a concentration of approximately 14 mg/ml, and the buffer was changed for 10 mM Tris/HCl pH 7.5, 50 mM NaCl, 1 mM EDTA and 1 mM DTE. The protein concentration was determined by absorption spectroscopy using a molar extinction coefficient of A=17.2 (10 mg⁻¹ ml⁻¹ cm⁻¹) at 280 nm. With 20% of polyethylene glycol (PEG) 10000, 0.1 M Hepes/NaOH (pH 7.5), small crystals having a longest dimension of not more than 100 μm were observed. However, such crystal growth was obtained only in 5 to 10% of the experiments even under these conditions. By adding isopropanol and guanidinium chloride, it was possible to improve the crystal size and the crystal growth. Nevertheless, crystals which have a size suitable for X-ray structure analysis could be obtained only by so-called “macroseeding” of the small crystals using the technique of suspended and stationary drops with corresponding vapor diffusion methods. Drops of 6.6 μl, consisting of 4 μl of the protein solution, 2 μl of the precipitation solution (100 mM Hepes/NaOH pH 7.5, 15% of PEG 10000, 2.2% of isopropanol) and 0.6 μl of 1 M guanidinium chloride, were brought into equilibrium against 400 μl of the precipitation solution at 200° C.

Two different crystal types were obtained, both having the space group P2₁.

3. X-Ray Diffraction

The crystals grown were unsuitable for exposure to X-rays at room temperature. A corresponding cryo-protection buffer was therefore used. For this purpose, the crystals were removed from the drop with the aid of a loop, referred to as a cryo-loop, and transferred to 20 μl of the reservoir buffer. 10 μl of 88% strength glycerol were slowly added in order to accustomize the crystal to the cryo-buffer conditions. The equilibrated crystals were placed in tubes and cooled abruptly with a gas flow from a cryostat containing liquid nitrogen (Oxford Cryo Systems). The X-ray diffraction patterns were collected at 100 K on an MARCCD detector at a BW6 “Beamline” of the German electron synchrotron in Hamburg (DESY) using monochromatic X-rays by standard methods. A corresponding description of the standard method is to be found in Helliwell (Macromolecular Crystallography with Synchrotron Radiation, Cambridge University Press, Cambridge 1992), which in its entirety is part of the present disclosure.

4. Determination of the Unit Cell Constants and of the Resolution

For the two crystal types obtained in the crystallization, a divergent resolution was determined. While reflections up to a resolution of 2.8 Å were observable for crystal type 1, a resolution of max. 2.1 Å was obtained for crystal type II. After collection of the data of the diffraction pattern it was possible to determine the cell constants of the unit cell, namely the dimensions of the unit cell and the angles in the unit cell, and the orientation of the crystals. The symmetry of the unit cell was also determined therefrom.

(a) Crystal Type 1

Crystal type 1 is macroscopically a rhombic lamella and has the cell constants a=64.78 Å, b=133.25 Å, c=77.53 Å and β=102.07°. These crystals have the monoclinic space group P2₁. The crystals grow to a maximum size of 1.0 mm×1.0 mm×0.1 mm. A 3.0 Å data set comprising over 360 positions (exposure time 50 sec in each case) was collected. The Vₘ value is 3.4 Å³/Da. For a more detailed explanation of this information, reference is made to Matthews (J. Mol. Biol. 33, 491-497, 1968). There is one heterodimer in the asymmetric unit, corresponding to a solvent fraction of 61% by volume. Altogether, 223,726 reflections were recorded, which in turn corresponds to 25,010 unique reflections (R_{merge}=5.6%). It is possible to calculate that 96.9% of the theoretically possible unique reflections were collected. Further data on crystal type 1 are shown in Table 1.

(b) Crystal Type 2

Crystal type 2 has a lamella to prism-like appearance with unit cell constants of a=51.88 Å, b=169.84 Å, c=64.44 Å and=95.12°. The crystals grow to a maximum size of 1.0 mm×0.2 mm×0.1 mm and have a resolution of up to 2.1 Å. Further data on crystal type 2 are shown in Table 1 and follow under 5.

5. Obtaining the Phase Information

Since, in order to calculate electron density maps, structure factors with amplitude and phase information must be used, but the diffraction pattern provides only the amplitude information on the basis of the intensity measured for each reflection, it is necessary to use methods for determining the phase information. In the present case, heavy metal atom derivatives were used for this purpose, and the multiple anomalous dispersion method (MAD: Hendrickson & Latman, Acta Crystallographica B26, 136-143, 1970; Hendrickson & Teeter, Nature 290, 107-113, 1981; Bijvoet, Nature 173, 888-891, 1954) was used. In the MAD measurements, data for the gold derivative crystal were collected at wavelengths of λ=1.0092 Å (absorption edge of gold) and λ=1.004 Å) and at the more remote wavelength of λ=1.100 Å. For the mercury derivative crystal too, three MAD measurements were performed at different incident wavelengths (λ=1.0399 Å (absorption edge of mercury) and λ=1.0392 Å) and at the more remote wavelength of λ=1.100 Å. In the present case, the phase information from the measurements for the mercury derivative was combined with the amplitude information of the gold derivative to calculate an electron density map for the crystal form.

In order to obtain crystals which have crystal forms with intercalated heavy metal ions, gold and mercury derivatives were prepared by so-called “soaking” of natural crystals with 5 mM gold triethylphosphine and with phenylmercury.

(a) Gold Derivative

Crystals having crystal forms with intercalated gold derivative scattered up to a resolution of 2.3 Å. A corresponding data set with 330 positions (exposure time 50 sec per position) was collected. On the basis of the Vₘ value of 2.61 Å³/Da, one heterodimer per asymmetric unit was determined in crystals, which in turn corresponds to a solvent fraction of 53% by volume in the crystal. Altogether, 400,860 reflections were recorded and these were combined to give 47,236 unique reflections (R_{merge}=4.5%), which corresponds to 94.8% of the theoretically possible reflections (Table 1). Five gold positions were identified in the anomalous Patterson difference maps. The positions for the gold derivative as well as for the mercury derivative were refined using the program MLPHARE from the CCP4 program package (cf. citation, loc. cit.).

(b) The Phenylmercury Derivative

In the case of this derivative, reflections up to 2.4 Å were collected. For the phenylmercury derivative, using DENZO (Otwinowski and Minor, 1993, DENZO: Film Processing for Macromolecular Crystallography, Yale University, New Haven, Conn.), 421,730 reflections were identified, scaled and combined to give 48,363 unique reflections (R_{merge}=4.7%), i.e. 93% of all possible unique reflections. Here, three positions were determined for mercury in the anomalous Patterson difference maps. In addition, a reduction was performed using SCALEPACK (Otwinowski and Minor, 1993, see above). The data were further processed using the corresponding programs of the CCP4 program sequence (Collaborative Computational Project No. 4, 1994, Acta Crystallog., Sec. D50, 760-763).

6. Building of the Structural Model:

To build the structural model, the structural factor amplitudes of the gold derivative were provided with the phases of the phenylmercury derivative, making it possible to calculate for the gold derivative an electron density map which showed good contours up to a resolution of 2.4 Å. The method according to La Fortelle and Bricogne [Methods Enzymol. 276, 472-494 (1997), (SHARP)] was used. In this way, it was possible to model 90% of the amino acids of the crystallized protein into the electron density map. Here, the first 84 amino acids of the small subunit are not taken into account since it was not possible to observe sufficient electron density. The modeling of the two amino acid chains, i.e. polypeptides, of the large and of the small subunit into the electron density map was performed on a Silicon Graphics workstation (Indigo) with the aid of the TurboFRODO software package (Roussel and Cambileau, Silicon Graphics, Mountainview, Calif., USA (1998)).

7. Refinement of the Structural Model

This partial model produced was refined crystallographically. For this purpose, the protein model was completed with the aid of an improved combined-phase Fourier transformation. The software packages REFMAC from the CCP4 program series and X-PLORE (Brünger, X-PLORE Version 3.1, A System for X-Ray Crystallography and NMR Spectroscopy, Yale University Press, New Haven, Conn. (1993)) were used for calculating the electron density maps and for carrying out the crystallographic refinement. Finally, to complete the crystallographic model, water molecules were inserted into the electron density map and the individual atomic temperature factors were refined. In the final model, an R factor of 20.6% (free R factor=26.6%) was achieved for 38,544 reflections. In this final model, the amino acids Ala2-Leu700 of the large subunit and the amino acids Thr85-Ser268 of the small subunit were well defined. The final model had 7097 atoms (except for hydrogen atoms), 5 gold ions and 352 water molecules. The final standard (rms) deviations of the corresponding standard bond lengths and lengths and standard angles were 0.0007 Å and 1.179°. 85% of all main-chain torsion angles are in the allowed or extended allowed ranges of the Ranachandran plot.

TABLE 1

| | | Crystal type II |
|---|---|---|
| Constant | Crystal type I | (Gold derivative) |
| Space group | P2₁ | P2₁ |
| Crystal morphology | Lamellae | Rod/rectangular |
| | | cube |
| Unit cell constants | a = 64.78 Å | a = 51.88 Å |
| | b = 133.25 Å | b = 169.84 Å |
| | c = 77.53 Å | c = 64.44 Å |
| | β = 102.07° | β = 95.12° |
| Vₘ(ÅDa⁻¹) | 3.14 | 2.61 |
| Heterodimer per | 1 | 1 |
| asymmetric unit | | |
| Estimated solvent | 61 | 53 |
| fraction (%) | | |
| Diffraction limit | 3.0 | 2.3 |
| (Å) | | |
| Rotational angle | 0.5 | 0.4 |
| between the exposure | | |
| positions | | |
| Exposure time per | 50 s | 50 s |
| position | | |
| Total rotation of | 180° | 132° |
| the crystal during | | |
| data collection | | |
| Number of | 223,726 | 400,680 |
| reflections measured | | |
| Number of unique | 25,010 | 47,236 |
| reflections | | |
| R_{merge}^{a} | 0.056 | 0.045 |
| Completeness of the | 96.9% | 94.8% |
| data set | | |
| Completeness of the | 92.5% | 92.2% |
| data set in the | | |
| outermost spherical | | |
| cap of inverse space | | |

1. A spatial form of at least one polypeptide, wherein at least one polypeptide in the spatial form contains at least one (sub)domain of a protein from the family consisting of the neutral Ca-activated cysteine proteinases (calpains), which (sub)domain participates in the catalysis.

2. The spatial form of at least one polypeptide as claimed in claim 1, wherein the neutral Ca-activated cysteine proteinase is selected from the group consisting of isozymes from the family of the ubiquitously expressed calpains and of isozymes from the family of the calpains expressed in a tissue-specific manner (n-calpains).

3. The spatial form of at least one polypeptide as claimed in claim 1 or 2, wherein the neutral Ca-activated cysteine proteinase is an isozyme from the group consisting of m- or μ-calpains.

4. The spatial form of at least one polypeptide as claimed in any of the above-mentioned claims, wherein the calpain is of human origin.

5. The spatial form of at least one polypeptide as claimed in any of the above-mentioned claims, wherein at least one polypeptide of the spatial form contains the amino acid sequence of the subdomain IIa and/or the amino acid sequence of the subdomain IIb of an m-calpain.

6. The spatial form of at least one polypeptide as claimed in any of the above-mentioned claims, wherein at least one polypeptide of the spatial form contains the amino acid sequence of the (sub)domains IIa or IIb, III and/or IV of calpain.

7. The spatial form as claimed in any of the above-mentioned claims, wherein the spatial form is a crystal form, the crystal form comprising at least one polypeptide, containing at least one (sub)domain of a protein from the family consisting of the neutral Ca-activated cysteine proteinases (calpains), per asymmetric unit, which (sub)domain participates in the catalysis.

8. A crystal form of at least one polypeptide per asymmetric unit as claimed in claim 7, wherein the crystal form contains metal ions.

9. The crystal form of at least one polypeptide per asymmetric unit as claimed in either of claims 7 and 8, wherein the crystal form contains Ca ions and/or heavy metal ions.

10. The crystal form of at least one polypeptide per asymmetric unit as claimed in any of the above-mentioned claims 7 to 9, wherein the metal ions are situated in the spatial vicinity of cysteine or histidine residues of at least one polypeptide.

11. The crystal form of at least one polypeptide per asymmetric unit as claimed in any of the above-mentioned claims 7 to 10, wherein the crystal form contains at least one compound selected from the group consisting of substrate, pseudosubstrate, activator and inhibitor molecules.

12. The crystal form of at least one polypeptide per asymmetric unit as claimed in claim 11, wherein the compound is a di- or oligopeptide.

13. The crystal form of at least one polypeptide per asymmetric unit as claimed in claim 11 or 12, wherein the compound is a chemically modified di- or oligopeptide.

14. The crystal form of at least one polypeptide per asymmetric unit as claimed in any of the above-mentioned claims 7 to 13, wherein the crystal form comprises two different polypeptides as a heterodimer in the asymmetric unit.

15. The crystal form of at least one polypeptide per asymmetric unit as claimed in any of the above-mentioned claims 7 to 14, wherein at least one polypeptide contains an amino acid sequence as shown in FIG. 3, 4, 5 or 6.

16. The crystal form of at least one polypeptide per asymmetric unit as claimed in any of the above-mentioned claims 7 to 15, wherein the asymmetric unit has a heterodimer which contains a polypeptide (1) having an amino acid sequence as shown in FIG. 3 and a polypeptide (2) having an amino acid sequence as shown in FIG. 4.

17. The crystal form of at least one polypeptide per asymmetric unit as claimed in any of the above-mentioned claims 7 to 16, wherein the space group of the crystal having the crystal form is monoclinic, tetragonal, orthorhombic, cubic, triclinic, hexagonal or trigonal/rhombohedral.

18. The crystal form of at least one polypeptide per asymmetric unit as claimed in any of the above-mentioned claims 7 to 17, wherein the space group of the crystal having the crystal form is P2₁.

19. The crystal form of at least one polypeptide per asymmetric unit as claimed in any of the above-mentioned claims 7 to 18, wherein the unit cell of the crystal containing the crystal form has cell constants of about a 64.9 Å, b=134.0 Å, c=78.0 Å and β=102.4° or a=51.8 Å, b=171.4 Å, c=64.7 Å and β=94.80°.

20. The crystal form of at least one polypeptide per asymmetric unit as claimed in any of the above-mentioned claims 7 to 19, wherein the calpain subdomain IIa (sequence segment T93 to G209) and/or IIb (sequence segment G210 to N342) of the at least one polypeptide per asymmetric unit has the structural coordinates according to FIG. 10 for the above-mentioned amino acids, which subdomain participates in catalysis.

21. The crystal form of at least one polypeptide per asymmetric unit as claimed in any of the above-mentioned claims 7 to 20, wherein at least one polypeptide per asymmetric unit has the structural coordinates according to FIG. 10 for the large subunit (A2 to L700).

22. The crystal form of at least one polypeptide per asymmetric unit as claimed in any of the above-mentioned claims 7 to 21, wherein at least one polypeptide per asymmetric unit has the structural coordinates according to FIG. 10 for the large subunit (A2 to L700) and at least one other polypeptide has the structural coordinates according to FIG. 10 for the small subunit (T85 to S268).

23. The crystal form of at least one polypeptide per asymmetric unit as claimed in any of the above-mentioned claims 7 to 22, wherein the crystal having the crystal form is shown by X-ray structure analysis to have reflections up to a Bragg index of at least d=3.0 Å.

24. A compound having the property of acting as a substrate, pseudosubstrate, activator or inhibitor of a neutral Ca-activated cysteine proteinase (calpain), wherein the compound interacts with the main and/or side chains of amino acids of the catalytic domain or of amino acids of a segment of at least one polypeptide of the crystal form, which segment is relevant for regulating the active center.

25. The compound as claimed in claim 24, wherein the compound interacts with the structure of the main and/or side chains of the catalytic domain or of a segment of at least one polypeptide in a spatial or crystal form as obtained according to any of claims 1 to 23, which segment is relevant for regulating the active center.

26. The compound as claimed in claim 24 or 25, wherein the compound interacts with at least one amino acid of the sequence segment β2tβ3, of the acidic loop, of at least one polypeptide in a spatial or crystal form as obtained according to any of claims 1 to 23.

27. The compound as claimed in claim 26, wherein the compound has at least one positive charge and/or at least one positive partial charge and essentially prevents an interaction between the α₇II-helix and the sequence segment β2tβ3.

28. The compound as claimed in claim 26 or 27, wherein the compound is an activator of the catalytic activity of a calpain.

30. The compound as claimed in claim 24 or 25, wherein the compound essentially increases the interaction between the segment α₇II-helix and the sequence segment β2tβ3 of the at least one polypeptide in a spatial or crystal form as obtained according to any of claims 1 to 23.

31. The compound as claimed in claim 30, wherein the compound interacts with at least one of the amino acids 226L, 230L, 234L, 354L, 355L and/or 357L.

32. The compound as claimed in claim 30 or 31, wherein the compound is an inhibitor of the catalytic activity of the polypeptide.

33. The compound as claimed in claim 24 or 25, wherein the compound interacts with at least one of the amino acids of the subdomain(s) IIa and/or IIb.

34. The compound as claimed in claim 33, wherein the inhibitor blocks the rotational and/or translational movement of subdomain IIb relative to the subdomain IIa by becoming intercalated in the cleft between the two subdomains.

35. A method for identifying a compound having the property of acting as a substrate, pseudosubstrate, activator or inhibitor of a neutral Ca-activated cysteine proteinase (calpain), wherein
(a) a spatial or crystal form is obtained as claimed in any of claims 1 to 23,
(b) the structural coordinates of the spatial or crystal form are represented in three dimensions,
(c) steric properties and/or functional groups of a compound are chosen so that interactions between the compound and the main and/or side chains of the polypeptide are generated in the binding region and
(d) the compound obtained according to (c) is inserted into the active center of the catalytic subdomain(s) or into a polypeptide segment relevant for regulating the active center.

36. The method as claimed in claim 35, wherein the three-dimensional structure of the compound is determined in a method step (c1).

37. The method as claimed in claim 35 or 36, wherein the intensity of the interaction between the compound and at least one polypeptide, as obtained according to a spatial or crystal form as claimed in any of claims 1 to 23, is determined in a method step (d1).

38. The method as claimed in any of claims 35 to 37, wherein some or all of the structural coordinates from FIG. 10 are represented according to method step (b).

39. The method as claimed in any of claims 35 to 38, wherein the method steps (c), (c1), (d) and (d1) are repeated cyclically until the intensity, obtained according to (d1), of the interaction between compound and the main and/or side chain of the at least one polypeptide in a spatial or crystal form as obtained according to any of claims 1 to 23 is optimized.

40. The method as claimed in any of claims 35 to 39, wherein the properties of the compound are determined in a biological test system in a method step (d2).

41. A method for identifying a compound having the property of acting as a substrate, pseudosubstrate, activator or inhibitor of a neutral Ca-activated cysteine proteinase (calpain), wherein
(a) a biological test system for a substrate, pseudosubstrate, activator and/or inhibitor of calpain is established,
(b) a compound acting as a substrate, pseudosubstrate, activator and/or inhibitor of calpain is determined by a biological test system according to (a),
(c) the conformation of the compound is determined,
(d) the structural coordinates of at least one polypeptide from a spatial or crystal form as claimed in any of claims 1 to 23 are represented and
(e) the structure of the compound, obtained according to (b) and (c) is inserted into the structure, obtained according to (d), of the active center of the catalytic subdomain(s) or of a polypeptide segment relevant for regulating the active center.

42. The method as claimed in claim 41, wherein the type and/or intensity of the interaction between the compound and the spatial or crystal form of at least one polypeptide are determined in a method step (e1).

43. A compound as a substrate, pseudosubstrate, activator or inhibitor, wherein said compound is obtained from a method as claimed in any of claims 35 to 42.

44. A process for the preparation of a crystal form of at least one polypeptide as claimed in any of claims 1 to 23, wherein
(a) the polypeptide is overexpressed in an expression system,
(b) the polypeptide obtained according to (a) is dissolved in a suitable buffer system and
(c) the crystallization is initiated by, for example, vapor diffusion methods.

45. A method for representing a three-dimensional structure of a polypeptide or of a complex of unknown structure, containing at least one polypeptide which contains at least one domain of a protein from the family consisting of the neutral Ca-activated cysteine proteinases (calpains), which domain participates in the catalysis, wherein the unknown structure of the polypeptide or complex is determined on the basis of a known spatial or crystal form as claimed in any of claims 1 to 23.

46. The method as claimed in claim 45, wherein the structural coordinates as shown in FIG. 10 are used.

47. The method as claimed in claim 45 or 46, wherein
(a) the primary sequence of a polypeptide of unknown 3D structure is compared with the primary sequence of a polypeptide of known crystal form,
(b) the 3D structure of the polypeptide of unknown structure is modeled on the basis of the crystal form of homologous segments and
(c) energy optimizations of the structure modeled according to (b) are carried out with the aid of appropriate computer programs.

48. The method as claimed in any of claims 45 to 47, wherein the polypeptide of unknown structure is an isozyme from the family consisting of the n-calpains or an isozyme of an m- or μ-calpain.

49. A method for identifying a substrate, pseudosubstrate, activator or inhibitor of a neutral Ca-activated cysteine proteinase (calpain) of unknown 3D structure, wherein
(a) the unknown 3D structure of the polypeptide is determined by a method as claimed in any of claims 45 to 48 and
(b) a compound having the property of acting as an inhibitor, pseudosubstrate, activator or substrate of the polypeptide of unknown 3D structure is determined with the aid of a method as claimed in any of claims 35 to 42.

50. The use of inhibitors and/or activators of the catalytic activity of a neutral Ca-activated cysteine proteinase (calpain) as claimed in any of claims 24 to 34 or claim 43, or obtained from a method as claimed in claim 49, as drugs.

51. The use of compounds as claimed in claim 50 for the treatment of ischemic conditions, muscular dystrophy and/or tumor diseases.
